SEQUENCE LISTING (833 sequences)

SEQ ID NO: 1 - protein, 118 aa, Artificial Sequence
  source: /note=“Description of Artificial Sequence: Synthetic polypeptide”
QVQLQQPGAELVKPGASVKMSCKASGYTFTSYNMHWIKQTPGQGLEWIGDIYPGNGDTSYNQKFKGKATLTADKSSSTVYMQLSSLTSEDSAVYYCARVGGAFPMDYWGQGTSVTVSS

SEQ ID NO: 2 - protein, 111 aa, Artificial Sequence
  source: /note=“Description of Artificial Sequence: Synthetic polypeptide”
DIVLTQSPASLAVSLGQRATISCRASESVEYYGTSLMQWYQQKPGQPPKLLIYAASNVESGVPARFSGSGSGTDFSLNIHPVEEDDIAIYFCQQSRKDPSTFGGGTKLEIK

SEQ ID NO: 3 - protein, 5 aa, Artificial Sequence
  source: /note=“Description of Artificial Sequence: Synthetic peptide”
SYNMH

SEQ ID NO: 4 - protein, 17 aa, Artificial Sequence
  source: /note=“Description of Artificial Sequence: Synthetic peptide”
DIYPGNGDTSYNQKFKG

SEQ ID NO: 5 - protein, 9 aa, Artificial Sequence
  source: /note=“Description of Artificial Sequence: Synthetic peptide”
VGGAFPMDY

SEQ ID NO: 6 - protein, 15 aa, Artificial Sequence
  source: /note=“Description of Artificial Sequence: Synthetic peptide”
RASESVEYYGTSLMQ

SEQ ID NO: 7 - protein, 7 aa, Artificial Sequence
  source: /note=“Description of Artificial Sequence: Synthetic peptide”
AASNVES

SEQ ID NO: 8 - protein, 9 aa, Artificial Sequence
  source: /note=“Description of Artificial Sequence: Synthetic peptide”
QQSRKDPST

SEQ ID NO: 9 - protein, 7 aa, Artificial Sequence
  source: /note=“Description of Artificial Sequence: Synthetic peptide”
GYTFTSY

SEQ ID NO: 10 - protein, 6 aa, Artificial Sequence
  source: /note=“Description of Artificial Sequence: Synthetic peptide”
YPGNGD

SEQ ID NO: 11 - DNA, 354 bp, Artificial Sequence
  source: /note=“Description of Artificial Sequence: Synthetic polynucleotide”
caggtgcaactgcagcagcctggggctgagctggtgaagcctggggcctcagtgaagatgtcctgcaaggcttctggctacacatttaccagttacaatatgcactggataaagcagacacctggacagggcctggaatggattggagatatttatccaggaaatggtgatacttcctacaatcagaaattcaaaggcaaggccacattgactgcagacaaatcctccagcacagtctacatgcagctcagcagcctgacatctgaggactctgcggtctattactgtgcaagagtggggggtgcctttcctatggactactggggtcaaggaacctcagtcaccgtctcctca

SEQ ID NO: 12 - protein, 11 aa, Artificial Sequence
  source: /note=“Description of Artificial Sequence: Synthetic peptide”
SESVEYYGTSL

SEQ ID NO: 13 - protein, 3 aa, Artificial Sequence
  source: /note=“Description of Artificial Sequence: Synthetic peptide”
AAS

SEQ ID NO: 14 - protein, 6 aa, Artificial Sequence
  source: /note=“Description of Artificial Sequence: Synthetic peptide”
SRKDPS

SEQ ID NO: 15 - DNA, 333 bp, Artificial Sequence
  source: /note=“Description of Artificial Sequence: Synthetic polynucleotide”
gacattgtgctcacccaatctccagcttctttggctgtgtctctagggcagagagccaccatctcctgcagagccagtgaaagtgttgaatattatggcacaagtttaatgcagtggtaccaacagaaaccaggacagccacccaaactcctcatctatgctgcatccaacgtagaatctggggtccctgccaggtttagtggcagtgggtctgggacagacttcagcctcaacatccatcctgtggaggaggatgatattgcaatatatttctgtcagcaaagtaggaaggatccttcgacgttcggtggaggcaccaagctggagatcaaa

SEQ ID NO: 16 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGNGDTSYNQKFKGRATMTADKSTSTVYMELSSLRSEDTAVYYCARVGGAFPMDYWGQGTLVTVSS

SEQ ID NO: 17 - DNA, 354 bp, Artificial Sequence
  source: /note=“Description of Artificial Sequence: Synthetic polynucleotide”
caggtgcagctggtgcagtcaggcgccgaagtgaagaaacccggcgctagtgtgaaagtctcttgtaaagctagtggctacaccttcactagctataatatgcactgggttcgccaggccccagggcagggcctcgagtggatcggcgatatctaccccgggaacggcgacactagttataatcagaagtttaagggtagagctactatgaccgccgataagtctactagcaccgtctatatggaactgagttccctgaggtctgaggacaccgccgtctactactgcgctagagtgggcggagccttccctatggactactggggtcaaggcaccctggtcaccgtgtctagc

SEQ ID NO: 18 - protein, 444 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGNGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - DNA, 1332 bp, Artificial Sequence
  source: /note=“Description of Artificial Sequence: Synthetic polynucleotide”
caggtgcagctggtgcagtcaggcgccgaagtgaagaaacccggcgctagtgtgaaagtctcttgtaaagctagtggctacaccttcactagctataatatgcactgggttcgccaggccccagggcagggcctcgagtggatcggcgatatctaccccgggaacggcgacactagttataatcagaagtttaagggtagagctactatgaccgccgataagtctactagcaccgtctatatggaactgagttccctgaggtctgaggacaccgccgtctactactgcgctagagtgggcggagccttccctatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 20 - protein, 111 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIK

SEQ ID NO: 21 - DNA, 333 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaag

SEQ ID NO: 22 - protein, 218 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 654 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctactactgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 24 - protein, 17 aa, Artificial Sequence
  source: /note=“Description of Artificial Sequence: Synthetic peptide”
DIYPGSGDTSYNQKFKG

SEQ ID NO: 25 - protein, 6 aa, Artificial Sequence
  source: /note=“Description of Artificial Sequence: Synthetic peptide”
YPGSGD

SEQ ID NO: 26 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGSGDTSYNQKFKGRATMTADKSTSTVYMELSSLRSEDTAVYYCARVGGAFPMDYWGQGTLVTVSS

SEQ ID NO: 27 - DNA, 354 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctacaccttcactagctataatatgcactgggttcgccaggccccaggtcaaggcctcgagtggatcggcgatatctaccccggtagcggcgacactagttataatcagaagtttaagggtagagctactatgaccgccgataagtctactagcaccgtctatatggaactgagttccctgaggtctgaagataccgccgtctactactgcgctagagtgggcggagccttccctatggactactggggtcaaggcaccctggtcaccgtgtctagc

SEQ ID NO: 28 - protein, 444 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGSGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - DNA, 1332 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctacaccttcactagctataatatgcactgggttcgccaggccccaggtcaaggcctcgagtggatcggcgatatctaccccggtagcggcgacactagttataatcagaagtttaagggtagagctactatgaccgccgataagtctactagcaccgtctatatggaactgagttccctgaggtctgaagataccgccgtctactactgcgctagagtgggcggagccttccctatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 30 - protein, 17 aa, Artificial Sequence
  source: /note=“Description of Artificial Sequence: Synthetic peptide”
DIYPGQGDTSYNQKFKG

SEQ ID NO: 31 - protein, 6 aa, Artificial Sequence
  source: /note=“Description of Artificial Sequence: Synthetic peptide”
YPGQGD

SEQ ID NO: 32 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSS

SEQ ID NO: 33 - DNA, 354 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagc

SEQ ID NO: 34 - protein, 444 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 35 - DNA, 1332 bp, Artificial Sequence
  source: /note=“Description of Artificial Sequence: Synthetic polynucleotide”
caggtgcagctggtgcagtcaggcgccgaagtgaagaaacccggcgctagtgtgaaagttagctgtaaagctagtggctatactttcacttcttataatatgcactgggtccgccaggccccaggtcaaggcctcgagtggatcggcgatatctaccccggtcaaggcgacacttcctataatcagaagtttaagggtagagctactatgaccgccgataagtctacttctaccgtctatatggaactgagttccctgaggtctgaggacaccgccgtctactactgcgctagagtgggcggagccttcccaatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 36 - protein, 118 aa, Artificial Sequence
  source: /note=“Description of Artificial Sequence: Synthetic polypeptide”
QVQLVQSGAEVKKPGASVKVSCKASGYTFTSYNMHWIRQAPGQGLEWIGDIYPGNGDTSYNQKFKGRATLTADKSTSTVYMELSSLRSEDTAVYYCARVGGAFPMDYWGQGTLVTVSS

SEQ ID NO: 37 - DNA, 354 bp, Artificial Sequence
  source: /note=“Description of Artificial Sequence: Synthetic polynucleotide”
caggtgcagctggtgcagtcaggcgccgaagtgaagaaacccggcgctagtgtgaaagtttcttgtaaagctagtggctacaccttcactagctataatatgcactggattagacaggccccagggcagggcctcgagtggatcggcgatatctaccccgggaacggcgacactagttataatcagaagtttaagggtagagctaccctgaccgccgataagtctactagcaccgtctatatggaactgagttccctgaggtctgaggacaccgccgtctactactgcgctagagtgggcggagccttccctatggactactgggggcagggcaccctggtcaccgtgtctagc

SEQ ID NO: 38 - protein, 444 aa, Artificial Sequence
  source: /note=“Description of Artificial Sequence: Synthetic polypeptide”
QVQLVQSGAEVKKPGASVKVSCKASGYTFTSYNMHWIRQAPGQGLEWIGDIYPGNGDTSYNQKFKGRATLTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 39 - DNA, 1332 bp, Artificial Sequence
  source: /note=“Description of Artificial Sequence: Synthetic polynucleotide”
caggtgcagctggtgcagtcaggcgccgaagtgaagaaacccggcgctagtgtgaaagtttcttgtaaagctagtggctacaccttcactagctataatatgcactggattagacaggccccagggcagggcctcgagtggatcggcgatatctaccccgggaacggcgacactagttataatcagaagtttaagggtagagctaccctgaccgccgataagtctactagcaccgtctatatggaactgagttccctgaggtctgaggacaccgccgtctactactgcgctagagtgggcggagccttccctatggactactgggggcagggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 40 - protein, 111 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYFCQQSRKDPSTFGGGTKVEIK

SEQ ID NO: 41 - DNA, 333 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctacttctgtcagcagtctaggaaggaccctagcaccttcggcggaggcactaaggtcgagattaag

SEQ ID NO: 42 - protein, 218 aa, Artificial Sequence
  source: /note=“Description of Artificial Sequence: Synthetic polypeptide”
DIVLTQSPDSLAVSLGERATINCRASESVEYYGTSLMQWYQQKPGQPPKLLIYAASNVESGVPDRFSGSGSGTDFTLTISSLQAEDVAVYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - DNA, 654 bp, Artificial Sequence
  source: /note=“Description of Artificial Sequence: Synthetic polynucleotide”
gatatcgtcctgactcagtcacccgatagcctggccgtcagcctgggcgagcgggctactattaactgtagagctagtgaatcagtcgagtactacggcactagcctgatgcagtggtatcagcagaagcccggtcaaccccctaagctgctgatctacgccgcctctaacgtggaatcaggcgtgcccgataggtttagcggtagcggtagtggcaccgacttcaccctgactattagtagcctgcaggccgaggacgtggccgtctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 44 - protein, 118 aa, Artificial Sequence
  source: /note=“Description of Artificial Sequence: Synthetic polypeptide”
QVQLVQSGAEVKKPGASVKVSCKASGYTFTSYNMHWIRQAPGQGLEWIGDIYPGSGDTSYNQKFKGRATLTADKSTSTVYMELSSLRSEDTAVYYCARVGGAFPMDYWGQGTLVTVSS

SEQ ID NO: 45 - DNA, 354 bp, Artificial Sequence
  source: /note=“Description of Artificial Sequence: Synthetic polynucleotide”
caggtgcagctggtgcagtcaggcgccgaagtgaagaaacccggcgcaagcgttaaagtctcatgtaaagctagtggctacaccttcactagctataatatgcactggattagacaggccccagggcaaggcctggagtggatcggcgatatctaccccggtagcggcgacactagttataatcagaagtttaagggtagagctaccctgaccgccgataagtctactagcaccgtctatatggaactgagttccctgaggagtgaagacaccgccgtctactactgcgctagagtgggcggagccttccctatggactactggggtcaaggcaccctggtcaccgtgtcaagc

SEQ ID NO: 46 - protein, 444 aa, Artificial Sequence
  source: /note=“Description of Artificial Sequence: Synthetic polypeptide”
QVQLVQSGAEVKKPGASVKVSCKASGYTFTSYNMHWIRQAPGQGLEWIGDIYPGSGDTSYNQKFKGRATLTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 47 - DNA, 1332 bp, Artificial Sequence
  source: /note=“Description of Artificial Sequence: Synthetic polynucleotide”
caggtgcagctggtgcagtcaggcgccgaagtgaagaaacccggcgcaagcgttaaagtctcatgtaaagctagtggctacaccttcactagctataatatgcactggattagacaggccccagggcaaggcctggagtggatcggcgatatctaccccggtagcggcgacactagttataatcagaagtttaagggtagagctaccctgaccgccgataagtctactagcaccgtctatatggaactgagttccctgaggagtgaagacaccgccgtctactactgcgctagagtgggcggagccttccctatggactactggggtcaaggcaccctggtcaccgtgtcaagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 48 - protein, 118 aa, Artificial Sequence
  source: /note=“Description of Artificial Sequence: Synthetic polypeptide”
QVQLVQSGAEVKKPGASVKVSCKASGYTFTSYNMHWIRQAPGQGLEWIGDIYPGQGDTSYNQKFKGRATLTADKSTSTVYMELSSLRSEDTAVYYCARVGGAFPMDYWGQGTLVTVSS

SEQ ID NO: 49 - DNA, 354 bp, Artificial Sequence
  source: /note=“Description of Artificial Sequence: Synthetic polynucleotide”
caggtgcagctggtgcagtcaggcgccgaagtgaagaaacccggcgctagtgtgaaagtctcttgtaaagctagtggctacaccttcactagctataatatgcactggattagacaggccccaggtcaaggcctcgagtggatcggcgatatctaccccggtcaaggcgacactagttataatcagaagtttaagggtagagctaccctgaccgccgataagtctactagcaccgtctatatggaactgagttccctgaggtctgaggacaccgccgtctactactgcgctagagtgggcggagccttccctatggactactggggtcaaggcaccctggtcaccgtgtctagc

SEQ ID NO: 50 - protein, 444 aa, Artificial Sequence
  source: /note=“Description of Artificial Sequence: Synthetic polypeptide”
QVQLVQSGAEVKKPGASVKVSCKASGYTFTSYNMHWIRQAPGQGLEWIGDIYPGQGDTSYNQKFKGRATLTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 51 - DNA, 1332 bp, Artificial Sequence
  source: /note=“Description of Artificial Sequence: Synthetic polynucleotide”
caggtgcagctggtgcagtcaggcgccgaagtgaagaaacccggcgctagtgtgaaagtctcttgtaaagctagtggctacaccttcactagctataatatgcactggattagacaggccccaggtcaaggcctcgagtggatcggcgatatctaccccggtcaaggcgacactagttataatcagaagtttaagggtagagctaccctgaccgccgataagtctactagcaccgtctatatggaactgagttccctgaggtctgaggacaccgccgtctactactgcgctagagtgggcggagccttccctatggactactggggtcaaggcaccctggtcaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 52 - protein, 118 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSS

SEQ ID NO: 53 - DNA, 354 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagc

SEQ ID NO: 54 - protein, 444 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGNGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 55 - DNA, 1332 bp, Artificial Sequence
  source: /note=“Description of Artificial Sequence: Synthetic polynucleotide”
caggtgcagctggtgcagtcaggcgccgaagtgaagaaacccggctctagcgtgaaagtttcttgtaaagctagtggctacaccttcactagctataatatgcactgggttcgccaggccccagggcaaggcctcgagtggatgggcgatatctaccccgggaacggcgacactagttataatcagaagtttaagggtagagtcactatcaccgccgataagtctactagcaccgtctatatggaactgagttccctgaggtctgaggacaccgccgtctactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 56 - protein, 111 aa, Artificial Sequence
  source: /note=“Description of Artificial Sequence: Synthetic polypeptide”
EIVLTQSPATLSLSPGERATLSCRASESVEYYGTSLMQWYQQKPGQAPRLLIYAASNVESGIPARFSGSGSGTDFTLTISSLEPEDIAVYFCQQSRKDPSTFGGGTKVEIK

SEQ ID NO: 57 - DNA, 333 bp, Artificial Sequence
  source: /note=“Description of Artificial Sequence: Synthetic polynucleotide”
gagatcgtcctgactcagtcacccgctaccctgagcctgagccctggcgagagagctacactgagctgtagagctagtgaatcagtcgagtactacggcactagcctgatgcagtggtatcagcagaagcccggtcaagcccctagactgctgatctacgccgcctctaacgtggaatcagggatccccgctaggtttagcggtagcggtagtggcaccgacttcaccctgactatctctagcctggaacccgaggatatcgccgtctacttctgtcagcagtctaggaaggaccctagcaccttcggcggaggcactaaggtcgagattaag

SEQ ID NO: 58 - protein, 218 aa, Artificial Sequence
  source: /note=“Description of Artificial Sequence: Synthetic polypeptide”
EIVLTQSPATLSLSPGERATLSCRASESVEYYGTSLMQWYQQKPGQAPRLLIYAASNVESGIPARFSGSGSGTDFTLTISSLEPEDIAVYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 654 bp, Artificial Sequence
  source: /note=“Description of Artificial Sequence: Synthetic polynucleotide”
gagatcgtcctgactcagtcacccgctaccctgagcctgagccctggcgagagagctacactgagctgtagagctagtgaatcagtcgagtactacggcactagcctgatgcagtggtatcagcagaagcccggtcaagcccctagactgctgatctacgccgcctctaacgtggaatcagggatccccgctaggtttagcggtagcggtagtggcaccgacttcaccctgactatctctagcctggaacccgaggatatcgccgtctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 60 - protein, 118 aa, Artificial Sequence
  source: /note=“Description of Artificial Sequence: Synthetic polypeptide”
EVQLVQSGAEVKKPGESLKISCKGSGYTFTSYNMHWVRQMPGKGLEWMGDIYPGNGDTSYNQKFKGQVTISADKSISTVYLQWSSLKASDTAMYYCARVGGAFPMDYWGQGTTVTVSS

SEQ ID NO: 61 - DNA, 354 bp, Artificial Sequence
  source: /note=“Description of Artificial Sequence: Synthetic polynucleotide”
gaagtgcagctggtgcagtcaggcgccgaagtgaagaagcccggcgagtcactgaagattagctgtaaaggttcaggctacaccttcactagctataatatgcactgggtccgccagatgcccgggaaaggcctcgagtggatgggcgatatctaccccgggaacggcgacactagttataatcagaagtttaaggggcaagtcacaattagcgccgataagtctattagcaccgtctacctgcagtggtctagcctgaaggctagtgacaccgctatgtactactgcgctagagtgggcggagccttccctatggactactggggtcaaggcactaccgtgaccgtgtctagc

SEQ ID NO: 62 - protein, 444 aa, Artificial Sequence
  source: /note=“Description of Artificial Sequence: Synthetic polypeptide”
EVQLVQSGAEVKKPGESLKISCKGSGYTFTSYNMHWVRQMPGKGLEWMGDIYPGNGDTSYNQKFKGQVTISADKSISTVYLQWSSLKASDTAMYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 63 - DNA, 1332 bp, Artificial Sequence
  source: /note=“Description of Artificial Sequence: Synthetic polynucleotide”
gaagtgcagctggtgcagtcaggcgccgaagtgaagaagcccggcgagtcactgaagattagctgtaaaggttcaggctacaccttcactagctataatatgcactgggtccgccagatgcccgggaaaggcctcgagtggatgggcgatatctaccccgggaacggcgacactagttataatcagaagtttaaggggcaagtcacaattagcgccgataagtctattagcaccgtctacctgcagtggtctagcctgaaggctagtgacaccgctatgtactactgcgctagagtgggcggagccttccctatggactactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 64 - protein, 111 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIK

SEQ ID NO: 65 - DNA, 333 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaag

SEQ ID NO: 66 - protein, 218 aa, Artificial Sequence
  source: /note=“Description of Artificial Sequence: Synthetic polypeptide”
AIQLTQSPSSLSASVGDRVTITCRASESVEYYGTSLMQWYQQKPGKAPKLLIYAASNVESGVPSRFSGSGSGTDFTLTISSLQPEDFATYFCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 654 bp, Artificial Sequence
  source: /note=“Description of Artificial Sequence: Synthetic polynucleotide”
gctattcagctgactcagtcacctagtagcctgagcgctagtgtgggcgatagagtgactatcacctgtagagctagtgaatcagtcgagtactacggcactagcctgatgcagtggtatcagcagaagcccgggaaagcccctaagctgctgatctacgccgcctctaacgtggaatcaggcgtgccctctaggtttagcggtagcggtagtggcaccgacttcaccctgactatctctagcctgcagcccgaggacttcgctacctacttctgtcagcagtctaggaaggaccctagcaccttcggcggaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 68 - protein, 118 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGSGDTSYNQKFKGRVTITADKSTSTVYMELSSLRSEDTAVYYCARVGGAFPMDYWGQGTTVTVSS

SEQ ID NO: 69 - DNA, 354 bp, Artificial Sequence
  source: /note=“Description of Artificial Sequence: Synthetic polynucleotide”
caggtgcaattggttcagtcaggagcagaagttaagaagccaggatcatccgtcaaggtgtcctgcaaagcatctggctacaccttcaccagctacaatatgcactgggtccgacaagcccctgggcagggcttggagtggatgggagacatttaccccggcagtggtgacacttcctataaccagaagttcaagggccgagtcactattaccgctgacaagtccacctccacagtctacatggaactctcttctctgagatccgaggacactgccgtctattactgcgctcgcgtgggcggtgctttcccaatggactattggggacagggcacaaccgtgaccgtcagctca

SEQ ID NO: 70 - protein, 445 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGSGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - DNA, 1335 bp, Artificial Sequence
  source: /note=“Description of Artificial Sequence: Synthetic polynucleotide”
caggtgcaattggttcagtcaggagcagaagttaagaagccaggatcatccgtcaaggtgtcctgcaaagcatctggctacaccttcaccagctacaatatgcactgggtccgacaagcccctgggcagggcttggagtggatgggagacatttaccccggcagtggtgacacttcctataaccagaagttcaagggccgagtcactattaccgctgacaagtccacctccacagtctacatggaactctcttctctgagatccgaggacactgccgtctattactgcgctcgcgtgggcggtgctttcccaatggactattggggacagggcacaaccgtgaccgtcagctcagcctctacaaagggcccctccgtctttccactcgcgccgtgctctcgctccacctcagagtcaactgccgctctgggttgcctggtcaaggactacttcccagagccggtgacagtgagctggaacagtggggccctgacatccggcgttcataccttccccgcagtcctccagtcctcaggcctgtattccctgagcagcgttgtcacagtgccctccagctctcttggcacgaaaacctacacatgcaacgttgatcataagccgtctaataccaaggtggataaaagagtggagagcaagtacggcccaccctgcccgccttgcccagctccggagttcctgggcggaccatccgttttcttgtttccacccaaacctaaagacactctgatgatttcccgaacccctgaagtgacttgcgttgtggtggacgtctcccaggaggacccagaagtgcaattcaactggtacgtggacggggtggaggtgcacaatgcaaaaaccaaaccaagggaggaacagtttaattcaacatatagggttgtgtctgtgctgacggttctgcatcaggactggctgaacggaaaggaatacaagtgcaaggtgtccaacaaaggactgccaagctctatcgagaaaacaatctctaaggccaagggacaacctagagagccccaagtttacaccctgccaccatcacaggaagagatgaccaaaaatcaggtgagcttgacatgcctggtgaagggcttctaccctagcgatattgcggttgagtgggagtcaaatggccagcctgagaacaactataagactactcctcccgtgctggactccgacgggagctttttcctgtattccaggcttacagtcgataagagcagatggcaagaggggaatgtgttttcctgctccgtgatgcacgaggctctccataaccattatactcagaaaagtctctctctgtcactgggcaaa

SEQ ID NO: 72 - protein, 118 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGQGDTSYNQKFKGRVTITADKSTSTVYMELSSLRSEDTAVYYCARVGGAFPMDYWGQGTTVTVSS

SEQ ID NO: 73 - DNA, 354 bp, Artificial Sequence
  source: /note=“Description of Artificial Sequence: Synthetic polynucleotide”
caggtgcaattggttcagtcaggagcagaagttaagaagccaggatcatccgtcaaggtgtcctgcaaagcatctggctacaccttcaccagctacaatatgcactgggtccgacaagcccctgggcagggcttggagtggatgggagacatttaccccggccagggtgacacttcctataaccagaagttcaagggccgagtcactattaccgctgacaagtccacctccacagtctacatggaactctcttctctgagatccgaggacactgccgtctattactgcgctcgcgtgggcggtgctttcccaatggactattggggacagggcacaaccgtgaccgtcagctca

SEQ ID NO: 74 - protein, 445 aa, Artificial Sequence
  source: /note=“Description of Artificial Sequence: Synthetic polypeptide”
QVQLVQSGAEVKKPGSSVKVSCKASGYTFTSYNMHWVRQAPGQGLEWMGDIYPGQGDTSYNQKFKGRVTITADKSTSTVYMELSSLRSEDTAVYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - DNA, 1335 bp, Artificial Sequence
  source: /note=“Description of Artificial Sequence: Synthetic polynucleotide”
caggtgcaattggttcagtcaggagcagaagttaagaagccaggatcatccgtcaaggtgtcctgcaaagcatctggctacaccttcaccagctacaatatgcactgggtccgacaagcccctgggcagggcttggagtggatgggagacatttaccccggccagggtgacacttcctataaccagaagttcaagggccgagtcactattaccgctgacaagtccacctccacagtctacatggaactctcttctctgagatccgaggacactgccgtctattactgcgctcgcgtgggcggtgctttcccaatggactattggggacagggcacaaccgtgaccgtcagctcagcctctacaaagggcccctccgtctttccactcgcgccgtgctctcgctccacctcagagtcaactgccgctctgggttgcctggtcaaggactacttcccagagccggtgacagtgagctggaacagtggggccctgacatccggcgttcataccttccccgcagtcctccagtcctcaggcctgtattccctgagcagcgttgtcacagtgccctccagctctcttggcacgaaaacctacacatgcaacgttgatcataagccgtctaataccaaggtggataaaagagtggagagcaagtacggcccaccctgcccgccttgcccagctccggagttcctgggcggaccatccgttttcttgtttccacccaaacctaaagacactctgatgatttcccgaacccctgaagtgacttgcgttgtggtggacgtctcccaggaggacccagaagtgcaattcaactggtacgtggacggggtggaggtgcacaatgcaaaaaccaaaccaagggaggaacagtttaattcaacatatagggttgtgtctgtgctgacggttctgcatcaggactggctgaacggaaaggaatacaagtgcaaggtgtccaacaaaggactgccaagctctatcgagaaaacaatctctaaggccaagggacaacctagagagccccaagtttacaccctgccaccatcacaggaagagatgaccaaaaatcaggtgagcttgacatgcctggtgaagggcttctaccctagcgatattgcggttgagtgggagtcaaatggccagcctgagaacaactataagactactcctcccgtgctggactccgacgggagctttttcctgtattccaggcttacagtcgataagagcagatggcaagaggggaatgtgttttcctgctccgtgatgcacgaggctctccataaccattatactcagaaaagtctctctctgtcactgggcaaa

SEQ ID NO: 76 - protein, 118 aa, Artificial Sequence
  source: /note=“Description of Artificial Sequence: Synthetic polypeptide”
EVQLVQSGAEVKKPGESLKISCKGSGYTFTSYNMHWVRQMPGKGLEWMGDIYPGSGDTSYNQKFKGQVTISADKSISTVYLQWSSLKASDTAMYYCARVGGAFPMDYWGQGTTVTVSS

SEQ ID NO: 77 - DNA, 354 bp, Artificial Sequence
  source: /note=“Description of Artificial Sequence: Synthetic polynucleotide”
gaagttcaattggtacagtctggcgcagaagtaaagaaaccaggagagagtttgaaaatttcctgcaagggcagtgggtacacattcacgtcctacaatatgcactgggtgagacagatgccaggcaagggcctggagtggatgggagacatatacccaggcagtggagacacaagctataatcagaaattcaaaggacaggtgacgatctccgcagacaaatccatatctacggtctacctccagtggtcctcacttaaagcctccgacaccgccatgtactattgcgctcgggtaggtggcgcgtttccaatggactattggggccaagggaccacagtaaccgtcagctca

SEQ ID NO: 78 - protein, 445 aa, Artificial Sequence
  source: /note=“Description of Artificial Sequence: Synthetic polypeptide”
EVQLVQSGAEVKKPGESLKISCKGSGYTFTSYNMHWVRQMPGKGLEWMGDIYPGSGDTSYNQKFKGQVTISADKSISTVYLQWSSLKASDTAMYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - DNA, 1335 bp, Artificial Sequence
  source: /note=“Description of Artificial Sequence: Synthetic polynucleotide”
gaagttcaattggtacagtctggcgcagaagtaaagaaaccaggagagagtttgaaaatttcctgcaagggcagtgggtacacattcacgtcctacaatatgcactgggtgagacagatgccaggcaagggcctggagtggatgggagacatatacccaggcagtggagacacaagctataatcagaaattcaaaggacaggtgacgatctccgcagacaaatccatatctacggtctacctccagtggtcctcacttaaagcctccgacaccgccatgtactattgcgctcgggtaggtggcgcgtttccaatggactattggggccaagggaccacagtaaccgtcagctcagcctctacaaagggcccctccgtctttccactcgcgccgtgctctcgctccacctcagagtcaactgccgctctgggttgcctggtcaaggactacttcccagagccggtgacagtgagctggaacagtggggccctgacatccggcgttcataccttccccgcagtcctccagtcctcaggcctgtattccctgagcagcgttgtcacagtgccctccagctctcttggcacgaaaacctacacatgcaacgttgatcataagccgtctaataccaaggtggataaaagagtggagagcaagtacggcccaccctgcccgccttgcccagctccggagttcctgggcggaccatccgttttcttgtttccacccaaacctaaagacactctgatgatttcccgaacccctgaagtgacttgcgttgtggtggacgtctcccaggaggacccagaagtgcaattcaactggtacgtggacggggtggaggtgcacaatgcaaaaaccaaaccaagggaggaacagtttaattcaacatatagggttgtgtctgtgctgacggttctgcatcaggactggctgaacggaaaggaatacaagtgcaaggtgtccaacaaaggactgccaagctctatcgagaaaacaatctctaaggccaagggacaacctagagagccccaagtttacaccctgccaccatcacaggaagagatgaccaaaaatcaggtgagcttgacatgcctggtgaagggcttctaccctagcgatattgcggttgagtgggagtcaaatggccagcctgagaacaactataagactactcctcccgtgctggactccgacgggagctttttcctgtattccaggcttacagtcgataagagcagatggcaagaggggaatgtgttttcctgctccgtgatgcacgaggctctccataaccattatactcagaaaagtctctctctgtcactgggcaaa

SEQ ID NO: 80 - protein, 118 aa, Artificial Sequence
  source: /note=“Description of Artificial Sequence: Synthetic polypeptide”
EVQLVQSGAEVKKPGESLKISCKGSGYTFTSYNMHWVRQMPGKGLEWMGDIYPGQGDTSYNQKFKGQVTISADKSISTVYLQWSSLKASDTAMYYCARVGGAFPMDYWGQGTTVTVSS

SEQ ID NO: 81 - DNA, 354 bp, Artificial Sequence
  source: /note=“Description of Artificial Sequence: Synthetic polynucleotide”
gaagttcaattggtacagtctggcgcagaagtaaagaaaccaggagagagtttgaaaatttcctgcaagggcagtgggtacacattcacgtcctacaatatgcactgggtgagacagatgccaggcaagggcctggagtggatgggagacatatacccaggccagggagacacaagctataatcagaaattcaaaggacaggtgacgatctccgcagacaaatccatatctacggtctacctccagtggtcctcacttaaagcctccgacaccgccatgtactattgcgctcgggtaggtggcgcgtttccaatggactattggggccaagggaccacagtaaccgtcagctca

SEQ ID NO: 82 - protein, 445 aa, Artificial Sequence
  source: /note=“Description of Artificial Sequence: Synthetic polypeptide”
EVQLVQSGAEVKKPGESLKISCKGSGYTFTSYNMHWVRQMPGKGLEWMGDIYPGQGDTSYNQKFKGQVTISADKSISTVYLQWSSLKASDTAMYYCARVGGAFPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - DNA, 1335 bp, Artificial Sequence
  source: /note=“Description of Artificial Sequence: Synthetic polynucleotide”
gaagttcaattggtacagtctggcgcagaagtaaagaaaccaggagagagtttgaaaatttcctgcaagggcagtgggtacacattcacgtcctacaatatgcactgggtgagacagatgccaggcaagggcctggagtggatgggagacatatacccaggccagggagacacaagctataatcagaaattcaaaggacaggtgacgatctccgcagacaaatccatatctacggtctacctccagtggtcctcacttaaagcctccgacaccgccatgtactattgcgctcgggtaggtggcgcgtttccaatggactattggggccaagggaccacagtaaccgtcagctcagcctctacaaagggcccctccgtctttccactcgcgccgtgctctcgctccacctcagagtcaactgccgctctgggttgcctggtcaaggactacttcccagagccggtgacagtgagctggaacagtggggccctgacatccggcgttcataccttccccgcagtcctccagtcctcaggcctgtattccctgagcagcgttgtcacagtgccctccagctctcttggcacgaaaacctacacatgcaacgttgatcataagccgtctaataccaaggtggataaaagagtggagagcaagtacggcccaccctgcccgccttgcccagctccggagttcctgggcggaccatccgttttcttgtttccacccaaacctaaagacactctgatgatttcccgaacccctgaagtgacttgcgttgtggtggacgtctcccaggaggacccagaagtgcaattcaactggtacgtggacggggtggaggtgcacaatgcaaaaaccaaaccaagggaggaacagtttaattcaacatatagggttgtgtctgtgctgacggttctgcatcaggactggctgaacggaaaggaatacaagtgcaaggtgtccaacaaaggactgccaagctctatcgagaaaacaatctctaaggccaagggacaacctagagagccccaagtttacaccctgccaccatcacaggaagagatgaccaaaaatcaggtgagcttgacatgcctggtgaagggcttctaccctagcgatattgcggttgagtgggagtcaaatggccagcctgagaacaactataagactactcctcccgtgctggactccgacgggagctttttcctgtattccaggcttacagtcgataagagcagatggcaagaggggaatgtgttttcctgctccgtgatgcacgaggctctccataaccattatactcagaaaagtctctctctgtcactgggcaaa

SEQ ID NO: 84 - protein, 118 aa, Artificial Sequence; identical to SEQ ID NO: 32
  source: /note=“Description of Artificial Sequence: Synthetic polypeptide”

SEQ ID NO: 85 - DNA, 354 bp, Artificial Sequence
  source: /note=“Description of Artificial Sequence: Synthetic polynucleotide”
caggtgcaattggtgcagagcggagcagaggtcaaaaagcccggagcaagcgtgaaggtctcatgcaaagcaagcggatacacatttacatcatacaacatgcactgggtcaggcaggctccaggacagggactggagtggatcggggacatctaccctggacagggcgatactagctataatcagaagttcaaaggccgggccaccatgacagctgacaagtctactagtaccgtgtatatggaactgagctccctgcggtctgaagataccgcagtgtactattgcgccagagtcgggggggcatttcctatggattattgggggcaggggactctggtcactgtcagctca

SEQ ID NO: 86 - protein, 448 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGQGDTSYNQKFKGRATMTADKSTSTVYMELSSLRSEDTAVYYCARVGGAFPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - DNA, 1344 bp, Artificial Sequence
  source: /note=“Description of Artificial Sequence: Synthetic polynucleotide”
caggtgcaattggtgcagagcggagcagaggtcaaaaagcccggagcaagcgtgaaggtctcatgcaaagcaagcggatacacatttacatcatacaacatgcactgggtcaggcaggctccaggacagggactggagtggatcggggacatctaccctggacagggcgatactagctataatcagaagttcaaaggccgggccaccatgacagctgacaagtctactagtaccgtgtatatggaactgagctccctgcggtctgaagataccgcagtgtactattgcgccagagtcgggggggcatttcctatggattattgggggcaggggactctggtcactgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggccgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctggcagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 88 - protein, 111 aa, Artificial Sequence; identical to SEQ ID NO: 20
  source: /note=“Description of Artificial Sequence: Synthetic polypeptide”

SEQ ID NO: 89 - DNA, 333 bp, Artificial Sequence
  source: /note=“Description of Artificial Sequence: Synthetic polynucleotide”
gacatcgtcctgacacagtctcctgacagcctggcagtgagcctgggcgaaagggcaaccattaattgtagagcttccgagtccgtcgagtactatggcactagtctgatgcagtggtaccagcagaagccagggcagccccctaaactgctgatctatgcagctagcaacgtggagtccggagtcccagaccggttctctggaagtgggtcaggaaccgattttaccctgacaattagctccctgcaggcagaagacgtggccgtctactattgtcagcagagccgcaaggacccaagcacattcggaggggggaccaaagtggaaatcaag

SEQ ID NO: 90 - protein, 218 aa, Artificial Sequence; identical to SEQ ID NO: 22
  source: /note=“Description of Artificial Sequence: Synthetic polypeptide”

SEQ ID NO: 91 - DNA, 654 bp, Artificial Sequence
  source: /note=“Description of Artificial Sequence: Synthetic polynucleotide”
gacatcgtcctgacacagtctcctgacagcctggcagtgagcctgggcgaaagggcaaccattaattgtagagcttccgagtccgtcgagtactatggcactagtctgatgcagtggtaccagcagaagccagggcagccccctaaactgctgatctatgcagctagcaacgtggagtccggagtcccagaccggttctctggaagtgggtcaggaaccgattttaccctgacaattagctccctgcaggcagaagacgtggccgtctactattgtcagcagagccgcaaggacccaagcacattcggaggggggaccaaagtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 92 - protein, 118 aa, Artificial Sequence
  source: /note=“Description of Artificial Sequence: Synthetic polypeptide”
QVQLVQSGAEVKKPGASVKVSCKASGYTFTSYNMHWVRQAPGQGLEWMGDIYPGQGDTSYNQKFKGRVTMTRDTSTSTVYMELSSLRSEDTAVYYCARVGGAFPMDYWGQGTTVTVSS

SEQ ID NO: 93 - DNA, 354 bp, Artificial Sequence
  source: /note=“Description of Artificial Sequence: Synthetic polynucleotide”
caggtgcagctggtgcagtctggcgccgaagtgaagaaaccaggcgccagcgtgaaggtgtcctgcaaggccagcggctacacctttaccagctacaacatgcactgggtgcgccaggcccctggacagggactggaatggatgggcgacatctaccccggccagggcgacacctcctacaaccagaaattcaagggcagagtgaccatgacccgggacaccagcacctccaccgtgtacatggaactgagcagcctgcggagcgaggacaccgccgtgtactactgtgctagagtgggcggagccttccccatggactattggggccagggcaccaccgtgaccgtgagctca

SEQ ID NO: 94 - protein, 448 aa, Artificial Sequence
  source: /note=“Description of Artificial Sequence: Synthetic polypeptide”
QVQLVQSGAEVKKPGASVKVSCKASGYTFTSYNMHWVRQAPGQGLEWMGDIYPGQGDTSYNQKFKGRVTMTRDTSTSTVYMELSSLRSEDTAVYYCARVGGAFPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 1344 bp, Artificial Sequence
  source: /note=“Description of Artificial Sequence: Synthetic polynucleotide”
caggtgcagctggtgcagtctggcgccgaagtgaagaaaccaggcgccagcgtgaaggtgtcctgcaaggccagcggctacacctttaccagctacaacatgcactgggtgcgccaggcccctggacagggactggaatggatgggcgacatctaccccggccagggcgacacctcctacaaccagaaattcaagggcagagtgaccatgacccgggacaccagcacctccaccgtgtacatggaactgagcagcctgcggagcgaggacaccgccgtgtactactgtgctagagtgggcggagccttccccatggactattggggccagggcaccaccgtgaccgtgagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggccgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctggcagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 96 - protein, 111 aa, Artificial Sequence
  source: /note=“Description of Artificial Sequence: Synthetic polypeptide”
AIRLTQSPSSFSASTGDRVTITCRASESVEYYGTSLMQWYQQKPGKAPKLLIYAASNVESGVPSRFSGSGSGTDFTLTISSLQSEDFATYYCQQSRKDPSTFGGGTKVEIK

SEQ ID NO: 97 - DNA, 333 bp, Artificial Sequence
  source: /note=“Description of Artificial Sequence: Synthetic polynucleotide”
gccatcagactgacccagagccccagctcctttagcgccagcaccggcgacagagtgaccatcacctgtagagccagcgagagcgtggaatattacggcaccagcctgatgcagtggtatcagcagaagcccggcaaggcccccaagctgctgatctacgccgccagcaatgtggaaagcggcgtgcccagcagattcagcggctctggcagcggcaccgacttcaccctgacaatcagcagcctgcagagcgaggacttcgccacctactactgccagcagagccggaaggaccccagcacatttggcggaggcaccaaggtggaaatcaag

SEQ ID NO: 98 - protein, 218 aa, Artificial Sequence
  source: /note=“Description of Artificial Sequence: Synthetic polypeptide”
AIRLTQSPSSFSASTGDRVTITCRASESVEYYGTSLMQWYQQKPGKAPKLLIYAASNVESGVPSRFSGSGSGTDFTLTISSLQSEDFATYYCQQSRKDPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - DNA, 654 bp, Artificial Sequence
  source: /note=“Description of Artificial Sequence: Synthetic polynucleotide”
gccatcagactgacccagagccccagctcctttagcgccagcaccggcgacagagtgaccatcacctgtagagccagcgagagcgtggaatattacggcaccagcctgatgcagtggtatcagcagaagcccggcaaggcccccaagctgctgatctacgccgccagcaatgtggaaagcggcgtgcccagcagattcagcggctctggcagcggcaccgacttcaccctgacaatcagcagcctgcagagcgaggacttcgccacctactactgccagcagagccggaaggaccccagcacatttggcggaggcaccaaggtggaaatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 100 - protein, 118 aa, Artificial Sequence
  source: /note=“Description of Artificial Sequence: Synthetic polypeptide”
QVQLVQSGAEVKKPGESLKISCKGSGYTFTSYNMHWVRQMPGKGLEWMGDIYPGNGDTSYNQKFKGQVTISADKSISTVYLQWSSLKASDTAMYYCARVGGAFPMDYWGQGTTVTVSS

SEQ ID NO: 101 - DNA, 354 bp, Artificial Sequence
  source: /note=“Description of Artificial Sequence: Synthetic polynucleotide”
caggtgcaattggtacagtctggcgcagaagtaaagaaaccaggagagagtttgaaaatttcctgcaagggcagtgggtacacattcacgtcctacaatatgcactgggtgagacagatgccaggcaagggcctggagtggatgggagacatatacccaggcaatggagacacaagctataatcagaaattcaaaggacaggtgacgatctccgcagacaaatccatatctacggtctacctccagtggtcctcacttaaagcctccgacaccgccatgtactattgcgctcgggtaggtggcgcgtttccaatggactattggggccaagggaccacagtaaccgtcagctca

SEQ ID NO: 102 - protein, 448 aa, Artificial Sequence
  source: /note=“Description of Artificial Sequence: Synthetic polypeptide”
QVQLVQSGAEVKKPGESLKISCKGSGYTFTSYNMHWVRQMPGKGLEWMGDIYPGNGDTSYNQKFKGQVTISADKSISTVYLQWSSLKASDTAMYYCARVGGAFPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - DNA, 1344 bp, Artificial Sequence
  source: /note=“Description of Artificial Sequence: Synthetic polynucleotide”
caggtgcaattggtacagtctggcgcagaagtaaagaaaccaggagagagtttgaaaatttcctgcaagggcagtgggtacacattcacgtcctacaatatgcactgggtgagacagatgccaggcaagggcctggagtggatgggagacatatacccaggcaatggagacacaagctataatcagaaattcaaaggacaggtgacgatctccgcagacaaatccatatctacggtctacctccagtggtcctcacttaaagcctccgacaccgccatgtactattgcgctcgggtaggtggcgcgtttccaatggactattggggccaagggaccacagtaaccgtcagctcagctagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggccgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctggcagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 104 - protein, 111 aa, Artificial Sequence; identical to SEQ ID NO: 64
  source: /note=“Description of Artificial Sequence: Synthetic polypeptide”

SEQ ID NO: 105 - DNA, 333 bp, Artificial Sequence
  source: /note=“Description of Artificial Sequence: Synthetic polynucleotide”
gcaatacagttgacacagagtccttcaagtttgtccgcttccgttggcgaccgagtgacaatcacctgtagagcatccgagtcagtggagtattatggcactagcctgatgcagtggtatcagcaaaagccagggaaagccccaaagctgctgatatatgccgcgagtaacgtcgagtcaggggtgccatcaagattctccggttccgggtccggaaccgacttcacactgaccatctcttcccttcagccagaggacttcgctacgtacttttgccagcagtcacggaaagatccctctactttcggaggtgggacaaaagtcgaaattaaa

SEQ ID NO: 106 - protein, 218 aa, Artificial Sequence; identical to SEQ ID NO: 66
  source: /note=“Description of Artificial Sequence: Synthetic polypeptide”

SEQ ID NO: 107 - DNA, 654 bp, Artificial Sequence
  source: /note=“Description of Artificial Sequence: Synthetic polynucleotide”
gcaatacagttgacacagagtccttcaagtttgtccgcttccgttggcgaccgagtgacaatcacctgtagagcatccgagtcagtggagtattatggcactagcctgatgcagtggtatcagcaaaagccagggaaagccccaaagctgctgatatatgccgcgagtaacgtcgagtcaggggtgccatcaagattctccggttccgggtccggaaccgacttcacactgaccatctcttcccttcagccagaggacttcgctacgtacttttgccagcagtcacggaaagatccctctactttcggaggtgggacaaaagtcgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 108 - protein, 327 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 107 aa, Homo sapiens [sequence omitted]

SEQ ID NO: 110 - protein, 326 aa, Artificial Sequence
  source: /note=“Description of Artificial Sequence: Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 111 - protein, 330 aa, Unknown
  source: /note=“Description of Unknown: IgG1 wild type sequence”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 330 aa, Artificial Sequence
  source: /note=“Description of Artificial Sequence: Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - DNA, 354 bp, Artificial Sequence
  source: /note=“Description of Artificial Sequence: Synthetic polynucleotide”
caggtccagctggtccagagcggagcagaggtcaaaaagcccggagcaagcgtgaaggtctcatgcaaagcaagcggatacacatttacatcatacaacatgcactggatcaggcaggctccaggacagggactggagtggatcggggacatctaccctggaaacggcgatactagctataatcagaagttcaaaggccgggccaccctgacagctgacaagtctactagtaccgtgtatatggagctgagctccctgcggtctgaagataccgcagtgtactattgcgccagagtcgggggggcatttcctatggattattgggggcaggggactctggtcactgtctcctcc

SEQ ID NO: 116 - protein, 445 aa, Artificial Sequence
  source: /note=“Description of Artificial Sequence: Synthetic polypeptide”
QVQLVQSGAEVKKPGASVKVSCKASGYTFTSYNMHWIRQAPGQGLEWIGDIYPGNGDTSYNQKFKGRATLTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - DNA, 1335 bp, Artificial Sequence
  source: /note=“Description of Artificial Sequence: Synthetic polynucleotide”
caggtccagctggtccagagcggagcagaggtcaaaaagcccggagcaagcgtgaaggtctcatgcaaagcaagcggatacacatttacatcatacaacatgcactggatcaggcaggctccaggacagggactggagtggatcggggacatctaccctggaaacggcgatactagctataatcagaagttcaaaggccgggccaccctgacagctgacaagtctactagtaccgtgtatatggagctgagctccctgcggtctgaagataccgcagtgtactattgcgccagagtcgggggggcatttcctatggattattgggggcaggggactctggtcactgtctcctccgctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 118 - DNA, 333 bp, Artificial Sequence; identical to SEQ ID NO: 89
  source: /note=“Description of Artificial Sequence: Synthetic polynucleotide”

SEQ ID NO: 119 - DNA, 654 bp, Artificial Sequence
  source: /note=“Description of Artificial Sequence: Synthetic polynucleotide”
gacatcgtcctgacacagtctcctgacagcctggcagtgagcctgggcgaaagggcaaccattaattgtagagcttccgagtccgtcgagtactatggcactagtctgatgcagtggtaccagcagaagccagggcagccccctaaactgctgatctatgcagctagcaacgtggagtccggagtcccagaccggttctctggaagtgggtcaggaaccgattttaccctgacaattagctccctgcaggcagaagacgtggccgtctactattgtcagcagagccgcaaggacccaagcacattcggaggggggaccaaagtggaaatcaagcggactgttgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcaccggtgacaaagagcttcaacaggggagagtgt

SEQ ID NO: 120 - DNA, 354 bp, Artificial Sequence
  source: /note=“Description of Artificial Sequence: Synthetic polynucleotide”
caggtccagctggtccagagcggagcagaggtcaaaaagcccggagcaagcgtgaaggtctcatgcaaagcaagcggatacacatttacatcatacaacatgcactgggtcaggcaggctccaggacagggactggagtggatcggggacatctaccctggaaacggcgatactagctataatcagaagttcaaaggccgggccaccatgacagctgacaagtctactagtaccgtgtatatggagctgagctccctgcggtctgaagataccgcagtgtactattgcgccagagtcgggggggcatttcctatggattattgggggcaggggactctggtcactgtctcctcc

SEQ ID NO: 121 - protein, 445 aa, Artificial Sequence
  source: /note=“Description of Artificial Sequence: Synthetic polypeptide”
QVQLVQSGAEVKKPGASVKVSCKASGYTFTSYNMHWVRQAPGQGLEWIGDIYPGNGDTSYNQKFKGRATMTADKSTSTVYMELSSLRSEDTAVYYCARVGGAFPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 122 - DNA, 1335 bp, Artificial Sequence
  source: /note=“Description of Artificial Sequence: Synthetic polynucleotide”
caggtccagctggtccagagcggagcagaggtcaaaaagcccggagcaagcgtgaaggtctcatgcaaagcaagcggatacacatttacatcatacaacatgcactgggtcaggcaggctccaggacagggactggagtggatcggggacatctaccctggaaacggcgatactagctataatcagaagttcaaaggccgggccaccatgacagctgacaagtctactagtaccgtgtatatggagctgagctccctgcggtctgaagataccgcagtgtactattgcgccagagtcgggggggcatttcctatggattattgggggcaggggactctggtcactgtctcctccgctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 123 - DNA, 333 bp, Artificial Sequence
  source: /note=“Description of Artificial Sequence: Synthetic polynucleotide”
gacatcgtcctgacacagtctcctgacagcctggcagtgagcctgggcgaaagggcaaccattaattgtagagcttccgagtccgtcgagtactatggcactagtctgatgcagtggtaccagcagaagccagggcagccccctaaactgctgatctatgcagctagcaacgtggagtccggagtcccagaccggttctctggaagtgggtcaggaaccgattttaccctgacaattagctccctgcaggcagaagacgtggccgtctacttttgtcagcagagccgcaaggacccaagcacattcggaggggggaccaaagtggaaatcaag

SEQ ID NO: 124 - DNA, 654 bp, Artificial Sequence
  source: /note=“Description of Artificial Sequence: Synthetic polynucleotide”
gacatcgtcctgacacagtctcctgacagcctggcagtgagcctgggcgaaagggcaaccattaattgtagagcttccgagtccgtcgagtactatggcactagtctgatgcagtggtaccagcagaagccagggcagccccctaaactgctgatctatgcagctagcaacgtggagtccggagtcccagaccggttctctggaagtgggtcaggaaccgattttaccctgacaattagctccctgcaggcagaagacgtggccgtctacttttgtcagcagagccgcaaggacccaagcacattcggaggggggaccaaagtggaaatcaagcggactgttgctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcaccggtgacaaagagcttcaacaggggagagtgt

SEQ ID NO: 125 - DNA, 333 bp, Artificial Sequence; identical to SEQ ID NO: 105
  source: /note=“Description of Artificial Sequence: Synthetic polynucleotide”

SEQ ID NO: 126 - DNA, 654 bp, Artificial Sequence
  source: /note=“Description of Artificial Sequence: Synthetic polynucleotide”
gcaatacagttgacacagagtccttcaagtttgtccgcttccgttggcgaccgagtgacaatcacctgtagagcatccgagtcagtggagtattatggcactagcctgatgcagtggtatcagcaaaagccagggaaagccccaaagctgctgatatatgccgcgagtaacgtcgagtcaggggtgccatcaagattctccggttccgggtccggaaccgacttcacactgaccatctcttcccttcagccagaggacttcgctacgtacttttgccagcagtcacggaaagatccctctactttcggaggtgggacaaaagtcgaaattaaacgtacggtggcagctccgtctgttttcatctttccacctagcgacgagcaactcaaaagtggtacagcatccgtggtttgtctgctgaacaatttttaccccagggaggctaaggtccagtggaaagtcgataacgctcttcagtctggcaacagtcaggagagcgtcacagagcaggactctaaggatagcacttatagtctgtcctccacgctgacactgtctaaagcggattatgagaagcacaaggtttacgcctgtgaggtaacgcaccaaggactctcctccccagttaccaaatctttcaacagaggagaatgt

SEQ ID NO: 127 - DNA, 333 bp, Artificial Sequence
  source: /note=“Description of Artificial Sequence: Synthetic polynucleotide”
gagattgttcttacgcaaagtcccgccacacttagtttgtcaccaggagagcgcgccaccctgagctgcagagcttcagagagtgtggaatactacggcacatccctgatgcagtggtatcagcagaaaccaggacaggctcctcggctgctgatctacgcagccagcaacgtcgagtccggcattccagccagattttctgggtcaggatctggaactgactttacactgacaatctccagcctggaacccgaggacattgctgtgtatttttgtcaacagtcccggaaggaccccagtacctttggaggtggaaccaaggtagagataaag

SEQ ID NO: 128 - DNA, 654 bp, Artificial Sequence
  source: /note=“Description of Artificial Sequence: Synthetic polynucleotide”
gagattgttcttacgcaaagtcccgccacacttagtttgtcaccaggagagcgcgccaccctgagctgcagagcttcagagagtgtggaatactacggcacatccctgatgcagtggtatcagcagaaaccaggacaggctcctcggctgctgatctacgcagccagcaacgtcgagtccggcattccagccagattttctgggtcaggatctggaactgactttacactgacaatctccagcctggaacccgaggacattgctgtgtatttttgtcaacagtcccggaaggaccccagtacctttggaggtggaaccaaggtagagataaagcgtacggtggcagctccgtctgttttcatctttccacctagcgacgagcaactcaaaagtggtacagcatccgtggtttgtctgctgaacaatttttaccccagggaggctaaggtccagtggaaagtcgataacgctcttcagtctggcaacagtcaggagagcgtcacagagcaggactctaaggatagcacttatagtctgtcctccacgctgacactgtctaaagcggattatgagaagcacaaggtttacgcctgtgaggtaacgcaccaaggactctcctccccagttaccaaatctttcaacagaggagaatgt

SEQ ID NO: 129-829 - skipped (701 entries)

SEQ ID NO: 830 - protein, 114 aa, Artificial Sequence
  source: /note=“Description of Artificial Sequence: Synthetic polypeptide”
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 831 - protein, 108 aa, Artificial Sequence
  source: /note=“Description of Artificial Sequence: Synthetic polypeptide”
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 832 - protein, 120 aa, Artificial Sequence
  source: /note=“Description of Artificial Sequence: Synthetic polypeptide”
EVQVLESGGGLVQPGGSLRLYCVASGFTFSGSYAMSWVRQAPGKGLEWVSAISGSGGSTYYADSVKGRFTISRDNSKNTLYLQMNSLRAEDTAVYYCAKKYYVGPADYWGQGTLVTVSSG

SEQ ID NO: 833 - protein, 113 aa, Artificial Sequence
  source: /note=“Description of Artificial Sequence: Synthetic polypeptide”
DIVMTQSPDSLAVSLGERATINCKSSQSVLYSSNNKNYLAWYQHKPGQPPKLLIYWASTRESGVPDRFSGSGSGTDFTLTISSLQAEDVAVYYCQQYYSSPLTFGGGTKIEVK

(19) United States
(12) Patent Application Publication
(10) Pub. No.: US 2022/0306737 A1
(43) Pub. Date: Sep. 29, 2022

(54) USE OF HIGH-AFFINITY, LIGAND-BLOCKING, HUMANIZED ANTI-T-CELL IMMUNOGLOBULIN DOMAIN AND MUCIN DOMAIN-3 (TIM-3) IGG4 ANTIBODY FOR THE TREATMENT OF MYELOFIBROSIS

(71) Applicant: Novartis AG, Basel (CH)

(72) Inventors: Asa Eliasson, Basel (CH); Hans Menssen, Basel (CH); K Gary J Vanasse, Chestnut Hill, MA (US); Monika Wroclawska, Basel (CH)

(21) Appl. No.: 17/642,340

(22) PCT Filed: Sep. 14, 2020
(86) PCT No.: PCT/IB2020/058516

§ 371 Date: Mar. 11, 2022

Related U.S. Application Data
(60) Provisional application No. 62/900,938, filed on Sep. 16, 2019.

(51) Int. Cl. C07K 16/28; A61P 35/00; A61P 19/00; A61P 7/00; A61K 39/395; A61K 31/519

(52) U.S. Cl. CPC C07K 16/2803; A61P 35/00; A61P 19/00; A61P 7/00; A61K 39/39541; A61K 31/519; A61K 2039/505

(57) ABSTRACT

The invention relates to the use of an anti-TIM-3 antibody molecule in the treatment of myelofibrosis (MF). The invention also relates to a pharmaceutical combination comprising a) an anti-TIM-3 antibody molecule and b) at least one further therapeutic agent, preferably ruxolitinib or a pharmaceutically acceptable salt thereof.

The present invention relates to uses of a high-affinity, ligand-blocking, humanized anti-T-cell immunoglobulin domain and mucin domain-3 (TIM-3) IgG4 antibody and combination thereof for the treatment of myelofibrosis.

### FIELD OF THE INVENTION

The invention relates to the use of a high-affinity, ligand-blocking, humanized anti-T-cell immunoglobulin domain and mucin domain-3 (TIM-3) IgG4 antibody in the treatment of myelofibrosis (MF). The invention also relates to a pharmaceutical combination comprising a TIM-3) IgG4 antibody and b) at least one further therapeutic agent.

### BACKGROUND OF THE INVENTION

Myeloproliferative neoplasms (MPNs) are a unique and heterogeneous group of hemopathies characterized by proliferation and accumulation of mature myeloid cells, including myelofibrosis (MF), essential thrombocythemia (ET) and polycythemia vera (PV). Importantly, MF is the most severe form of Philadelphia chromosome-negative (i.e. BCR-ABL1-negative) myeloproliferative neoplasms, with a prevalence estimated to be 2.2 per 100,000 population. Myelofibrosis (MF) can present as a de novo disorder (PMF) or evolve from previous PV or ET (PPV-MF or PET-MF). The range of reported frequencies for post-PV MF are 4.9-6% at 10 years and 6-14% at 15 years, respectively, and 0.8-4.9% for post-ET MF at 10 years and 4-11% at 15 years, respectively (S Cerquozzi and A Tefferi, Blood Cancer Journal (2015) 5, e366).

Regardless of whether MF developed from PV, ET or as a primary disorder, it is characterized by a clonal stem cell proliferation associated with production of elevated levels of several inflammatory and proangiogenic cytokines resulting in a bone marrow stromal reaction that includes varying degrees of reticulin and/or collagen fibrosis, osteosclerosis and angiogenesis, some degree of megakaryocyte atypia and a peripheral blood smear showing a leukoerythroblastic pattern with varying degrees of circulating progenitor cells. The abnormal bone marrow milieu results in release of hematopoietic stem cells into the blood, extramedullary hematopoiesis, and organomegaly at these sites. Clinically, MF is characterized by progressive anemia, leukopenia or leukocytosis, thrombocytopenia or thrombocythemia and multi-organ extramedullary hematopoiesis, which most prominently involves the spleen leading to massive splenomegaly, severe constitutional symptoms, a hypermetabolic state, cachexia, and premature death.

A considerable number of cytokine and growth factor receptors utilize non-receptor tyrosine kinases, the Janus kinases (JAK), to transmit extracellular ligand binding into an intracellular response. For example, erythropoietin, thrombopoietin and granulocyte monocyte colony stimulating factor are all known to signal through receptors that utilize JAK2. JAK activate a number of downstream pathways implicated in proliferation and survival, including the STATs (signal transducers and activators of transcription), a family of important latent transcription factors.

Myelofibrosis is now known to be a clonal stem cell disease characterized by molecular (JAK2V617F, MPLW515L/K) and cytogenetic (13q-,20q-) markers (Pikman Y, Lee B H, Mercher T, et al. PLoS Med. 2006; 3(7):e270; Scott L M, Tong W, Levine R L, et al. N Engl J Med. 2007; 356:459-468). The JAK2V617F mutation has been identified in over 95% of patients with PV and approximately 50% of patients with ET and PMF. Furthermore, in a preclinical setting, animal studies have demonstrated that this mutation can lead to an MF-like syndrome. The JAK2V617F mutation alters the JAK2 tyrosine kinase making it constitutively active. As a result, polycythemia, thrombocythemia and leukocytosis can develop independently from growth factor regulation. Even in patients lacking a confirmed JAK2 mutation, the detection of STAT activation suggests dysregulated JAK activity. In fact, regardless of the mutational status of JAK2, the malignant cells appear to retain their responsiveness to JAK activating cytokines and/or growth factors; hence, they may benefit from JAK inhibition. Although several JAK inhibitors, including ruxolitinib (brand name Jakavi) have been approved for the treatment of MF, they have only demonstrated effect in treatment of symptoms. Progression of the disease is not halted and eventually patients may die prematurely.

Patients with MF have shortened survival (median survival is 6.5 years) and greatly compromised quality of life (QoL). Contributing factors for shortened survival include leukemic transformation and thrombohemorrhagic complications and for the compromised quality of life severe anemia (often requiring red blood cell (RBC) transfusions), symptomatic enlargement of the spleen and liver, substantial MF-associated symptoms burden (MF-SB), and cachexia (Tefferi and Barbui 2019).

The only potential curative treatment for MF is allogeneic hematopoietic stem cell transplantation (ASCT), for which the great majority of patients are ineligible. Therefore, treatment options remain primarily palliative and aimed at controlling disease symptoms, complications and improving the patient's QoL. The therapeutic landscape of MF has changed with the discovery of the V617F mutation of the Janus kinase JAK2 gene present in 60% of patients with PMF or PET-MF and in 95% of patients with PPV-MF, triggering the development of molecular targeted therapy for MF (Cervantes 2014). JAK play an important role in signal transduction following cytokine and growth factor binding to their receptors. Aberrant activation of JAK has been associated with increased malignant cell proliferation and survival (Valentino and Pierre 2006). JAK activate a number of downstream signaling pathways implicated in the proliferation and survival of malignant cells including members of the Signal Transducer and Activator of Transcriptions (STAT) family of transcription factors.

JAK inhibitors were developed to target JAK2 thereby inhibiting JAK signaling. Ruxolitinib, as all agents of this class, mainly inhibits dysregulated JAK-STAT signaling present in all MF patients irrespective of their JAK2 mutational status, but is not selective for the mutated JAK2, which explains its efficacy in both JAK2-positive and -negative MF. Ruxolitinib is highly effective in reducing the spleen size and controlling the symptoms of MF, with this resulting in a marked improvement in the patient's QoL (Cervantes et al 2016). Ruxolitinib is the only JAK inhibitor that has been granted a marketing authorization, as a single agent, for the treatment of patients with PMF, PPV-MF or PET-MF and for the treatment of patients with PV who are resistant to or intolerant to hydroxyurea. Ruxolitinib is the only approved pharmacological treatment for MF patients with splenomegaly and/or clinical symptoms and is considered standard of care (SoC). Although ruxolitinib has changed the treatment paradigm of MF patients, there is no clear indication of its disease-modifying effect (Cervantes 2014) and therapy-related anemia is often an anticipated downside (Naymagon and Mascarenhas 2017, Mead et al 2015).

Activation of naive CD4+ T helper cells results in the development of at least two distinct effector populations, Th1 cells and Th2 cells. See U.S. Pat. No. 7,470,428, Mosmann T R et al. (1986) J Immunol 136:2348-57; Mosmann T R et al. (1996) Immunol Today 17:138-46; Abbas A K et al. (1996) Nature 383:787-793. Th1 cells produce cytokines (e.g., interferon gamma, interleukin-2, tumor necrosis factor alpha, and lymphotoxin) which are commonly associated with cell-mediated immune responses against intracellular pathogens, delayed-type hypersensitivity reactions (Sher A et al. (1992) Annu Rev Immunol 10:385-409), and induction of organ-specific autoimmune diseases. Liblau R S et al. (1995) Immunol Today 16:34-38. Th2 cells produce cytokines (e.g., IL-4, IL-10, and IL-13) that are crucial for control of extracellular helminthic infections and promote atopic and allergic diseases. Sher A et al. (1992) Annu Rev Immunol 10:385-409. In addition to their distinct roles in disease, the Th1 and Th2 cells cross-regulate each other's expansion and functions. Thus, preferential induction of Th2 cells inhibits autoimmune diseases (Kuchroo V K et al. (1995) Cell 80:707-18; Nicholson L B et al. (1995) Immunity 3:397-405), and predominant induction of Th1 cells can regulate induction of asthma, atopy and allergies. Lack G et al. (1994) J Immunol 152:2546-54; Hofstra C L et al. (1998) J Immunol 161:5054-60.

TIM-3 is a transmembrane receptor protein that is expressed, e.g., on Th1 (T helper 1) CD4+ cells and cytotoxic CD8+ T cells that secrete IFN-γ. TIM-3 is generally not expressed on naïve T cells but rather upregulated on activated, effector T cells. TIM-3 has a role in regulating immunity and tolerance in vivo (see Hastings et al., Eur J Immunol. 2009 September; 39(9):2492-501). There is a need in the art for new molecules that regulate TIM-3 function and the function of TIM-3 expressing cells.

WO/2017/019897 discloses combination an inhibitor of TIM-3 in combination with various other therapeutic agents, including JAK inhibitor such as ruxolitinib for the treatment of various cancers. However, it does not specifically disclose the treatment of myelofibrosis.

There remains a high unmet medical need to finding new and efficacious treatment options for advancing the treatment of myelofibrosis.

### SUMMARY OF THE INVENTION

It is an object of the present invention to provide for a medicament for the treatment of myelofibrosis. The present invention is based on the inventors' surprising finding that an antibody to TIM-3 is useful in the treatment of myelofibrosis in a subject.

The present invention is also based on finding that, an antibody to TIM-3 in combination with at least one further therapeutic agent is useful in the treatment of myelofibrosis in a subject.

In an embodiment, the antibody to TIM-3 is in combination with a JAK inhibitor.

In an embodiment, the JAK inhibitor is a JAK1/2 inhibitor.

In an embodiment, the JAK inhibitor is ruxolitinib or pharmaceutically acceptable salt thereof.

In an embodiment, the antibody to TIM-3 and the JAK inhibitor are in the same formulation.

In another embodiment, an antibody to TIM-3 and the JAK inhibitor are in separate formulations.

In a further embodiment, the pharmaceutical combination is for simultaneous or sequential administration.

### BRIEF DESCRIPTION OF THE TABLES

Each of the Tables is described herein in more detail.

Table 1 summarizes the sequences of the murine anti-TIM-3 antibody, ABTIM3.

Table 2 depicts the amino acid sequences of ABTIM3 heavy chain variable domain and light chain variable domain.

Table 3 depicts the amino acid sequences of ABTIM3 heavy chain CDRs and light chain CDRs.

Table 4 is a summary of the amino acid and nucleotide sequences for the murine and humanized anti-TIM-3 antibody molecules. The antibody molecules include murine ABTIM3 and humanized anti-TIM-3 antibodies: ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, and ABTIM3-hum23. The amino acid and nucleotide sequences of the heavy and light chain CDRs, the amino acid and nucleotide sequences of the heavy and light chain variable regions, and the amino acid and nucleotide sequences of the heavy and light chains are shown in this Table.

Table 5 depicts the constant region amino acid sequences of human IgG heavy chains and human kappa light chain.

### DETAILED DESCRIPTION OF THE INVENTION

Certain terms used herein are described below. Compounds or biological agents of the present invention are described using standard nomenclature. Unless defined otherwise, all technical and scientific terms used herein have the same meaning as is commonly understood by one of skill in the art to which this invention belongs.

The term “combination,” “therapeutic combination,” or “pharmaceutical combination” as used herein refer to either a fixed combination in one dosage unit form, or non-fixed combination, or a kit of parts for the combined administration where two or more therapeutic agents may be administered independently, at the same time or separately within time intervals, especially where these time intervals allow that the combination partners show a cooperative, e.g., synergistic, effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

The term “JAK inhibitor” as used herein refers to a compound that selectively targets, decreases, or inhibits at least one activity of JAK.

The term “pharmaceutical composition” is defined herein to refer to a mixture or solution containing at least one therapeutic agent to be administered to a subject, e.g., a mammal or human, in order to prevent or treat a particular disease or condition affecting the mammal.

The term “pharmaceutically acceptable” as used herein refers to those compounds, biological agents (e.g., antibodies), materials, compositions and/or dosage forms, which are, within the scope of sound medical judgment, suitable for contact with the tissues of a warm-blooded animal, e.g., a mammal or human, without excessive toxicity, irritation, allergic response, and other problem complications commensurate with a reasonable benefit/risk ratio.

The terms “fixed combination,” “fixed dose,” and “single formulation” as used herein refers to a single carrier or vehicle or dosage form formulated to deliver an amount, which is jointly therapeutically effective for the treatment or prevention of cancer, of both therapeutic agents to a patient. The single vehicle is designed to deliver an amount of each of the agents, along with any pharmaceutically acceptable carriers or excipients. In some embodiments, the vehicle is a tablet, capsule, pill, or a patch. In other embodiments, the vehicle is a solution or a suspension.

The term “non-fixed combination,” “kit of parts,” and “separate formulations” means that at least one of the active ingredients is administered to a patient as a separate entity either simultaneously, concurrently, or sequentially with no specific time limits, wherein such administration provides therapeutically effective levels of the two active ingredients agents in the body of the subject in need thereof. The latter also applies to cocktail therapy, e.g., the administration of three or more active ingredients.

The term “unit dose” is used herein to mean simultaneous administration of both agents together, in one dosage form, to the patient being treated. In some embodiments, the unit dose is a single formulation. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, injections, infusions, patches, or the like, administered to the patient at the same time.

An “oral dosage form” includes a unit dosage form prescribed or intended for oral administration.

The term “treating” or “treatment” as used herein comprises a treatment relieving, reducing, or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease), and/or reduce the risk of developing or worsening a disease. The term “protect” is used herein to mean prevent, delay, or treat, or all, as appropriate, development, continuance or aggravation of a disease in a subject, e.g., a mammal or human. The term “prevent”, “preventing” or “prevention” as used herein comprises the prevention of at least one symptom associated with or caused by the state, disease or disorder being prevented.

The term “pharmaceutically effective amount,” “therapeutically effective amount,” or “clinically effective amount” of a combination of therapeutic agents is an amount sufficient to provide an observable or clinically significant improvement over the baseline clinically observable signs and symptoms of the disorders treated with the combination.

The term “jointly therapeutically active” or “joint therapeutic effect” as used herein means that the therapeutic agents can be given separately (in a chronologically staggered manner, especially a sequence-specific manner) in such time intervals that they prefer, in the warm-blooded animal, especially human, to be treated, still show an (preferably synergistic) interaction (joint therapeutic effect). Whether this is the case can, inter alia, be determined by following the blood levels of the compounds, showing that both compounds are present in the blood of the human to be treated at least during certain time intervals.

The term “subject” or “patient” as used herein is intended to include animals, which are capable of suffering from or afflicted with a cancer or any disorder involving, directly or indirectly, a cancer. Examples of subjects include mammals, e.g., humans, apes, monkeys, dogs, cows, horses, pigs, sheep, goats, cats, mice, rabbits, rats, and transgenic non-human animals. In an embodiment, the subject is a human, e.g., a human suffering from, at risk of suffering from, or potentially capable of suffering from cancers.

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, biological agents, salts, and the like, this is taken to mean also a single compound, salt, or the like.

The terms “about” or “approximately” are generally understood by persons knowledgeable in the relevant subject area, but in certain circumstances can mean within 20%, within 10%, or within 5% of a given value or range. Alternatively, especially in biological systems, the term “about” means within about a log (i.e., an order of magnitude) or within a factor of two of a given value.

Antibody Molecules to TIM-3

In one embodiment, the TIM-3 inhibitor is an anti-TIM-3 antibody molecule as described in US Patent Application Publication No. 2015/0218274 (U.S. Ser. No. 14/610,837), filed Jan. 30, 2015, entitled “Antibody Molecules to TIM-3 and Uses Thereof,” incorporated by reference in its entirety. In some embodiments, the anti-TIM-3 antibody is MBG453, which is disclosed in US Patent Application Publication No. 2015/0218274.

In certain embodiments, the anti-TIM-3 antibody molecule (e.g., an isolated or recombinant antibody molecule) has one or more (e.g., 2, 3, 4, 5, 6, 7, 8, 9, 10, 11, 12, 13, 14, 15, 16 or all) of the following properties (a), (b), (c), (d), (e), (f), (g), (h), (i), (j), (k), (l), (m), (n), (o), (p) or (q):

(a) binds to TIM-3, e.g., human TIM-3, with high affinity, e.g., with a dissociation constant (KD) of less than about 100 nM, typically about 10 nM, and more typically, about 1-0.1 nM or stronger, e.g., less than about 0.2, 0.16, 0.15, 0.1, 0.075, 0.05, or 0.042 nM,

(b) binds substantially to a non-human primate TIM-3, e.g., cynomolgus TIM-3, with a dissociation constant (KD) of less than about 100 nM, typically about 10 nM, and more typically, about 3-0.3 nM or stronger, e.g., 1-0.1 nM or stronger, e.g., less than about 1 nM, 0.75 nM, or 0.68 nM,

(c) inhibits binding of TIM-3 to a TIM-3 ligand, e.g., phosphatidylserine (PtdSer), HMGB1, or CEACAM-1,

(d) enhances IFN-gamma and/or TNF-alpha secretion and/or proliferation in T cells, e.g., CD4+ or CD8+ T cells, e.g., in CD4+ T cells that were stimulated with anti-CD3/CD28 in the presence of IL-12 or in T cell-DC autologous culture assays with anti-CD3/CD28 stimulation,

(e) enhances cytotoxic NK (natural killer) cell activity against a target cell (e.g., K562 cells), e.g., in an in vitro assay,

(f) enhances capacity of macrophages or antigen presenting cells to stimulate a T cell response, e.g., increasing IL-12 secretion of antigen presenting cells,

(g) binds specifically to an epitope on TIM-3, e.g., the same or similar epitope as the epitope recognized by an antibody molecule described herein, e.g., a murine or humanized anti-TIM-3 antibody molecule as described herein, e.g., an antibody molecule of Tables 1-4,

(h) shows the same or similar binding affinity or specificity, or both, as an antibody molecule of Tables 1-4,

(i) shows the same or similar binding affinity or specificity, or both, as an antibody molecule (e.g., an heavy chain variable region and light chain variable region) described in Tables 1-4,

(j) shows the same or similar binding affinity or specificity, or both, as an antibody molecule (e.g., an heavy chain variable region and light chain variable region) comprising an amino acid sequence shown in Tables 1-4,

(k) inhibits, e.g., competitively inhibits, the binding of a second antibody molecule to TIM-3 wherein the second antibody molecule is an antibody molecule described herein, e.g., an antibody molecule chosen from Tables 1-4,

(l) binds the same (or substantially the same) or an overlapping (or substantially overlapping) epitope with a second antibody molecule to TIM-3, wherein the second antibody molecule is an antibody molecule described herein, e.g., an antibody molecule chosen from Tables 1-4,

(m) competes for binding, and/or binds the same (or substantially the same) or overlapping (or substantially overlapping) epitope, with a second antibody molecule to TIM-3, wherein the second antibody molecule is an antibody molecule described herein, e.g., an antibody molecule chosen from Tables 1-4, e.g., as determined by the methods described in Example 11,

(n) has one or more biological properties of an antibody molecule described herein, e.g., an antibody molecule chosen from Tables 1-4,

(o) has one or more pharmacokinetic properties of an antibody molecule described herein, e.g., an antibody molecule chosen from Tables 1-4,

(p) modulates (e.g., enhances or inhibits) one or more activities of TIM-3, e.g., results in one or more of: enhancing IFN-gamma and/or TNF-alpha secretion in T cells; enhancing proliferation in T cells, e.g., CD4+ or CD8+ T cells; enhancing NK cell cytotoxic activity; reducing suppressor activity of regulatory T cells (Tregs); or increasing immune stimulation properties of macrophages and/or antigen presenting cells, e.g., increasing cytokine secretion, e.g., IL-12 secretion; or

(q) binds to one or more residues within: the two residues adjacent to the N-terminus of the A strand (residues Val24 and Glu25 in human TIM-3), the BC loop, the CC′ loop, the F strand, the FG loop, and the G strand of TIM-3, or one or more residues within a combination of two, three, four, five or all of: the two residues adjacent to the N-terminus of the A strand (residues Val24 and Glu25 in human TIM-3), the BC loop, the CC′ loop, the F strand, the FG loop, and the G strand of TIM-3, e.g., wherein the binding is assayed using ELISA or Biacore.

In some embodiments, the antibody molecule binds to TIM-3 with high affinity, e.g., with a KD that is at least about 10%, 20%, 30%, 40%, 50%, 60%, 70%, 80% or 90% lower than the KD of a murine anti-TIM-3 antibody molecule, e.g., a murine anti-TIM-3 antibody molecule described herein.

In some embodiments, the expression level of the anti-TIM-3 antibody molecule is higher, e.g., at least about 0.5, 1, 2, 3, 4, 5, 6, 7, 8, 9 or 10-fold higher, than the expression level of a murine antibody molecule, e.g., a murine or chimeric anti-TIM-3 antibody molecule described herein. In some embodiments, the antibody molecule is expressed in mammalian cells, e.g., rodent cells.

In some embodiments, the anti-TIM-3 antibody molecule reduces one or more activities of TIM-3 with an IC50 (concentration at 50% inhibition) that is lower, e.g., at least about 10%, 20%, 30%, 40%, 50%, 60%, 70%, 80% or 90% lower, than the IC50 of a murine anti-TIM-3 antibody molecule, e.g., a murine anti-TIM-3 antibody molecule described herein. In some embodiments, the TIM-3 activity is the binding of TIM-3 to one, two or more (e.g., one, two, three, four or all) of the TIM-3 ligands described herein, e.g., one, two or more (all) of PtdSer, CEACAM-1, or HMGB1.

In some embodiments, the anti-TIM-3 antibody molecule interacts with, e.g., binds to, a TIM-3 surface (e.g., one, two, three, five, eight, ten, fifteen, or more continuous or discontinuous (e.g., noncontiguous) amino acid residues chosen from Val24, Glu25, Thr41, Gly56, Ala57, Cys58, Pro59, Val60, Phe61, Glu121, Lys122, Phe123, Asn124, Leu125, Lys126, and/or Leu127.

In some embodiments, the anti-TIM-3 antibody molecule interacts with, e.g., binds to, a TIM-3 surface (e.g., one, two, three, five, eight, ten, fifteen, twenty, twenty-one, twenty-five, or more continuous or discontinuous (e.g., noncontiguous) amino acid residues chosen from Val24, Glu25, Tyr26, Phe39, Tyr40, Thr41, Gly56, Ala57, Cys58, Pro59, Val60, Phe61, Ser105, Gly106, Ile107, Asn119, Asp120, Glu121, Lys122, Phe123, Asn124, Leu125, Lys126, Leu127, and/or Val128.

In some embodiments, the anti-TIM-3 antibody molecule interacts with, e.g., binds to, a TIM-3 surface (e.g., one, two, three, five, eight, ten, fifteen, twenty, twenty-one, twenty-five, or more continuous or discontinuous (e.g., noncontiguous) amino acid residues chosen from Glu23, Val24, Glu25, Tyr26, Thr41, Pro42, Ala43, Ala44, Pro45, Gly46, Asn47, Leu48, Val49, Pro50, Val51, Cys52, Trp53, Gly54, Lys55, Gly56, Ala57, Cys58, Pro59, Val60, Phe61, Glu121, Lys122, Phe123, Asn124, Leu125, Lys126 and/or Leu127.

In some embodiments, the anti-TIM-3 antibody molecule interacts with, e.g., binds to, a TIM-3 surface (e.g., one, two, three, five, eight, ten, fifteen, twenty, twenty-one, twenty-five, or more continuous or discontinuous (e.g., noncontiguous) amino acid residues chosen from Val24, Glu25, Tyr26, Phe39, Tyr40, Thr41, Pro42, Ala43, Ala44, Pro45, Gly46, Asn47, Leu48, Val49, Pro50, Val51, Cys52, Trp53, Gly54, Lys55, Gly56, Ala57, Cys58, Pro59, Val60, Phe61, Ser105, Gly106, Ile107, Asn119, Asp120, Glu121, Lys122, Phe123, Asn124, Leu125, Lys126, Leu127, and/or Val128.

In other embodiments, the anti-TIM-3 antibody molecule competes with CEACAM-1 for binding to TIM-3. In one embodiment, the anti-TIM-3 antibody molecule interacts, e.g., binds to, one, two, or more (all) of Cys58, Asn119 and Lys122 of TIM-3, e.g., displaces or competes CEACAM-1 for binding to these residues. In one embodiment, the anti-TIM-3 antibody molecule reduces or blocks the formation of a hydrogen bond between Lys122 of TIM-3 and Asn42 of CEACAM-1, e.g., by at least about 10%, 20%, 30%, 40%, 50%, 60%, 70%, 80% or 90%, compared to the formation of a hydrogen bond between between Lys122 of TIM-3 and Asn42 of CEACAM-1 in the absence of the anti-TIM-3 antibody molecule.

In another embodiment, the anti-TIM-3 antibody molecule interacts with, e.g., binds to, a PtdSer-binding loop of TIM-3. In one embodiment, the anti-TIM-3 antibody molecule interacts with, e.g., binds to, at least two PtdSer-binding loops of TIM-3, e.g., the FG loop and CC′ loop of TIM-3 (e.g., a metal ion-dependent ligand binding site (MILIBS)). For example, the carboxyl group of PtdSer can bind to the CC′ loop of TIM-3 and the amino group of PtdSer can bind to the FG loop of TIM-3. In one embodiment, the anti-TIM-3 antibody molecule reduces or prevents PtdSer-mediated membrane penetration of TIM-3.

In another embodiment, the anti-TIM-3 antibody molecule competes with HMGB1 for binding to TIM-3. E.g., it reduces binding of HMGB1 to residue 62 of TIM-3 (Q in mouse, E in human TIM-3), e.g., by at least about 10%, 20%, 30%, 40%, 50%, 60%, 70%, 80% or 90%, compared to the binding of HMGB1 to residue 62 of TIM-3 in the absence of the anti-TIM-3 antibody molecule.

In yet another embodiment, the anti-TIM-3 antibody molecule does not compete with a Galectin-9 (Gal-9) ligand for binding to TIM-3.

In some embodiments, the anti-TIM-3 antibody molecule has improved stability, e.g., at least about 0.5, 1, 2, 3, 4, 5, 6, 7, 8, 9 or 10-fold more stable in vivo or in vitro, than a murine or humanized anti-TIM-3 antibody molecule, e.g., a murine or humanized anti-TIM-3 antibody molecule described herein.

In some embodiments, the anti-TIM-3 antibody molecule comprises at least one antigen-binding region, e.g., a variable region or an antigen-binding fragment thereof, from an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In certain embodiments, the anti-TIM-3 antibody molecule comprises at least one, two, three, or four variable regions from an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In some embodiments, the anti-TIM-3 antibody molecule comprises at least one or two heavy chain variable regions from an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In certain embodiments, the anti-TIM-3 antibody molecule comprises at least one or two light chain variable regions from an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In one embodiment, the anti-TIM-3 antibody molecule includes a heavy chain constant region for an IgG4, e.g., a human IgG4. In another embodiment, the human IgG4 includes a substitution (e.g., a Ser to Pro substitution) at position 228 according to EU numbering or at position 108 of SEQ ID NO: 108 or 110. In still another embodiment, the anti-TIM-3 antibody molecule includes a heavy chain constant region for an IgG1, e.g., a human IgG1. In one embodiment, the human IgG1 includes a substitution (e.g., an Asn to Ala substitution) at position 297 according to EU numbering or at position 180 of SEQ ID NO: 112. In one embodiment, the human IgG1 includes a substitution (e.g., an Asp to Ala substitution) at position 265 according to EU numbering or at position 148 of SEQ ID NO: 113, a substitution (e.g., a Pro to Ala substitution) at position 329 according to EU numbering or at position 212 of SEQ ID NO: 113, or both. In one embodiment, the human IgG1 includes a substitution (e.g., a Leu to Ala substitution) at position 234 according to EU numbering or at position 117 of SEQ ID NO: 114, a substitution (e.g., a Leu to Ala substitution) at position 235 according to EU numbering or at position 118 of SEQ ID NO: 114, or both. In one embodiment, the heavy chain constant region comprises an amino sequence set forth in Table 1-5, or a sequence substantially identical (e.g., at least 80%, 85%, 90%, 92%, 95%, 97%, 98%, 99% or higher identical) thereto.

In yet another embodiment, the anti-TIM-3 antibody molecule includes a kappa light chain constant region, e.g., a human kappa light chain constant region. In one embodiment, the light chain constant region comprises an amino sequence set forth in Table 1-5, or a sequence substantially identical (e.g., at least 80%, 85%, 90%, 92%, 95%, 97%, 98%, 99% or higher identical) thereto.

In another embodiment, the anti-TIM-3 antibody molecule includes a heavy chain constant region for an IgG4, e.g., a human IgG4, and a kappa light chain constant region, e.g., a human kappa light chain constant region, e.g., a heavy and light chain constant region comprising an amino sequence set forth in Table 1-5, or a sequence substantially identical (e.g., at least 80%, 85%, 90%, 92%, 95%, 97%, 98%, 99% or higher identical) thereto. In yet another embodiment, the anti-TIM-3 antibody molecule includes a heavy chain constant region for an IgG1, e.g., a human IgG1, and a kappa light chain constant region, e.g., a human kappa light chain constant region, e.g., a heavy and light chain constant region comprising an amino sequence set forth in Table 1-5, or a sequence substantially identical (e.g., at least 80%, 85%, 90%, 92%, 95%, 97%, 98%, 99% or higher identical) thereto. In one embodiment, the human IgG1 includes a substitution at position 297 according to EU numbering (e.g., an Asn to Ala substitution). In one embodiment, the human IgG1 includes a substitution at position 265 according to EU numbering, a substitution at position 329 according to EU numbering, or both (e.g., an Asp to Ala substitution at position 265 and/or a Pro to Ala substitution at position 329). In one embodiment, the human IgG1 includes a substitution at position 234 according to EU numbering, a substitution at position 235 according to EU numbering, or both (e.g., a Leu to Ala substitution at position 234 and/or a Leu to Ala substitution at position 235).

In another embodiment, the anti-TIM-3 antibody molecule includes a heavy chain variable domain and a constant region, a light chain variable domain and a constant region, or both, comprising the amino acid sequence of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In some embodiments, the anti-TIM-3 antibody molecule includes at least one, two, or three complementarity determining regions (CDRs) from a heavy chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4, or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In some embodiments, the anti-TIM-3 antibody molecule comprises at least one, two, or three complementarity determining regions (CDRs) from a heavy chain variable region comprising an amino acid sequence shown in Tables 1-4, or encoded by the nucleotide sequence in Tables 1-4. In one embodiment, one or more of the CDRs (or collectively all of the CDRs) have one, two, three, four, five or more changes, e.g., amino acid substitutions, insertions, or deletions, relative to the amino acid sequence shown in Tables 1-4, or encoded by a nucleotide sequence shown in Tables 1-4. In certain embodiments, the anti-TIM-3 antibody molecule includes a substitution in a heavy chain CDR, e.g., one or more substitutions in a CDR1, CDR2 and/or CDR3 of the heavy chain. In one embodiment, the anti-TIM-3 antibody molecule includes a substitution in the heavy chain CDR2 at position 55 of the heavy chain region, e.g., a substitution of an asparagine to serine, or an asparagine to glutamine, at position 55 of the heavy chain region according to Tables 1-4 (e.g., any of SEQ ID NOs:1 or 18 for murine or humanized, unmodified; or any of SEQ ID NOs: 26, or 32 for a modified sequence).

In some embodiments, the anti-TIM-3 antibody molecule includes at least one, two, or three complementarity determining regions (CDRs) from a light chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4, or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In certain embodiments, the anti-TIM-3 antibody molecule includes at least one, two, or three CDRs (or collectively all of the CDRs) from a light chain variable region comprising an amino acid sequence shown in Tables 1-4, or encoded by a nucleotide sequence shown in Tables 1-4. In some embodiments, one or more of the CDRs (or collectively all of the CDRs) have one, two, three, four, five, six or more changes, e.g., amino acid substitutions, insertions, or deletions, relative to the CDRs shown in Tables 1-4, or encoded by a nucleotide sequence shown in Tables 1-4. In some embodiments, the anti-TIM-3 antibody molecule includes at least one, two, or three CDRs (or collectively all of the CDRs) from a light chain variable region comprising an amino acid sequence shown in Tables 1-4, or encoded by a nucleotide sequence shown in Tables 1-4. In some embodiments, one or more of the CDRs (or collectively all of the CDRs) have one, two, three, four, five, six or more changes, e.g., amino acid substitutions, insertions, or deletions, relative to the CDRs shown in Tables 1-4, or encoded by a nucleotide sequence shown in Tables 1-4.

In some embodiments, the anti-TIM-3 antibody molecule includes at least one, two, three, four, five or six CDRs (or collectively all of the CDRs) from a heavy and light chain variable region comprising an amino acid sequence shown in Tables 1-4, or encoded by a nucleotide sequence shown in Tables 1-4. In some embodiments, one or more of the CDRs (or collectively all of the CDRs) have one, two, three, four, five, six or more changes, e.g., amino acid substitutions, insertions, or deletions, relative to the CDRs shown in Tables 1-4, or encoded by a nucleotide sequence shown in Tables 1-4.

In certain embodiments, the anti-TIM-3 antibody molecule includes all six CDRs from an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4, or closely related CDRs, e.g., CDRs which are identical or which have at least one amino acid alteration, but not more than two, three or four alterations (e.g., substitutions, deletions, or insertions, e.g., conservative substitutions). In certain embodiments, the anti-TIM-3 antibody molecule may include any CDR described herein. In certain embodiments, the anti-TIM-3 antibody molecule includes a substitution in a heavy chain CDR, e.g., one or more substitutions in a CDR1, CDR2 and/or CDR3 of the heavy chain. In one embodiment, the anti-TIM-3 antibody molecule includes a substitution in the heavy chain CDR2 at position 55 of the heavy chain region, e.g., a substitution of an asparagine to serine, or an asparagine to glutamine, at position 55 of the heavy chain region according to Tables 1-4 (e.g., any of SEQ ID NOs:1 or 18 for murine or humanized, unmodified; or any of SEQ ID NOs: 26, or 32 for a modified sequence).

In some embodiments, the anti-TIM-3 antibody molecule includes at least one, two, or three CDRs according to Kabat et al. (e.g., at least one, two, or three CDRs according to the Kabat definition as set out in Tables 1-4) from a heavy chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Tables 1-4.

In certain embodiments, the anti-TIM-3 antibody molecule includes at least one, two, or three CDRs according to Kabat et al. (e.g., at least one, two, or three CDRs according to the Kabat definition as set out in Tables 1-4) from a light chain variable region of an antibody described herein, e.g., an antibody chosen from any of ABTIM, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Tables 1-4.

In certain embodiments, the anti-TIM-3 antibody molecule includes at least one, two, three, four, five, or six CDRs according to Kabat et al. (e.g., at least one, two, three, four, five, or six CDRs according to the Kabat definition as set out in Tables 1-4) from the heavy and light chain variable regions of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Tables 1-4.

In some embodiments, the anti-TIM-3 antibody molecule includes all six CDRs according to Kabat et al. (e.g., all six CDRs according to the Kabat definition as set out in Tables 1-4) from the heavy and light chain variable regions of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Tables 1-4. In one embodiment, the anti-TIM-3 antibody molecule may include any CDR described herein.

In some embodiments, the anti-TIM-3 antibody molecule includes at least one, two, or three Chothia hypervariable loops (e.g., at least one, two, or three hypervariable loops according to the Chothia definition as set out in Tables 1-4) from a heavy chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or at least the amino acids from those hypervariable loops that contact TIM-3; or which have at least one amino acid alteration, but not more than two, three or four alterations (e.g., substitutions, deletions, or insertions, e.g., conservative substitutions) relative to one, two, or three hypervariable loops according to Chothia et al. shown in Tables 1-4.

In certain embodiments, the anti-TIM-3 antibody molecule includes at least one, two, or three Chothia hypervariable loops (e.g., at least one, two, or three hypervariable loops according to the Chothia definition as set out in Tables 1-4) of a light chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or at least the amino acids from those hypervariable loops that contact TIM-3; or which have at least one amino acid alteration, but not more than two, three or four alterations (e.g., substitutions, deletions, or insertions, e.g., conservative substitutions) relative to one, two, or three hypervariable loops according to Chothia et al. shown in Tables 1-4.

In certain embodiments, the anti-TIM-3 antibody molecule includes at least one, two, three, four, five, or six hypervariable loops (e.g., at least one, two, three, four, five, or six hypervariable loops according to the Chothia definition as set out in Tables 1-4) from the heavy and light chain variable regions of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or at least the amino acids from those hypervariable loops that contact TIM-3; or which have at least one amino acid alteration, but not more than two, three or four alterations (e.g., substitutions, deletions, or insertions, e.g., conservative substitutions) relative to one, two, three, four, five or six hypervariable loops according to Chothia et al. shown in Tables 1-4.

In some embodiments, the anti-TIM-3 antibody molecule includes all six hypervariable loops (e.g., all six hypervariable loops according to the Chothia definition as set out in Tables 1-4) of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or closely related hypervariable loops, e.g., hypervariable loops which are identical or which have at least one amino acid alteration, but not more than two, three or four alterations (e.g., substitutions, deletions, or insertions, e.g., conservative substitutions); or which have at least one amino acid alteration, but not more than two, three or four alterations (e.g., substitutions, deletions, or insertions, e.g., conservative substitutions) relative to all six hypervariable loops according to Chothia et al. shown in Tables 1-4. In one embodiment, the anti-TIM-3 antibody molecule may include any hypervariable loop described herein.

In still another embodiment, the anti-TIM-3 antibody molecule includes at least one, two, or three hypervariable loops that have the same canonical structures as the corresponding hypervariable loop of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In certain embodiments, the anti-TIM-3 antibody molecule includes a combination of CDRs or hypervariable loops defined according to the Kabat et al. and Chothia et al.

In one embodiment, the anti-TIM-3 antibody molecule includes at least one, two or three CDRs or hypervariable loops from a heavy chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23, according to the Kabat and Chothia definition (e.g., at least one, two, or three CDRs or hypervariable loops according to the Kabat and Chothia definition as set out in Tables 1-4); or encoded by the nucleotide sequence in Tables 1-4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs or hypervariable loops according to Kabat and/or Chothia shown in Tables 1-4.

In another embodiment, the anti-TIM-3 antibody molecule includes at least one, two or three CDRs or hypervariable loops from a light chain variable region of an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23, according to the Kabat and Chothia definition (e.g., at least one, two, or three CDRs or hypervariable loops according to the Kabat and Chothia definition as set out in Tables 1-4); or encoded by the nucleotide sequence in Tables 1-4; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs or hypervariable loops according to Kabat and/or Chothia shown in Tables 1-4.

The anti-TIM-3 antibody molecule can contain any combination of CDRs or hypervariable loops according to the Kabat and Chothia definitions.

In some embodiments, the anti-TIM-3 antibody molecule includes at least one, two, or three Chothia hypervariable loops from a heavy chain variable region of an antibody described herein, e.g., an antibody of Tables 1-4, or at least the amino acids from those hypervariable loops that contact TIM-3.

In some embodiments, the anti-TIM-3 antibody molecule includes at least one, two, or three Chothia hypervariable loops from a light chain variable region of an antibody described herein, e.g., an antibody of Tables 1-4, or at least the amino acids from those hypervariable loops that contact TIM-3.

In some embodiments, the anti-TIM-3 antibody molecule includes at least one, two, or three Kabat hypervariable loops from a heavy chain variable region of an antibody described herein, e.g., an antibody of Tables 1-4, or at least the amino acids from those hypervariable loops that contact TIM-3.

In some embodiments, the anti-TIM-3 antibody molecule includes at least one, two, or three Kabat hypervariable loops from a light chain variable region of an antibody described herein, e.g., an antibody of Tables 1-4, or at least the amino acids from those hypervariable loops that contact TIM-3.

In certain embodiments, the anti-TIM-3 antibody molecule includes at least one, two, three, four, five, or six hypervariable loops from the heavy and light chain variable regions of an antibody described herein, e.g., an antibody of Tables 1-4, or at least the amino acids from those hypervariable loops that contact TIM-3.

In certain embodiments, the anti-TIM-3 antibody molecule includes all six hypervariable loops from the heavy and light chain variable regions of an antibody described herein, e.g., an antibody of Tables 1-4, or at least the amino acids from those hypervariable loops that contact TIM-3, or at least the amino acids from those hypervariable loops that contact TIM-3, or closely related hypervariable loops, e.g., hypervariable loops which are identical or which have at least one amino acid alteration, but not more than two, three or four alterations (e.g., substitutions, e.g., conservative substitutions, deletions, or insertions).

In some embodiments, the anti-TIM-3 antibody molecule includes at least one, two, or three hypervariable loops that have the same canonical structures as the corresponding hypervariable loop of an antibody described herein, e.g., an antibody of Tables 1-4, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references. In an embodiment, e.g., an embodiment comprising a variable region, CDR (e.g., Chothia CDR or Kabat CDR), or other sequence referred to herein, e.g., in Tables 1-4, the antibody molecule is a monospecific antibody molecule, a bispecific antibody molecule, or is an antibody molecule that comprises an antigen binding fragment of an antibody, e.g., a half antibody or antigen binding fragment of a half antibody. In certain embodiments the antibody molecule is a bispecific antibody molecule having a first binding specificity for TIM-3 and a second binding specificity for PD-1, LAG-3, CEACAM (e.g., CEACAM-1, CEACAM-3 and/or CEACAM-5), PD-L1 or PD-L2.

In certain embodiments, the light or the heavy chain variable framework (e.g., the region encompassing at least FR1, FR2, FR3, or FR4) of the anti-TIM-3 antibody molecule can be chosen from: (a) a light or heavy chain variable framework including at least 80%, 85%, 87% 90%, 92%, 93%, 95%, 97%, 98%, or preferably 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In some embodiments, the light or heavy chain variable framework region includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In certain embodiments, the anti-TIM-3 antibody molecule comprises a heavy chain variable domain having at least one, two, three, four, five, six, seven, ten, fifteen, twenty or more changes, e.g., amino acid substitutions, insertions, or deletions, from an amino acid sequence of e.g., the amino acid sequence of the FR region in the entire variable region, e.g., shown in FIG. 1A. In some embodiments, the anti-TIM-3 antibody molecule comprises a heavy chain variable domain having one or more (e.g., all) of: A at position 2, Y at position 3, S at position 7, R at position 13, V at position 37, R at position 42, V at position 72, A at position 79, or F at position 95, e.g., the amino acid sequence of the FR in the entire variable region, e.g., as shown in FIG. 1A. In some embodiments, the anti-TIM-3 antibody molecule comprises a heavy chain variable domain having 2, 3, 4, 5, 6, 7, 8, or 9 positions selected from: A at position 2, Y at position 3, S at position 7, R at position 13, V at position 37, R at position 42, V at position 72, A at position 79, or F at position 95 of the amino acid sequence of an antibody of Tables 1-4, e.g.,

In certain embodiments (and optionally in combination with the heavy chain substitutions described herein, e.g., in the previous paragraph), the anti-TM-3 antibody molecule comprises a light chain variable domain having at least one, two, three, four, five, six, seven, ten, fifteen, twenty or more amino acid changes, e.g., amino acid substitutions, insertions, or deletions, from an amino acid sequence of Tables 1-4, e.g., the amino acid sequence of the FR region in the entire variable region, e.g., shown in FIG. 1B. In certain embodiments, the anti-TIM-3 antibody comprises a light chain variable domain having M at position 89 of the amino acid sequence of an antibody of Tables 1-4.

In some embodiments, the heavy or light chain variable domain, or both, of the of the anti-TIM-3 antibody molecule includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody chosen from any of ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4; or encoded by the nucleotide sequence in Tables 1-4; or which differs at least 1 or 5 residues, but less than 40, 30, 20, or 10 residues, from a variable region of an antibody described herein.

In certain embodiments, the heavy or light chain variable region, or both, of the anti-TIM-3 antibody molecule includes an amino acid sequence encoded by a nucleic acid sequence described herein or a nucleic acid that hybridizes to a nucleic acid sequence described herein (e.g., a nucleic acid sequence as shown in Tables 1-4) or its complement, e.g., under low stringency, medium stringency, or high stringency, or other hybridization condition described herein.

In certain embodiments, the anti-TIM-3 antibody molecule comprises at least one, two, three, or four antigen-binding regions, e.g., variable regions, having an amino acid sequence as set forth in Tables 1-4, or a sequence substantially identical thereto (e.g., a sequence at least about 85%, 90%, 95%, 99% or more identical thereto, or which differs by no more than 1, 2, 5, 10, or 15 amino acid residues from the sequences shown in Tables 1-4. In certain embodiments, the anti-TIM-3 antibody molecule includes a VH and/or VL domain encoded by a nucleic acid having a nucleotide sequence that encodes an antibody of Tables 1-4, or a sequence substantially identical to any one of the nucleotide sequences (e.g., a sequence at least about 85%, 90%, 95%, 99% or more identical thereto, or which differs by no more than 3, 6, 15, 30, or 45 nucleotides from the sequences shown in Tables 1-4).

In certain embodiments, the anti-TIM-3 antibody molecule comprises at least one, two, or three (e.g., all) CDRs from a heavy chain variable region having an amino acid sequence as set forth in Tables 1-4, or a sequence substantially homologous thereto (e.g., a sequence at least about 85%, 90%, 95%, 99% or more identical thereto, and/or having one, two, three or more substitutions, insertions or deletions, e.g., conserved substitutions). In some embodiments, the anti-TIM-3 antibody molecule comprises at least one, two, or three (e.g., all) CDRs from a light chain variable region having an amino acid sequence as set forth in Tables 1-4, or a sequence substantially homologous thereto (e.g., a sequence at least about 85%, 90%, 95%, 99% or more identical thereto, and/or having one, two, three or more substitutions, insertions or deletions, e.g., conserved substitutions). In certain embodiments, the anti-TIM-3 antibody molecule comprises at least one, two, three, four, five or six (e.g., all) CDRs from heavy and light chain variable regions having an amino acid sequence as set forth in Tables 1-4, or a sequence substantially homologous thereto (e.g., a sequence at least about 85%, 90%, 95%, 99% or more identical thereto, and/or having one, two, three or more substitutions, insertions or deletions, e.g., conserved substitutions).

In some embodiments, the anti-TIM-3 antibody molecule comprises at least one, two, or three (e.g., all) CDRs and/or hypervariable loops from a heavy chain variable region having an amino acid sequence of an antibody described herein, e.g., an antibody chosen from any of ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23, as summarized in Tables 1-4, or a sequence substantially identical thereto (e.g., a sequence at least about 85%, 90%, 95%, 99% or more identical thereto, and/or having one, two, three or more substitutions, insertions or deletions, e.g., conserved substitutions). In certain embodiments, the anti-TIM-3 antibody molecule comprises at least one, two, or three (e.g., all) CDRs and/or hypervariable loops from a light chain variable region having an amino acid sequence of an antibody described herein, e.g., an antibody chosen from any of ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23, as summarized in Tables 1-4, or a sequence substantially identical thereto (e.g., a sequence at least about 85%, 90%, 95%, 99% or more identical thereto, and/or having one, two, three or more substitutions, insertions or deletions, e.g., conserved substitutions). In some embodiments, the anti-TIM-3 antibody molecule comprises all six CDRs and/or hypervariable loops described herein, e.g., described in Tables 1-4.

In some embodiments, the antibody molecule has a variable region that is identical in sequence, or which differs by 1, 2, 3, or 4 amino acids from a variable region described herein (e.g., an FR region disclosed herein).

In some embodiments, the anti-TIM-3 antibody molecule is a full antibody or fragment thereof (e.g., a Fab, F(ab′)2, Fv, or a single chain Fv fragment (scFv)). In certain embodiments, the anti-TIM-3 antibody molecule is a monoclonal antibody or an antibody with single specificity. The anti-TIM-3 antibody molecule can also be a humanized, chimeric, camelid, shark, or in vitro-generated antibody molecule. In some embodiments, the anti-TIM-3 antibody molecule thereof is a humanized antibody molecule. The heavy and light chains of the anti-TIM-3 antibody molecule can be full-length (e.g., an antibody can include at least one or at least two complete heavy chains, and at least one or at least two complete light chains) or can include an antigen-binding fragment (e.g., a Fab, F(ab′)2, Fv, a single chain Fv fragment, a single domain antibody, a diabody (dAb), a bivalent or bispecific antibody or fragment thereof, a single domain variant thereof, or a camelid antibody).

In certain embodiments, the anti-TIM-3 antibody molecule is in the form of a bispecific or multispecific antibody molecule. In one embodiment, the bispecific antibody molecule has a first binding specificity to TIM-3 and a second binding specifity, e.g., a second binding specificity to PD-1, LAG-3, CEACAM (e.g., CEACAM-1, -3 and/or -5), PD-L1 or PD-L2. In one embodiment, the bispecific antibody molecule binds to TIM-3 and PD-1. In another embodiment, the bispecific antibody molecule binds to TIM-3 and LAG-3. In another embodiment, the bispecific antibody molecule binds to TIM-3 and CEACAM (e.g., CEACAM-1, -3 and/or -5). In another embodiment, the bispecific antibody molecule binds to TIM-3 and CEACAM-1. In another embodiment, the bispecific antibody molecule binds to TIM-3 and CEACAM-3. In yet another embodiment, the bispecific antibody molecule binds to TIM-3 and CEACAM-5. In another embodiment, the bispecific antibody molecule binds to TIM-3 and PD-L1. In yet another embodiment, the bispecific antibody molecule binds to TIM-3 and PD-L2. Any combination of the aforesaid molecules can be made in a multispecific antibody molecule, e.g., a trispecific antibody that includes a first binding specificity to TIM-3, and a second and third binding specifities to one or more of: PD-1, LAG-3, CEACAM (e.g., CEACAM-1, -3 and/or -5), PD-L1 or PD-L2.

In other embodiments, the anti-TIM-3 antibody molecule is used in combination with a bispecific molecule comprising one or more of: PD-1, LAG-3, CEACAM (e.g., CEACAM-1, -3 and/or -5), PD-L1 or PD-L2. In one embodiment, the bispecific antibody molecule used in combination binds to CEACAM (e.g., CEACAM-1, -3 and/or -5) and LAG-3. In another embodiment, the bispecific antibody molecule used in combination binds to CEACAM (e.g., CEACAM-1, -3 and/or -5) and PD-1. In another embodiment, the bispecific antibody molecule used in combination binds to LAG-3 and PD-1.

In certain embodiments, the anti-TIM-3 antibody molecule has a heavy chain constant region (Fc) chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant region of IgG1 or IgG2 (e.g., human IgG1 or IgG2). In some embodiments, the heavy chain constant region is human IgG1. In some embodiments, the anti-TIM-3 antibody molecule has a light chain constant region chosen from, e.g., the light chain constant regions of kappa or lambda, in some embodiments kappa (e.g., human kappa). In some embodiments, the constant region is altered, e.g., mutated, to modify the properties of the anti-TIM-3 antibody molecule (e.g., to increase or decrease one or more of: Fc receptor binding, antibody glycosylation, the number of cysteine residues, effector cell function, or complement function). For example, the constant region may be mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 108 or 110; or positions 135 (M to Y), 137 (S to T), 139 (T to E), 316 (H to K) and 317 (N to F) of SEQ ID NOs: 111, 112, 113 or 114). In another embodiment, the heavy chain constant region of an IgG4, e.g., a human IgG4, is mutated at position 228 according to EU numbering (e.g., S to P), e.g., as shown in Table 5. In certain embodiments, the anti-TIM-3 antibody molecules comprises a human IgG4 mutated at position 228 according to EU numbering (e.g., S to P), e.g., as shown in Table 5; and a kappa light chain constant region, e.g., as shown in Table 5. In still another embodiment, the heavy chain constant region of an IgG1, e.g., a human IgG1, is mutated at one or more of position 297 (e.g., N to A), position 265 (e.g., D to A), position 329 (e.g., P to A), position 234 (e.g., L to A), or position 235 (e.g., L to A), all according to EU numbering, e.g., as shown in Table 5. In certain embodiments, the anti-TIM-3 antibody molecules comprises a human IgG1 mutated at one or more of the aforesaid positions, e.g., as shown in Table 5; and a kappa light chain constant region, e.g., as shown in Table 5. In some embodiments, the anti-TIM-3 antibody molecule is a humanized antibody molecule.

In some embodiments, the anti-TIM-3 antibody molecules comprise combinations of human or humanized framework regions with CDRs (complementarity determining regions).

The combinations disclosed herein (e.g., the combinations comprising anti-TIM-3 antibody molecules disclosed herein) can inhibit, reduce or neutralize one or more activities of TIM-3, e.g., resulting in blockade or reduction of an immune checkpoint. In one embodiment, the antibody molecule results in one or more of: enhancing IFN-gamma and/or TNF alpha section in T cells; enhancing proliferation in T cells, e.g., CD4+ or CD8+ T cells; enhancing NK cell cytotoxic activity; or reducing suppressor activity of regulatory T cells (Tregs) or macrophages. Thus, such combinations can be used to treat or prevent disorders where enhancing an immune response in a subject is desired.

Exemplary sequences of anti-TIM-3 antibodies are described in the Tables 1-4 below.

TABLE 1
Summary of the sequences of the murine antibody ABTIM3.

| Antibody designation | SEQ ID NO | Description |
|---|---|---|
| ABTIM3 | 1 | VH amino acid sequence |
| | 2 | VL amino acid sequence |
| | 3 | VHCDR1 amino acid sequence |
| | 4 | VHCDR2 amino acid sequence |
| | 5 | VHCDR3 amino acid sequence |
| | 6 | VLCDR1 amino acid sequence |
| | 7 | VLCDR2 amino acid sequence |
| | 8 | VLCDR3 amino acid sequence |

TABLE 2
Depiction of the amino acid sequences of the
murine antibody ABTIM3 heavy chain variable
domain and light chain variable domain.

| SEQ | |
|---|---|
| ID | |
| NO | Sequence |
| 1 | 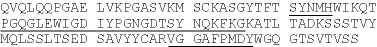 |
| 2 | 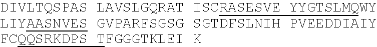 |

CDRs are underlined.

TABLE 3
Depiction of the amino acid sequences of the
murine antibody ABTIM3 heavy chain CDRs and
light chain CDRs.

| SEQ ID | |
|---|---|
| NO | Sequence |
| 3 | SYNMH |
| 4 | DIYPGNGDTSYNQKFKG |
| 5 | VGGAFPMDY |
| 6 | RASESVEYYGTSLMQ |
| 7 | AASNVES |
| 8 | QQSRKDPST |

Exemplary sequences of anti-TIM-3 antibodies are described in Table 4. The antibody molecules include murine ABTIM3, and humanized antibody molecules. The amino acid and nucleotide sequences of the heavy and light chain CDRs, the heavy and light chain variable regions, and the heavy and light chains are shown.

TABLE 4
Summary of the sequences of exemplary anti-TIM-3 antibodies.

| Hybridoma | | |
|---|---|---|
| clone | | |
| ABTIM3 | | |
| SEQ ID NO: 3 | HCDR1 | SYNMH |
| (Kabat) | | |
| SEQ ID NO: 4 | HCDR2 | DIYPGNGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 10 | HCDR2 | YPGNGD |
| (Chothia) | | |
| SEQ ID NO: 5 | HCDR3 | VGGAFPMDY |
| (Chothia) | | |
| SEQ ID NO: 1 | VH | QVQLQQPGAELVKPGASVKMSCKASGYTFTSYNMHWIKQTP |
| | | GQGLEWIGDIYPGNGDTSYNQKFKGKATLTADKSSSTVYMQL |
| | | SSLTSEDSAVYYCARVGGAFPMDYWGQGTSVTVSS |
| SEQ ID NO: 11 | DNA VH | CAGGTGCAACTGCAGCAGCCTGGGGCTGAGCTGGTGAAG |
| | | CCTGGGGCCTCAGTGAAGATGTCCTGCAAGGCTTCTGGCT |
| | | ACACATTTACCAGTTACAATATGCACTGGATAAAGCAGACA |
| | | CCTGGACAGGGCCTGGAATGGATTGGAGATATTTATCCAG |
| | | GAAATGGTGATACTTCCTACAATCAGAAATTCAAAGGCAAG |
| | | GCCACATTGACTGCAGACAAATCCTCCAGCACAGTCTACAT |
| | | GCAGCTCAGCAGCCTGACATCTGAGGACTCTGCGGTCTAT |
| | | TACTGTGCAAGAGTGGGGGGTGCCTTTCCTATGGACTACT |
| | | GGGGTCAAGGAACCTCAGTCACCGTCTCCTCA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 2 | VL | DIVLTQSPASLAVSLGQRATISCRASESVEYYGTSLMQWYQQ |
| | | KPGQPPKLLIYAASNVESGVPARFSGSGSGTDFSLNIHPVEED |
| | | DIAIYFCQQSRKDPSTFGGGTKLEIK |
| SEQ ID NO: 15 | DNA VL | GACATTGTGCTCACCCAATCTCCAGCTTCTTTGGCTGTGTC |
| | | TCTAGGGCAGAGAGCCACCATCTCCTGCAGAGCCAGTGAA |
| | | AGTGTTGAATATTATGGCACAAGTTTAATGCAGTGGTACCA |
| | | ACAGAAACCAGGACAGCCACCCAAACTCCTCATCTATGCT |
| | | GCATCCAACGTAGAATCTGGGGTCCCTGCCAGGTTTAGTG |
| | | GCAGTGGGTCTGGGACAGACTTCAGCCTCAACATCCATCC |
| | | TGTGGAGGAGGATGATATTGCAATATATTTCTGTCAGCAAA |
| | | GTAGGAAGGATCCTTCGACGTTCGGTGGAGGCACCAAGCT |
| | | GGAGATCAAA |
| ABTIM3- | | |
| hum01 | | |
| SEQ ID NO: 3 | HCDR1 | SYNMH |
| (Kabat) | | |
| SEQ ID NO: 4 | HVDR2 | DIYPGNGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 10 | HCDR2 | YPGNGD |
| (Chothia) | | |
| SEQ ID NO: 5 | HCDR3 | VGGAFPMDY |
| (Chothia) | | |
| SEQ ID NO: 16 | VH | QVQLVQSGAEVKKPGASVKVSCKASGYTFTSYNMHWVRQA |
| | | PGQGLEWIGDIYPGNGDTSYNQKFKGRATMTADKSTSTVYM |
| | | ELSSLRSEDTAVYYCARVGGAFPMDYWGQGTLVTVSS |
| SEQ ID NO: 17 | DNA VH | CAGGTGCAGCTGGTGCAGTCAGGCGCCGAAGTGAAGAAA |
| | | CCCGGCGCTAGTGTGAAAGTCTCTTGTAAAGCTAGTGGCT |
| | | ACACCTTCACTAGCTATAATATGCACTGGGTTCGCCAGGCC |
| | | CCAGGGCAGGGCCTCGAGTGGATCGGCGATATCTACCCC |
| | | GGGAACGGCGACACTAGTTATAATCAGAAGTTTAAGGGTA |
| | | GAGCTACTATGACCGCCGATAAGTCTACTAGCACCGTCTAT |
| | | ATGGAACTGAGTTCCCTGAGGTCTGAGGACACCGCCGTCT |
| | | ACTACTGCGCTAGAGTGGGCGGAGCCTTCCCTATGGACTA |
| | | CTGGGGTCAAGGCACCCTGGTCACCGTGTCTAGC |
| SEQ ID NO: 18 | Heavy | QVQLVQSGAEVKKPGASVKVSCKASGYTFTSYNMHWVRQA |
| | Chain | PGQGLEWIGDIYPGNGDTSYNQKFKGRATMTADKSTSTVYM |
| | | ELSSLRSEDTAVYYCARVGGAFPMDYWGQGTLVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPS |
| | | DIAVEWESNGQPNNYKTTPPVLDSDGSFFLYSRLTVDKSRW |
| | | QEGNVFSCSVMHEALHNHYTQKSLSLSG |
| SEQ ID NO: 19 | DNA | CAGGTGCAGCTGGTGCAGTCAGGCGCCGAAGTGAAGAAA |
| | Heavy | CCCGGCGCTAGTGTGAAAGTCTCTTGTAAAGCTAGTGGCT |
| | Chain | ACACCTTCACTAGCTATAATATGCACTGGGTTCGCCAGGCC |
| | | CCAGGGCAGGGCCTCGAGTGGATCGGCGATATCTACCCC |
| | | GGGAACGGCGACACTAGTTATAATCAGAAGTTTAAGGGTA |
| | | GAGCTACTATGACCGCCGATAAGTCTACTAGCACCGTCTAT |
| | | ATGGAACTGAGTTCCCTGAGGTCTGAGGACACCGCCGTCT |
| | | ACTACTGCGCTAGAGTGGGCGGAGCCTTCCCTATGGACTA |
| | | CTGGGGTCAAGGCACCCTGGTCACCGTGTCTAGCGCTAGC |
| | | ACTAAGGGCCCGTCCGTGTTCCCCCTGGCACCTTGTAGCC |
| | | GGAGCACTAGCGAATCCACCGCTGCCCTCGGCTGCCTGGT |
| | | CAAGGATTACTTCCCGGAGCCCGTGACCGTGTCCTGGAAC |
| | | AGCGGAGCCCTGACCTCCGGAGTGCACACCTTCCCCGCT |
| | | GTGCTGCAGAGCTCCGGGCTGTACTCGCTGTCGTCGGTG |
| | | GTCACGGTGCCTTCATCTAGCCTGGGTACCAAGACCTACA |
| | | CTTGCAACGTGGACCACAAGCCTTCCAACACTAAGGTGGA |
| | | CAAGCGCGTCGAATCGAAGTACGGCCCACCGTGCCCGCC |
| | | TTGTCCCGCGCCGGAGTTCCTCGGCGGTCCCTCGGTCTTT |
| | | CTGTTCCCACCGAAGCCCAAGGACACTTTGATGATTTCCCG |
| | | CACCCCTGAAGTGACATGCGTGGTCGTGGACGTGTCACAG |
| | | GAAGATCCGGAGGTGCAGTTCAATTGGTACGTGGATGGCG |
| | | TCGAGGTGCACAACGCCAAAACCAAGCCGAGGGAGGAGC |
| | | AGTTCAACTCCACTTACCGCGTCGTGTCCGTGCTGACGGT |
| | | GCTGCATCAGGACTGGCTGAACGGGAAGGAGTACAAGTGC |
| | | AAAGTGTCCAACAAGGGACTTCCTAGCTCAATCGAAAAGAC |
| | | CATCTCGAAAGCCAAGGGACAGCCCCGGGAACCCCAAGT |
| | | GTATACCCTGCCACCGAGCCAGGAAGAAATGACTAAGAAC |
| | | CAAGTCTCATTGACTTGCCTTGTGAAGGGCTTCTACCCATC |
| | | GGATATCGCCGTGGAATGGGAGTCCAACGGCCAGCCGGA |
| | | AAACAACTACAAGACCACCCCTCCGGTGCTGGACTCAGAC |
| | | GGATCCTTCTTCCTCTACTCGCGGCTGACCGTGGATAAGA |
| | | GCAGATGGCAGGAGGGAAATGTGTTCAGCTGTTCTGTGAT |
| | | GCATGAAGCCCTGCACAACCACTACACTCAGAAGTCCCTG |
| | | TCCCTCTCCCTGGGA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 20 | VL | DIVLTQSPDSLAVSLGERATINCRASESVEYYGTSLMQWYQQ |
| | | KPGQPPKLLIYAASNVESGVPDRFSGSGSGTDFTLTISSLQAE |
| | | DVAVYYCQQSRKDPSTFGGGTKVEIK |
| SEQ ID NO: 21 | DNA VL | GATATCGTCCTGACTCAGTCACCCGATAGCCTGGCCGTCA |
| | | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTACTGTCAGC |
| | | AGTCTAGGAAGGACCCTAGCACCTTCGGCGGAGGCACTAA |
| | | GGTCGAGATTAAG |
| SEQ ID NO: 22 | Light | DIVLTQSPDSLAVSLGERATINCRASESVEYYGTSLMQWYQQ |
| | Chain | KPGQPPKLLIYAASNVESGVPDRFSGSGSGTDFTLTISSLQAE |
| | | DVAVYYCQQSRKDPSTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 23 | DNA Light | GATATCGTCCTGACTCAGTCACCCGATAGCCTGGCCGTCA |
| | Chain | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTACTGTCAGC |
| | | AGTCTAGGAAGGACCCTAGCACCTTCGGCGGAGGCACTAA |
| | | GGTCGAGATTAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum02 | | |
| SEQ ID NO: 3 | HCDR1 | SYNMH |
| (Kabat) | | |
| SEQ ID NO: 24 | HCDR2 | DIYPGSGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 25 | HCDR2 | YPGSGD |
| (Chothia) | | |
| SEQ ID NO: 5 | HCDR3 | VGGAFPMDY |
| (Chothia) | | |
| SEQ ID NO: 26 | VH | QVQLVQSGAEVKKPGASVKVSCKASGYTFTSYNMHWVRQA |
| | | PGQGLEWIGDIYPGSGDTSYNQKFKGRATMTADKSTSTVYM |
| | | ELSSLRSEDTAVYYCARVGGAFPMDYWGQGTLVTVSS |
| SEQ ID NO: 27 | DNA VH | CAGGTGCAGCTGGTGCAGTCAGGCGCCGAAGTGAAGAAA |
| | | CCCGGCGCTAGTGTGAAAGTTAGCTGTAAAGCTAGTGGCT |
| | | ACACCTTCACTAGCTATAATATGCACTGGGTTCGCCAGGCC |
| | | CCAGGTCAAGGCCTCGAGTGGATCGGCGATATCTACCCCG |
| | | GTAGCGGCGACACTAGTTATAATCAGAAGTTTAAGGGTAGA |
| | | GCTACTATGACCGCCGATAAGTCTACTAGCACCGTCTATAT |
| | | GGAACTGAGTTCCCTGAGGTCTGAAGATACCGCCGTCTAC |
| | | TACTGCGCTAGAGTGGGCGGAGCCTTCCCTATGGACTACT |
| | | GGGGTCAAGGCACCCTGGTCACCGTGTCTAGC |
| SEQ ID NO: 28 | Heavy | QVQLVQSGAEVKKPGASVKVSCKASGYTFTSYNMHWVRQA |
| | Chain | PGQGLEWIGDIYPGSGDTSYNQKFKGRATMTADKSTSTVYM |
| | | ELSSLRSEDTAVYYCARVGGAFPMDYWGQGTLVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPS |
| | | DIAVEWESNGQPENNYKTTPPVLDSDGSFFLYSRLTVDKSRW |
| | | QEGNVFSCSVMHEALHNHYTQKSLSLSLG |
| SEQ ID NO: 29 | DNA | CAGGTGCAGCTGGTGCAGTCAGGCGCCGAAGTGAAGAAA |
| | Heavy | CCCGGCGCTAGTGTGAAAGTTAGCTGTAAAGCTAGTGGCT |
| | Chain | ACACCTTCACTAGCTATAATATGCACTGGGTTCGCCAGGCC |
| | | CCAGGTCAAGGCCTCGAGTGGATCGGCGATATCTACCCCG |
| | | GTAGCGGCGACACTAGTTATAATCAGAAGTTTAAGGGTAGA |
| | | GCTACTATGACCGCCGATAAGTCTACTAGCACCGTCTATAT |
| | | GGAACTGAGTTCCCTGAGGTCTGAAGATACCGCCGTCTAC |
| | | TACTGCGCTAGAGTGGGCGGAGCCTTCCCTATGGACTACT |
| | | GGGGTCAAGGCACCCTGGTCACCGTGTCTAGCGCTAGCAC |
| | | TAAGGGCCCGTCCGTGTTCCCCCTGGCACCTTGTAGCCGG |
| | | AGCACTAGCGAATCCACCGCTGCCCTCGGCTGCCTGGTCA |
| | | AGGATTACTTCCCGGAGCCCGTGACCGTGTCCTGGAACAG |
| | | CGGAGCCCTGACCTCCGGAGTGCACACCTTCCCCGCTGTG |
| | | CTGCAGAGCTCCGGGCTGTACTCGCTGTCGTCGGTGGTCA |
| | | CGGTGCCTTCATCTAGCCTGGGTACCAAGACCTACACTTG |
| | | CAACGTGGACCACAAGCCTTCCAACACTAAGGTGGACAAG |
| | | CGCGTCGAATCGAAGTACGGCCCACCGTGCCCGCCTTGTC |
| | | CCGCGCCGGAGTTCCTCGGCGGTCCCTCGGTCTTTCTGTT |
| | | CCCACCGAAGCCCAAGGACACTTTGATGATTTCCCGCACC |
| | | CCTGAAGTGACATGCGTGGTCGTGGACGTGTCACAGGAAG |
| | | ATCCGGAGGTGCAGTTCAATTGGTACGTGGATGGCGTCGA |
| | | GGTGCACAACGCCAAAACCAAGCCGAGGGAGGAGCAGTT |
| | | CAACTCCACTTACCGCGTCGTGTCCGTGCTGACGGTGCTG |
| | | CATCAGGACTGGCTGAACGGGAAGGAGTACAAGTGCAAAG |
| | | TGTCCAACAAGGGACTTCCTAGCTCAATCGAAAAGACCATC |
| | | TCGAAAGCCAAGGGACAGCCCCGGGAACCCCAAGTGTATA |
| | | CCCTGCCACCGAGCCAGGAAGAAATGACTAAGAACCAAGT |
| | | CTCATTGACTTGCCTTGTGAAGGGCTTCTACCCATCGGATA |
| | | TCGCCGTGGAATGGGAGTCCAACGGCCAGCCGGAAAACA |
| | | ACTACAAGACCACCCCTCCGGTGCTGGACTCAGACGGATC |
| | | CTTCTTCCTCTACTCGCGGCTGACCGTGGATAAGAGCAGA |
| | | TGGCAGGAGGGAAATGTGTTCAGCTGTTCTGTGATGCATG |
| | | AAGCCCTGCACAACCACTACACTCAGAAGTCCCTGTCCCT |
| | | CTCCCTGGGA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 20 | VL | DIVLTQSPDSLAVSLGERATINCRASESVEYYGTSLMQWYQQ |
| | | KPGQPPKLLIYAASNVESGVPDRFSGSGSGTDFTLTISSLQAE |
| | | DVAVYYCQQSRKDPSTFGGGTKVEIK |
| SEQ ID NO: 21 | DNA VL | GATATCGTCCTGACTCAGTCACCCGATAGCCTGGCCGTCA |
| | | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTACTGTCAGC |
| | | AGTCTAGGAAGGACCCTAGCACCTTCGGCGGAGGCACTAA |
| | | GGTCGAGATTAAG |
| SEQ ID NO: 22 | Light | DIVLTQSPDSLAVSLGERATINCRASESVEYYGTSLMQWYQQ |
| | Chain | KPGQPPKLLIYAASNVESGVPDRFSGSGSGTDFTLTISSLQAE |
| | | DVAVYYCQQSRKDPSTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 23 | DNA Light | GATATCGTCCTGACTCAGTCACCCGATAGCCTGGCCGTCA |
| | Chain | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTACTGTCAGC |
| | | AGTCTAGGAAGGACCCTAGCACCTTCGGCGGAGGCACTAA |
| | | GGTCGAGATTAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum03 | | |
| SEQ ID NO: 3 | HCDR1 | SYNMH |
| (Kabat) | | |
| SEQ ID NO: 30 | HCDR2 | DIYPGQGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 31 | HCDR2 | YPGQGD |
| (Chothia) | | |
| SEQ ID NO: 5 | HCDR3 | VGGAFPMDY |
| (Chothia) | | |
| SEQ ID NO: 32 | VH | QVQLVQSGAEVKKPGASVKVSCKASGYTFTSYNMHWVRQA |
| | | PGQGLEWIGDIYPGQGDTSYNQKFKGRATMTADKSTSTVYM |
| | | ELSSLRSEDTAVYYCARVGGAFPMDYWGQGTLVTVSS |
| SEQ ID NO: 33 | DNA VH | CAGGTGCAGCTGGTGCAGTCAGGCGCCGAAGTGAAGAAA |
| | | CCCGGCGCTAGTGTGAAAGTTAGCTGTAAAGCTAGTGGCT |
| | | ATACTTTCACTTCTTATAATATGCACTGGGTCCGCCAGGCC |
| | | CCAGGTCAAGGCCTCGAGTGGATCGGCGATATCTACCCCG |
| | | GTCAAGGCGACACTTCCTATAATCAGAAGTTTAAGGGTAGA |
| | | GCTACTATGACCGCCGATAAGTCTACTTCTACCGTCTATAT |
| | | GGAACTGAGTTCCCTGAGGTCTGAGGACACCGCCGTCTAC |
| | | TACTGCGCTAGAGTGGGCGGAGCCTTCCCAATGGACTACT |
| | | GGGGTCAAGGCACCCTGGTCACCGTGTCTAGC |
| SEQ ID NO: 34 | Heavy | QVQLVQSGAEVKKPGASVKVSCKASGYTFTSYNMHWVRQA |
| | Chain | PGQGLEWIGDIYPGQGDTSYNQKFKGRATMTADKSTSTVYM |
| | | ELSSLRSEDTAVYYCARVGGAFPMDYWGQGTLVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPS |
| | | DIAVEWESNGQPENNYKTTPPVLDSDGSFFLYSRLTVDKSRW |
| | | QEGNVFSCSVMHEALHNHYTQKSLSLSLG |
| SEQ ID NO: 35 | DNA | CAGGTGCAGCTGGTGCAGTCAGGCGCCGAAGTGAAGAAA |
| | Heavy | CCCGGCGCTAGTGTGAAAGTTAGCTGTAAAGCTAGTGGCT |
| | Chain | ATACTTTCACTTCTTATAATATGCACTGGGTCCGCCAGGCC |
| | | CCAGGTCAAGGCCTCGAGTGGATCGGCGATATCTACCCCG |
| | | GTCAAGGCGACACTTCCTATAATCAGAAGTTTAAGGGTAGA |
| | | GCTACTATGACCGCCGATAAGTCTACTTCTACCGTCTATAT |
| | | GGAACTGAGTTCCCTGAGGTCTGAGGACACCGCCGTCTAC |
| | | TACTGCGCTAGAGTGGGCGGAGCCTTCCCAATGGACTACT |
| | | GGGGTCAAGGCACCCTGGTCACCGTGTCTAGCGCTAGCAC |
| | | TAAGGGCCCGTCCGTGTTCCCCCTGGCACCTTGTAGCCGG |
| | | AGCACTAGCGAATCCACCGCTGCCCTCGGCTGCCTGGTCA |
| | | AGGATTACTTCCCGGAGCCCGTGACCGTGTCCTGGAACAG |
| | | CGGAGCCCTGACCTCCGGAGTGCACACCTTCCCCGCTGTG |
| | | CTGCAGAGCTCCGGGCTGTACTCGCTGTCGTCGGTGGTCA |
| | | CGGTGCCTTCATCTAGCCTGGGTACCAAGACCTACACTTG |
| | | CAACGTGGACCACAAGCCTTCCAACACTAAGGTGGACAAG |
| | | CGCGTCGAATCGAAGTACGGCCCACCGTGCCCGCCTTGTC |
| | | CCGCGCCGGAGTTCCTCGGCGGTCCCTCGGTCTTTCTGTT |
| | | CCCACCGAAGCCCAAGGACACTTTGATGATTTCCCGCACC |
| | | CCTGAAGTGACATGCGTGGTCGTGGACGTGTCACAGGAAG |
| | | ATCCGGAGGTGCAGTTCAATTGGTACGTGGATGGCGTCGA |
| | | GGTGCACAACGCCAAAACCAAGCCGAGGGAGGAGCAGTT |
| | | CAACTCCACTTACCGCGTCGTGTCCGTGCTGACGGTGCTG |
| | | CATCAGGACTGGCTGAACGGGAAGGAGTACAAGTGCAAAG |
| | | TGTCCAACAAGGGACTTCCTAGCTCAATCGAAAAGACCATC |
| | | TCGAAAGCCAAGGGACAGCCCCGGGAACCCCAAGTGTATA |
| | | CCCTGCCACCGAGCCAGGAAGAAATGACTAAGAACCAAGT |
| | | CTCATTGACTTGCCTTGTGAAGGGCTTCTACCCATCGGATA |
| | | TCGCCGTGGAATGGGAGTCCAACGGCCAGCCGGAAAACA |
| | | ACTACAAGACCACCCCTCCGGTGCTGGACTCAGACGGATC |
| | | CTTCTTCCTCTACTCGCGGCTGACCGTGGATAAGAGCAGA |
| | | TGGCAGGAGGGAAATGTGTTCAGCTGTTCTGTGATGCATG |
| | | AAGCCCTGCACAACCACTACACTCAGAAGTCCCTGTCCCT |
| | | CTCCCTGGGA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 20 | VL | DIVLTQSPDSLAVSLGERATINCRASESVEYYGTSLMQWYQQ |
| | | KPGQPPKLLIYAASNVESGVPDRFSGSGSGTDFTLTISSLQAE |
| | | DVAVYYCQQSRKDPSTFGGGTKVEIK |
| SEQ ID NO: 21 | DNA VL | GATATCGTCCTGACTCAGTCACCCGATAGCCTGGCCGTCA |
| | | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTACTGTCAGC |
| | | AGTCTAGGAAGGACCCTAGCACCTTCGGCGGAGGCACTAA |
| | | GGTCGAGATTAAG |
| SEQ ID NO: 22 | Light | DIVLTQSPDSLAVSLGERATINCRASESVEYYGTSLMQWYQQ |
| | Chain | KPGQPPKLLIYAASNVESGVPDRFSGSGSGTDFTLTISSLQAE |
| | | DVAVYYCQQSRKDPSTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 23 | DNA Light | GATATCGTCCTGACTCAGTCACCCGATAGCCTGGCCGTCA |
| | Chain | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTACTGTCAGC |
| | | AGTCTAGGAAGGACCCTAGCACCTTCGGCGGAGGCACTAA |
| | | GGTCGAGATTAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum04 | | |
| SEQ ID NO: 3 | HCDR1 | SYNMH |
| (Kabat) | | |
| SEQ ID NO: 4 | HCDR2 | DIYPGNGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 10 | HCDR2 | YPGNGD |
| (Chothia) | | |
| SEQ ID NO: 5 | HCDR3 | VGGAFPMDY |
| (Chothia) | | |
| SEQ ID NO: 36 | VH | QVQLVQSGAEVKKPGASVKVSCKASGYTFTSYNMHWIRQAP |
| | | GQGLEWIGDIYPGNGDTSYNQKFKGRATLTADKSTSTVYMEL |
| | | SSLRSEDTAVYYCARVGGAFPMDYWGQGTLVTVSS |
| SEQ ID NO: 37 | DNA VH | CAGGTGCAGCTGGTGCAGTCAGGCGCCGAAGTGAAGAAA |
| | | CCCGGCGCTAGTGTGAAAGTTTCTTGTAAAGCTAGTGGCTA |
| | | CACCTTCACTAGCTATAATATGCACTGGATTAGACAGGCCC |
| | | CAGGGCAGGGCCTCGAGTGGATCGGCGATATCTACCCCG |
| | | GGAACGGCGACACTAGTTATAATCAGAAGTTTAAGGGTAGA |
| | | GCTACCCTGACCGCCGATAAGTCTACTAGCACCGTCTATAT |
| | | GGAACTGAGTTCCCTGAGGTCTGAGGACACCGCCGTCTAC |
| | | TACTGCGCTAGAGTGGGCGGAGCCTTCCCTATGGACTACT |
| | | GGGGGCAGGGCACCCTGGTCACCGTGTCTAGC |
| SEQ ID NO: 38 | Heavy | QVQLVQSGAEVKKPGASVKVSCKASGYTFTSYNMHWIRQAP |
| | Chain | GQGLEWIGDIYPGNGDTSYNQKFKGRATLTADKSTSTVYMEL |
| | | SSLRSEDTAVYYCARVGGAFPMDYWGQGTLVTVSSASTKGP |
| | | SVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQE |
| | | GNVFSCSVMHEALHNHYTQKSLSLSLG |
| SEQ ID NO: 39 | DNA | CAGGTGCAGCTGGTGCAGTCAGGCGCCGAAGTGAAGAAA |
| | Heavy | CCCGGCGCTAGTGTGAAAGTTTCTTGTAAAGCTAGTGGCTA |
| | Chain | CACCTTCACTAGCTATAATATGCACTGGATTAGACAGGCCC |
| | | CAGGGCAGGGCCTCGAGTGGATCGGCGATATCTACCCCG |
| | | GGAACGGCGACACTAGTTATAATCAGAAGTTTAAGGGTAGA |
| | | GCTACCCTGACCGCCGATAAGTCTACTAGCACCGTCTATAT |
| | | GGAACTGAGTTCCCTGAGGTCTGAGGACACCGCCGTCTAC |
| | | TACTGCGCTAGAGTGGGCGGAGCCTTCCCTATGGACTACT |
| | | GGGGGCAGGGCACCCTGGTCACCGTGTCTAGCGCTAGCA |
| | | CTAAGGGCCCGTCCGTGTTCCCCCTGGCACCTTGTAGCCG |
| | | GAGCACTAGCGAATCCACCGCTGCCCTCGGCTGCCTGGTC |
| | | AAGGATTACTTCCCGGAGCCCGTGACCGTGTCCTGGAACA |
| | | GCGGAGCCCTGACCTCCGGAGTGCACACCTTCCCCGCTGT |
| | | GCTGCAGAGCTCCGGGCTGTACTCGCTGTCGTCGGTGGTC |
| | | ACGGTGCCTTCATCTAGCCTGGGTACCAAGACCTACACTT |
| | | GCAACGTGGACCACAAGCCTTCCAACACTAAGGTGGACAA |
| | | GCGCGTCGAATCGAAGTACGGCCCACCGTGCCCGCCTTGT |
| | | CCCGCGCCGGAGTTCCTCGGCGGTCCCTCGGTCTTTCTGT |
| | | TCCCACCGAAGCCCAAGGACACTTTGATGATTTCCCGCAC |
| | | CCCTGAAGTGACATGCGTGGTCGTGGACGTGTCACAGGAA |
| | | GATCCGGAGGTGCAGTTCAATTGGTACGTGGATGGCGTCG |
| | | AGGTGCACAACGCCAAAACCAAGCCGAGGGAGGAGCAGT |
| | | TCAACTCCACTTACCGCGTCGTGTCCGTGCTGACGGTGCT |
| | | GCATCAGGACTGGCTGAACGGGAAGGAGTACAAGTGCAAA |
| | | GTGTCCAACAAGGGACTTCCTAGCTCAATCGAAAAGACCAT |
| | | CTCGAAAGCCAAGGGACAGCCCCGGGAACCCCAAGTGTAT |
| | | ACCCTGCCACCGAGCCAGGAAGAAATGACTAAGAACCAAG |
| | | TCTCATTGACTTGCCTTGTGAAGGGCTTCTACCCATCGGAT |
| | | ATCGCCGTGGAATGGGAGTCCAACGGCCAGCCGGAAAAC |
| | | AACTACAAGACCACCCCTCCGGTGCTGGACTCAGACGGAT |
| | | CCTTCTTCCTCTACTCGCGGCTGACCGTGGATAAGAGCAG |
| | | ATGGCAGGAGGGAAATGTGTTCAGCTGTTCTGTGATGCAT |
| | | GAAGCCCTGCACAACCACTACACTCAGAAGTCCCTGTCCC |
| | | TCTCCCTGGGA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 40 | VL | DIVLTQSPDSLAVSLGERATINCRASESVYYGTSLMQWYQQ |
| | | KPGQPPKLLIYAASNVESGVPDRFSGSGSGTDFTLTISSLQAE |
| | | DVAVYFCQQSRKDPSTFGGGTKVEIK |
| SEQ ID NO: 41 | DNA VL | GATATCGTCCTGACTCAGTCACCCGATAGCCTGGCCGTCA |
| | | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTTCTGTCAGC |
| | | AGTCTAGGAAGGACCCTAGCACCTTCGGCGGAGGCACTAA |
| | | GGTCGAGATTAAG |
| SEQ ID NO: 42 | Light | DIVLTQSPDSLAVSLGERATINCRASESVEYYGTSLMQWYQQ |
| | Chain | KPGQPPKLLIYAASNVESGVPDRFSGSGSGTDFTLTISSLQAE |
| | | DVAVYFCQQSRKDPSTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 43 | DNA Light | GATATCGTCCTGACTCAGTCACCCGATAGCCTGGCCGTCA |
| | Chain | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTTCTGTCAGC |
| | | GGTCGAGATTAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum05 | | |
| SEQ ID NO: 3 | HCDR1 | SYNMH |
| (Kabat) | | |
| SEQ ID NO: 24 | HCDR2 | DIYPGSGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 25 | HCDR2 | YPGSGD |
| (Chothia) | | |
| SEQ ID NO: 5 | HCDR3 | VGGAFPMDY |
| (Chothia) | | |
| SEQ ID NO: 44 | VH | QVQLVQSGAEVKKPGASVKVSCKASGYTFTSYNMHWIRQAP |
| | | GQGLEWIGDIYPGSGDTSYNQKFKGRATLTADKSTSTVYMEL |
| | | SSLRSEDTAVYYCARVGGAFPMDYWGQGTLVTVSS |
| SEQ ID NO: 45 | DNA VH | CAGGTGCAGCTGGTGCAGTCAGGCGCCGAAGTGAAGAAA |
| | | CCCGGCGCAAGCGTTAAAGTCTCATGTAAAGCTAGTGGCT |
| | | ACACCTTCACTAGCTATAATATGCACTGGATTAGACAGGCC |
| | | CCAGGGCAAGGCCTGGAGTGGATCGGCGATATCTACCCC |
| | | GGTAGCGGCGACACTAGTTATAATCAGAAGTTTAAGGGTA |
| | | GAGCTACCCTGACCGCCGATAAGTCTACTAGCACCGTCTA |
| | | TATGGAACTGAGTTCCCTGAGGAGTGAAGACACCGCCGTC |
| | | TACTACTGCGCTAGAGTGGGCGGAGCCTTCCCTATGGACT |
| | | ACTGGGGTCAAGGCACCCTGGTCACCGTGTCAAGC |
| SEQ ID NO: 46 | Heavy | QVQLVQSGAEVKKPGASVKVSCKASGYTFTSYNMHWIRQAP |
| | Chain | GQGLEWIGDIYPGSGDTSYNQKFKGRATLTADKSTSTVYMEL |
| | | SSLRSEDTAVYYCARVGGAFPMDYWGQGTLVTVSSASTKGP |
| | | SVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQE |
| | | GNVFSCSVMHEALHNHYTQKSLSLSLG |
| SEQ ID NO: 47 | DNA | CAGGTGCAGCTGGTGCAGTCAGGCGCCGAAGTGAAGAAA |
| | Heavy | CCCGGCGCAAGCGTTAAAGTCTCATGTAAAGCTAGTGGCT |
| | Chain | ACACCTTCACTAGCTATAATATGCACTGGATTAGACAGGCC |
| | | CCAGGGCAAGGCCTGGAGTGGATCGGCGATATCTACCCC |
| | | GGTAGCGGCGACACTAGTTATAATCAGAAGTTTAAGGGTA |
| | | GAGCTACCCTGACCGCCGATAAGTCTACTAGCACCGTCTA |
| | | TATGGAACTGAGTTCCCTGAGGAGTGAAGACACCGCCGTC |
| | | TACTACTGCGCTAGAGTGGGCGGAGCCTTCCCTATGGACT |
| | | ACTGGGGTCAAGGCACCCTGGTCACCGTGTCAAGCGCTAG |
| | | CACTAAGGGCCCGTCCGTGTTCCCCCTGGCACCTTGTAGC |
| | | CGGAGCACTAGCGAATCCACCGCTGCCCTCGGCTGCCTG |
| | | GTCAAGGATTACTTCCCGGAGCCCGTGACCGTGTCCTGGA |
| | | ACAGCGGAGCCCTGACCTCCGGAGTGCACACCTTCCCCG |
| | | CTGTGCTGCAGAGCTCCGGGCTGTACTCGCTGTCGTCGGT |
| | | GGTCACGGTGCCTTCATCTAGCCTGGGTACCAAGACCTAC |
| | | ACTTGCAACGTGGACCACAAGCCTTCCAACACTAAGGTGG |
| | | ACAAGCGCGTCGAATCGAAGTACGGCCCACCGTGCCCGC |
| | | CTTGTCCCGCGCCGGAGTTCCTCGGCGGTCCCTCGGTCTT |
| | | TCTGTTCCCACCGAAGCCCAAGGACACTTTGATGATTTCCC |
| | | GCACCCCTGAAGTGACATGCGTGGTCGTGGACGTGTCACA |
| | | GGAAGATCCGGAGGTGCAGTTCAATTGGTACGTGGATGGC |
| | | GTCGAGGTGCACAACGCCAAAACCAAGCCGAGGGAGGAG |
| | | CAGTTCAACTCCACTTACCGCGTCGTGTCCGTGCTGACGG |
| | | TGCTGCATCAGGACTGGCTGAACGGGAAGGAGTACAAGTG |
| | | CAAAGTGTCCAACAAGGGACTTCCTAGCTCAATCGAAAAGA |
| | | CCATCTCGAAAGCCAAGGGACAGCCCCGGGAACCCCAAGT |
| | | GTATACCCTGCCACCGAGCCAGGAAGAAATGACTAAGAAC |
| | | CAAGTCTCATTGACTTGCCTTGTGAAGGGCTTCTACCCATC |
| | | GGATATCGCCGTGGAATGGGAGTCCAACGGCCAGCCGGA |
| | | AAACAACTACAAGACCACCCCTCCGGTGCTGGACTCAGAC |
| | | GGATCCTTCTTCCTCTACTCGCGGCTGACCGTGGATAAGA |
| | | GCAGATGGCAGGAGGGAAATGTGTTCAGCTGTTCTGTGAT |
| | | GCATGAAGCCCTGCACAACCACTACACTCAGAAGTCCCTG |
| | | TCCCTCTCCCTGGGA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 40 | VL | DIVLTQSPDSLAVSLGERATINCRASESVEYYGTSLMQWYQQ |
| | | KPGQPPKLLIYAASNVESGVPDRFSGSGSGTDFTLTISSLQAE |
| | | DVAVYFCQQSRKDPSTFGGGTKVEIK |
| SEQ ID NO: 41 | DNA VL | GATATCGTCCTGACTCAGTCACCCGATAGCCTGGCCGTCA |
| | | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTTCTGTCAGC |
| | | AGTCTAGGAAGGACCCTAGCACCTTCGGCGGAGGCACTAA |
| | | GGTCGAGATTAAG |
| SEQ ID NO: 42 | Light | DIVLTQSPDSLAVSLGERATINCRASESVEYYGTSLMQWYQQ |
| | Chain | KPGQPPKLLIYAASNVESGVPDRFSGSGSGTDFTLTISSLQAE |
| | | DVAVYFCQQSRKDPSTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 43 | DNA Light | GATATCGTCCTGACTCAGTCACCCGATAGCCTGGCCGTCA |
| | Chain | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTTCTGTCAGC |
| | | AGTCTAGGAAGGACCCTAGCACCTTCGGCGGAGGCACTAA |
| | | GGTCGAGATTAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum06 | | |
| SEQ ID NO: 3 | HCDR1 | SYNMH |
| (Kabat) | | |
| SEQ ID NO: 30 | HCDR2 | DIYPGQGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 31 | HCDR2 | YPGQGD |
| (Chothia) | | |
| SEQ ID NO: 5 | HCDR3 | VGGAFPMDY |
| (Chothia) | | |
| SEQ ID NO: 48 | VH | QVQLVQSGAEVKKPGASVKVSCKASGYTFTSYNMHWIRQAP |
| | | GQGLEWIGDIYPGQGDTSYNQKFKGRATLTADKSTSTVYMEL |
| | | SSLRSEDTAVYYCARVGGAFPMDYWGQGTLVTVSS |
| SEQ ID NO: 49 | DNA VH | CAGGTGCAGCTGGTGCAGTCAGGCGCCGAAGTGAAGAAA |
| | | CCCGGCGCTAGTGTGAAAGTCTCTTGTAAAGCTAGTGGCT |
| | | ACACCTTCACTAGCTATAATATGCACTGGATTAGACAGGCC |
| | | CCAGGTCAAGGCCTCGAGTGGATCGGCGATATCTACCCCG |
| | | GTCAAGGCGACACTAGTTATAATCAGAAGTTTAAGGGTAGA |
| | | GCTACCCTGACCGCCGATAAGTCTACTAGCACCGTCTATAT |
| | | GGAACTGAGTTCCCTGAGGTCTGAGGACACCGCCGTCTAC |
| | | TACTGCGCTAGAGTGGGCGGAGCCTTCCCTATGGACTACT |
| | | GGGGTCAAGGCACCCTGGTCACCGTGTCTAGC |
| SEQ ID NO: 50 | Heavy | QVQLVQSGAEVKKPGASVKVSCKASGYTFTSYNMHWIRQAP |
| | Chain | GQGLEWIGDIYPGQGDTSYNQKFKGRATLTADKSTSTVYMEL |
| | | SSLRSEDTAVYYCARVGGAFPMDYWGQGTLVTVSSASTKGP |
| | | SVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQE |
| | | GNVFSCSVMHEALHNHYTQKSLSLSLG |
| SEQ ID NO: 51 | DNA | CAGGTGCAGCTGGTGCAGTCAGGCGCCGAAGTGAAGAAA |
| | Heavy | CCCGGCGCTAGTGTGAAAGTCTCTTGTAAAGCTAGTGGCT |
| | Chain | ACACCTTCACTAGCTATAATATGCACTGGATTAGACAGGCC |
| | | CCAGGTCAAGGCCTCGAGTGGATCGGCGATATCTACCCCG |
| | | GTCAAGGCGACACTAGTTATAATCAGAAGTTTAAGGGTAGA |
| | | GCTACCCTGACCGCCGATAAGTCTACTAGCACCGTCTATAT |
| | | GGAACTGAGTTCCCTGAGGTCTGAGGACACCGCCGTCTAC |
| | | TACTGCGCTAGAGTGGGCGGAGCCTTCCCTATGGACTACT |
| | | GGGGTCAAGGCACCCTGGTCACCGTGTCTAGCGCTAGCAC |
| | | TAAGGGCCCGTCCGTGTTCCCCCTGGCACCTTGTAGCCGG |
| | | AGCACTAGCGAATCCACCGCTGCCCTCGGCTGCCTGGTCA |
| | | AGGATTACTTCCCGGAGCCCGTGACCGTGTCCTGGAACAG |
| | | CGGAGCCCTGACCTCCGGAGTGCACACCTTCCCCGCTGTG |
| | | CTGCAGAGCTCCGGGCTGTACTCGCTGTCGTCGGTGGTCA |
| | | CGGTGCCTTCATCTAGCCTGGGTACCAAGACCTACACTTG |
| | | CAACGTGGACCACAAGCCTTCCAACACTAAGGTGGACAAG |
| | | CGCGTCGAATCGAAGTACGGCCCACCGTGCCCGCCTTGTC |
| | | CCGCGCCGGAGTTCCTCGGCGGTCCCTCGGTCTTTCTGTT |
| | | CCCACCGAAGCCCAAGGACACTTTGATGATTTCCCGCACC |
| | | CCTGAAGTGACATGCGTGGTCGTGGACGTGTCACAGGAAG |
| | | ATCCGGAGGTGCAGTTCAATTGGTACGTGGATGGCGTCGA |
| | | GGTGCACAACGCCAAAACCAAGCCGAGGGAGGAGCAGTT |
| | | CAACTCCACTTACCGCGTCGTGTCCGTGCTGACGGTGCTG |
| | | CATCAGGACTGGCTGAACGGGAAGGAGTACAAGTGCAAAG |
| | | TGTCCAACAAGGGACTTCCTAGCTCAATCGAAAAGACCATC |
| | | TCGAAAGCCAAGGGACAGCCCCGGGAACCCCAAGTGTATA |
| | | CCCTGCCACCGAGCCAGGAAGAAATGACTAAGAACCAAGT |
| | | CTCATTGACTTGCCTTGTGAAGGGCTTCTACCCATCGGATA |
| | | TCGCCGTGGAATGGGAGTCCAACGGCCAGCCGGAAAACA |
| | | ACTACAAGACCACCCCTCCGGTGCTGGACTCAGACGGATC |
| | | CTTCTTCCTCTACTCGCGGCTGACCGTGGATAAGAGCAGA |
| | | TGGCAGGAGGGAAATGTGTTCAGCTGTTCTGTGATGCATG |
| | | AAGCCCTGCACAACCACTACACTCAGAAGTCCCTGTCCCT |
| | | CTCCCTGGGA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 40 | VL | DIVLTQSPDSLAVSLGERATINCRASESVEYYGTSLMQWYQQ |
| | | KPGQPPKLLIYAASNVESGVPDRFSGSGSGTDFTLTISSLQAE |
| | | DVAVYFCQQSRKDPSTFGGGTKVEIK |
| SEQ ID NO: 41 | DNA VL | GATATCGTCCTGACTCAGTCACCCGATAGCCTGGCCGTCA |
| | | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTTCTGTCAGC |
| | | AGTCTAGGAAGGACCCTAGCACCTTCGGCGGAGGCACTAA |
| | | GGTCGAGATTAAG |
| SEQ ID NO: 42 | Light | DIVLTQSPDSLAVSLGERATINCRASESVEYYGTSLMQWYQQ |
| | Chain | KPGQPPKLLIYAASNVESGVPDRFSGSGSGTDFTLTISSLQAE |
| | | DVAVYFCQQSRKDPSTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 43 | DNA Light | GATATCGTCCTGACTCAGTCACCCGATAGCCTGGCCGTCA |
| | Chain | GCCTGGGCGAGCGGGCTACTATTAACTGTAGAGCTAGTGA |
| | | ATCAGTCGAGTACTACGGCACTAGCCTGATGCAGTGGTAT |
| | | CAGCAGAAGCCCGGTCAACCCCCTAAGCTGCTGATCTACG |
| | | CCGCCTCTAACGTGGAATCAGGCGTGCCCGATAGGTTTAG |
| | | CGGTAGCGGTAGTGGCACCGACTTCACCCTGACTATTAGT |
| | | AGCCTGCAGGCCGAGGACGTGGCCGTCTACTTCTGTCAGC |
| | | AGTCTAGGAAGGACCCTAGCACCTTCGGCGGAGGCACTAA |
| | | GGTCGAGATTAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum07 | | |
| SEQ ID NO: 3 | HCDR1 | SYNMH |
| (Kabat) | | |
| SEQ ID NO: 4 | HCDR2 | DIYPGNGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 10 | HCDR2 | YPGNGD |
| (Chothia) | | |
| SEQ ID NO: 5 | HCDR3 | VGGAFPMDY |
| (Chothia) | | |
| SEQ ID NO: 36 | VH | QVQLVQSGAEVKKPGASVKVSCKASGYTFTSYNMHWIRQAP |
| | | GQGLEWIGDIYPGNGDTSYNQKFKGRATLTADKSTSTVYMEL |
| | | SSLRSEDTAVYYCARVGGAFPMDYWGQGTLVTVSS |
| SEQ ID NO: | DNA VH | CAGGTCCAGCTGGTCCAGAGCGGAGCAGAGGTCAAAAAG |
| 115 | | CCCGGAGCAAGCGTGAAGGTCTCATGCAAAGCAAGCGGAT |
| | | ACACATTTACATCATACAACATGCACTGGATCAGGCAGGCT |
| | | CCAGGACAGGGACTGGAGTGGATCGGGGACATCTACCCT |
| | | GGAAACGGCGATACTAGCTATAATCAGAAGTTCAAAGGCC |
| | | GGGCCACCCTGACAGCTGACAAGTCTACTAGTACCGTGTA |
| | | TATGGAGCTGAGCTCCCTGCGGTCTGAAGATACCGCAGTG |
| | | TACTATTGCGCCAGAGTCGGGGGGGCATTTCCTATGGATT |
| | | ATTGGGGGCAGGGGACTCTGGTCACTGTCTCCTCC |
| SEQ ID NO: | Heavy | QVQLVQSGAEVKKPGASVKVSCKASGYTFTSYNMHWIRQAP |
| 116 | Chain | GQGLEWIGDIYPGNGDTSYNQKFKGRATLTADKSTSTVYMEL |
| | | SSLRSEDTAVYYCARVGGAFPMDYWGQGTLVTVSSASTKGP |
| | | SVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQE |
| | | GNVFSCSVMHEALHNHYTQKSLSLSLGK |
| SEQ ID NO: | DNA | CAGGTCCAGCTGGTCCAGAGCGGAGCAGAGGTCAAAAAG |
| 117 | Heavy | CCCGGAGCAAGCGTGAAGGTCTCATGCAAAGCAAGCGGAT |
| | Chain | ACACATTTACATCATACAACATGCACTGGATCAGGCAGGCT |
| | | CCAGGACAGGGACTGGAGTGGATCGGGGACATCTACCCT |
| | | GGAAACGGCGATACTAGCTATAATCAGAAGTTCAAAGGCC |
| | | GGGCCACCCTGACAGCTGACAAGTCTACTAGTACCGTGTA |
| | | TATGGAGCTGAGCTCCCTGCGGTCTGAAGATACCGCAGTG |
| | | TACTATTGCGCCAGAGTCGGGGGGGCATTTCCTATGGATT |
| | | ATTGGGGGCAGGGGACTCTGGTCACTGTCTCCTCCGCTAG |
| | | CACCAAGGGCCCATCCGTCTTCCCCCTGGCGCCCTGCTCC |
| | | AGGAGCACCTCCGAGAGCACAGCCGCCCTGGGCTGCCTG |
| | | GTCAAGGACTACTTCCCCGAACCGGTGACGGTGTCGTGGA |
| | | ACTCAGGCGCCCTGACCAGCGGCGTGCACACCTTCCCGG |
| | | CTGTCCTACAGTCCTCAGGACTCTACTCCCTCAGCAGCGT |
| | | GGTGACCGTGCCCTCCAGCAGCTTGGGCACGAAGACCTAC |
| | | ACCTGCAACGTAGATCACAAGCCCAGCAACACCAAGGTGG |
| | | ACAAGAGAGTTGAGTCCAAATATGGTCCCCCATGCCCACC |
| | | ATGCCCAGCACCTGAGTTCCTGGGGGGACCATCAGTCTTC |
| | | CTGTTCCCCCCAAAACCCAAGGACACTCTCATGATCTCCCG |
| | | GACCCCTGAGGTCACGTGCGTGGTGGTGGACGTGAGCCA |
| | | GGAAGACCCCGAGGTCCAGTTCAACTGGTACGTGGATGGC |
| | | GTGGAGGTGCATAATGCCAAGACAAAGCCGCGGGAGGAG |
| | | CAGTTCAACAGCACGTACCGTGTGGTCAGCGTCCTCACCG |
| | | TCCTGCACCAGGACTGGCTGAACGGCAAGGAGTACAAGTG |
| | | CAAGGTCTCCAACAAAGGCCTCCCGTCCTCCATCGAGAAA |
| | | ACCATCTCCAAAGCCAAAGGGCAGCCCCGAGAGCCACAG |
| | | GTGTACACCCTGCCCCCATCCCAGGAGGAGATGACCAAGA |
| | | ACCAGGTCAGCCTGACCTGCCTGGTCAAAGGCTTCTACCC |
| | | CAGCGACATCGCCGTGGAGTGGGAGAGCAATGGGCAGCC |
| | | GGAGAACAACTACAAGACCACGCCTCCCGTGCTGGACTCC |
| | | GACGGCTCCTTCTTCCTCTACAGCAGGCTAACCGTGGACA |
| | | AGAGCAGGTGGCAGGAGGGGAATGTCTTCTCATGCTCCGT |
| | | GATGCATGAGGCTCTGCACAACCACTACACACAGAAGAGC |
| | | CTCTCCCTGTCTCTGGGTAAA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 20 | VL | DIVLTQSPDSLAVSLGERATINCRASESVEYYGTSLMQWYQQ |
| | | KPGQPPKLLIYAASNVESGVPDRFSGSGSGTDFTLTISSLQAE |
| | | DVAVYYCQQSRKDPSTFGGGTKVEIK |
| SEQ ID NO: | DNA VL | GACATCGTCCTGACACAGTCTCCTGACAGCCTGGCAGTGA |
| 118 | | GCCTGGGCGAAAGGGCAACCATTAATTGTAGAGCTTCCGA |
| | | GTCCGTCGAGTACTATGGCACTAGTCTGATGCAGTGGTAC |
| | | CAGCAGAAGCCAGGGCAGCCCCCTAAACTGCTGATCTATG |
| | | CAGCTAGCAACGTGGAGTCCGGAGTCCCAGACCGGTTCTC |
| | | TGGAAGTGGGTCAGGAACCGATTTTACCCTGACAATTAGCT |
| | | CCCTGCAGGCAGAAGACGTGGCCGTCTACTATTGTCAGCA |
| | | GAGCCGCAAGGACCCAAGCACATTCGGAGGGGGGACCAA |
| | | AGTGGAAATCAAG |
| SEQ ID NO: 22 | Light | DIVLTQSPDSLAVSLGERATINCRASESVEYYGTSLMQWYQQ |
| | Chain | KPGQPPKLLIYAASNVESGVPDRFSGSGSGTDFTLTISSLQAE |
| | | DVAVYYCQQSRKDPSTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: | DNA Light | GACATCGTCCTGACACAGTCTCCTGACAGCCTGGCAGTGA |
| 119 | Chain | GCCTGGGCGAAAGGGCAACCATTAATTGTAGAGCTTCCGA |
| | | GTCCGTCGAGTACTATGGCACTAGTCTGATGCAGTGGTAC |
| | | CAGCAGAAGCCAGGGCAGCCCCCTAAACTGCTGATCTATG |
| | | CAGCTAGCAACGTGGAGTCCGGAGTCCCAGACCGGTTCTC |
| | | TGGAAGTGGGTCAGGAACCGATTTTACCCTGACAATTAGCT |
| | | CCCTGCAGGCAGAAGACGTGGCCGTCTACTATTGTCAGCA |
| | | GAGCCGCAAGGACCCAAGCACATTCGGAGGGGGGACCAA |
| | | AGTGGAAATCAAGCGGACTGTTGCTGCACCATCTGTCTTCA |
| | | TCTTCCCGCCATCTGATGAGCAGTTGAAATCTGGAACTGCC |
| | | TCTGTTGTGTGCCTGCTGAATAACTTCTATCCCAGAGAGGC |
| | | CAAAGTACAGTGGAAGGTGGATAACGCCCTCCAATCGGGT |
| | | AACTCCCAGGAGAGTGTCACAGAGCAGGACAGCAAGGACA |
| | | GCACCTACAGCCTCAGCAGCACCCTGACGCTGAGCAAAGC |
| | | AGACTACGAGAAACACAAAGTCTACGCCTGCGAAGTCACC |
| | | CATCAGGGCCTGAGTTCACCGGTGACAAAGAGCTTCAACA |
| | | GGGGAGAGTGT |
| ABTIM3- | | |
| hum08 | | |
| SEQ ID NO: 3 | HCDR1 | SYNMH |
| (Kabat) | | |
| SEQ ID NO: 4 | HCDR2 | DIYPGNGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 10 | HCDR2 | YPGNGD |
| (Chothia) | | |
| SEQ ID NO: 5 | HCDR3 | VGGAFPMDY |
| (Chothia) | | |
| SEQ ID NO: 16 | VH | QVQLVQSGAEVKKPGASVKVSCKASGYTFTSYNMHWVRQA |
| | | PGQGLEWIGDIYPGNGDTSYNQKFKGRATMTADKSTSTVYM |
| | | ELSSLRSEDTAVYYCARVGGAFPMDYWGQGTLVTVSS |
| SEQ ID NO: | DNA VH | CAGGTCCAGCTGGTCCAGAGCGGAGCAGAGGTCAAAAAG |
| 120 | | CCCGGAGCAAGCGTGAAGGTCTCATGCAAAGCAAGCGGAT |
| | | ACACATTTACATCATACAACATGCACTGGGTCAGGCAGGCT |
| | | CCAGGACAGGGACTGGAGTGGATCGGGGACATCTACCCT |
| | | GGAAACGGCGATACTAGCTATAATCAGAAGTTCAAAGGCC |
| | | GGGCCACCATGACAGCTGACAAGTCTACTAGTACCGTGTA |
| | | TATGGAGCTGAGCTCCCTGCGGTCTGAAGATACCGCAGTG |
| | | TACTATTGCGCCAGAGTCGGGGGGGCATTTCCTATGGATT |
| | | ATTGGGGGCAGGGGACTCTGGTCACTGTCTCCTCC |
| SEQ ID NO: | Heavy | QVQLVQSGAEVKKPGASVKVSCKASGYTFTSYNMHWVRQA |
| 121 | Chain | PGQGLEWIGDIYPGNGDTSYNQKFKGRATMTADKSTSTVYM |
| | | ELSSLRSEDTAVYYCARVGGAFPMDYWGQGTLVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPS |
| | | DIAVEWESNGQPENNYKTTPPVLDSDGSFFLYSRLTVDKSRW |
| | | QEGNVFSCSVMHEALHNHYTQKSLSLSLGK |
| SEQ ID NO: | DNA | CAGGTCCAGCTGGTCCAGAGCGGAGCAGAGGTCAAAAAG |
| 122 | Heavy | CCCGGAGCAAGCGTGAAGGTCTCATGCAAAGCAAGCGGAT |
| | Chain | ACACATTTACATCATACAACATGCACTGGGTCAGGCAGGCT |
| | | CCAGGACAGGGACTGGAGTGGATCGGGGACATCTACCCT |
| | | GGAAACGGCGATACTAGCTATAATCAGAAGTTCAAAGGCC |
| | | GGGCCACCATGACAGCTGACAAGTCTACTAGTACCGTGTA |
| | | TATGGAGCTGAGCTCCCTGCGGTCTGAAGATACCGCAGTG |
| | | TACTATTGCGCCAGAGTCGGGGGGGCATTTCCTATGGATT |
| | | ATTGGGGGCAGGGGACTCTGGTCACTGTCTCCTCCGCTAG |
| | | CACCAAGGGCCCATCCGTCTTCCCCCTGGCGCCCTGCTCC |
| | | AGGAGCACCTCCGAGAGCACAGCCGCCCTGGGCTGCCTG |
| | | GTCAAGGACTACTTCCCCGAACCGGTGACGGTGTCGTGGA |
| | | ACTCAGGCGCCCTGACCAGCGGCGTGCACACCTTCCCGG |
| | | CTGTCCTACAGTCCTCAGGACTCTACTCCCTCAGCAGCGT |
| | | GGTGACCGTGCCCTCCAGCAGCTTGGGCACGAAGACCTAC |
| | | ACCTGCAACGTAGATCACAAGCCCAGCAACACCAAGGTGG |
| | | ACAAGAGAGTTGAGTCCAAATATGGTCCCCCATGCCCACC |
| | | ATGCCCAGCACCTGAGTTCCTGGGGGGACCATCAGTCTTC |
| | | CTGTTCCCCCCAAAACCCAAGGACACTCTCATGATCTCCCG |
| | | GACCCCTGAGGTCACGTGCGTGGTGGTGGACGTGAGCCA |
| | | GGAAGACCCCGAGGTCCAGTTCAACTGGTACGTGGATGGC |
| | | GTGGAGGTGCATAATGCCAAGACAAAGCCGCGGGAGGAG |
| | | CAGTTCAACAGCACGTACCGTGTGGTCAGCGTCCTCACCG |
| | | TCCTGCACCAGGACTGGCTGAACGGCAAGGAGTACAAGTG |
| | | CAAGGTCTCCAACAAAGGCCTCCCGTCCTCCATCGAGAAA |
| | | ACCATCTCCAAAGCCAAAGGGCAGCCCCGAGAGCCACAG |
| | | GTGTACACCCTGCCCCCATCCCAGGAGGAGATGACCAAGA |
| | | ACCAGGTCAGCCTGACCTGCCTGGTCAAAGGCTTCTACCC |
| | | CAGCGACATCGCCGTGGAGTGGGAGAGCAATGGGCAGCC |
| | | GGAGAACAACTACAAGACCACGCCTCCCGTGCTGGACTCC |
| | | GACGGCTCCTTCTTCCTCTACAGCAGGCTAACCGTGGACA |
| | | AGAGCAGGTGGCAGGAGGGGAATGTCTTCTCATGCTCCGT |
| | | GATGCATGAGGCTCTGCACAACCACTACACACAGAAGAGC |
| | | CTCTCCCTGTCTCTGGGTAAA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 40 | VL | DIVLTQSPDSLAVSLGERATINCRASESVEYYGTSLMQWYQQ |
| | | KPGQPPKLLIYAASNVESGVPDRFSGSGSGTDFTLTISSLQAE |
| | | DVAVYFCQQSRKDPSTFGGGTKVEIK |
| SEQ ID NO: | DNA VL | GACATCGTCCTGACACAGTCTCCTGACAGCCTGGCAGTGA |
| 123 | | GCCTGGGCGAAAGGGCAACCATTAATTGTAGAGCTTCCGA |
| | | GTCCGTCGAGTACTATGGCACTAGTCTGATGCAGTGGTAC |
| | | CAGCAGAAGCCAGGGCAGCCCCCTAAACTGCTGATCTATG |
| | | CAGCTAGCAACGTGGAGTCCGGAGTCCCAGACCGGTTCTC |
| | | TGGAAGTGGGTCAGGAACCGATTTTACCCTGACAATTAGCT |
| | | CCCTGCAGGCAGAAGACGTGGCCGTCTACTTTTGTCAGCA |
| | | GAGCCGCAAGGACCCAAGCACATTCGGAGGGGGGACCAA |
| | | AGTGGAAATCAAG |
| SEQ ID NO: 42 | Light | DIVLTQSPDSLAVSLGERATINCRASESVEYYGTSLMQWYQQ |
| | Chain | KPGQPPKLLIYAASNVESGVPDRFSGSGSGTDFTLTISSLQAE |
| | | DVAVYFCQQRKDPSTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: | DNA Light | GACATCGTCCTGACACAGTCTCCTGACAGCCTGGCAGTGA |
| 124 | Chain | GCCTGGGCGAAAGGGCAACCATTAATTGTAGAGCTTCCGA |
| | | GTCCGTCGAGTACTATGGCACTAGTCTGATGCAGTGGTAC |
| | | CAGCAGAAGCCAGGGCAGCCCCCTAAACTGCTGATCTATG |
| | | CAGCTAGCAACGTGGAGTCCGGAGTCCCAGACCGGTTCTC |
| | | TGGAAGTGGGTCAGGAACCGATTTTACCCTGACAATTAGCT |
| | | CCCTGCAGGCAGAAGACGTGGCCGTCTACTTTTGTCAGCA |
| | | GAGCCGCAAGGACCCAAGCACATTCGGAGGGGGGACCAA |
| | | AGTGGAAATCAAGCGGACTGTTGCTGCACCATCTGTCTTCA |
| | | TCTTCCCGCCATCTGATGAGCAGTTGAAATCTGGAACTGCC |
| | | TCTGTTGTGTGCCTGCTGAATAACTTCTATCCCAGAGAGGC |
| | | CAAAGTACAGTGGAAGGTGGATAACGCCCTCCAATCGGGT |
| | | AACTCCCAGGAGAGTGTCACAGAGCAGGACAGCAAGGACA |
| | | GCACCTACAGCCTCAGCAGCACCCTGACGCTGAGCAAAGC |
| | | AGACTACGAGAAACACAAAGTCTACGCCTGCGAAGTCACC |
| | | CATCAGGGCCTGAGTTCACCGGTGACAAAGAGCTTCAACA |
| | | GGGGAGAGTGT |
| ABTIM3- | | |
| hum09 | | |
| SEQ ID NO: 3 | HCDR1 | SYNMH |
| (Kabat) | | |
| SEQ ID NO: 4 | HCDR2 | DIYPGNGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 10 | HCDR2 | YPGNGD |
| (Chothia) | | |
| SEQ ID NO: 5 | HCDR3 | VGGAFPMDY |
| (Chothia) | | |
| SEQ ID NO: 52 | VH | QVQLVQSGAEVKKPGSSVKVSCKASGYTFTSYNMHWVRQA |
| | | PGQGLEWMGDIYPGNGDTSYNQKFKGRVTITADKSTSTVYM |
| | | ELSSLRSEDTAVYYCARVGGAFPMDYWGQGTTVTVSS |
| SEQ ID NO: 53 | DNA VH | CAGGTGCAGCTGGTGCAGTCAGGCGCCGAAGTGAAGAAA |
| | | CCCGGCTCTAGCGTGAAAGTTTCTTGTAAAGCTAGTGGCTA |
| | | CACCTTCACTAGCTATAATATGCACTGGGTTCGCCAGGCCC |
| | | CAGGGCAAGGCCTCGAGTGGATGGGCGATATCTACCCCG |
| | | GGAACGGCGACACTAGTTATAATCAGAAGTTTAAGGGTAGA |
| | | GTCACTATCACCGCCGATAAGTCTACTAGCACCGTCTATAT |
| | | GGAACTGAGTTCCCTGAGGTCTGAGGACACCGCCGTCTAC |
| | | TACTGCGCTAGAGTGGGCGGAGCCTTCCCTATGGACTACT |
| | | GGGGTCAAGGCACTACCGTGACCGTGTCTAGC |
| SEQ ID NO: 54 | Heavy | QVQLVQSGAEVKKPGSSVKVSCKASGYTFTSYNMHWVRQA |
| | Chain | PGQGLEWMGDIYPGNGDTSYNQKFKGRVTITADKSTSTVYM |
| | | ELSSLRSEDTAVYYCARVGGAFPMDYWGQGTTVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPS |
| | | DIAVEWESNGQPENNYKTTPPVLDSDGSFFLYSRLTVDKSRW |
| | | QEGNVFSCSVMHEALHNHYTQKSLSLSLG |
| SEQ ID NO: 55 | DNA | CAGGTGCAGCTGGTGCAGTCAGGCGCCGAAGTGAAGAAA |
| | Heavy | CCCGGCTCTAGCGTGAAAGTTTCTTGTAAAGCTAGTGGCTA |
| | Chain | CACCTTCACTAGCTATAATATGCACTGGGTTCGCCAGGCCC |
| | | CAGGGCAAGGCCTCGAGTGGATGGGCGATATCTACCCCG |
| | | GGAACGGCGACACTAGTTATAATCAGAAGTTTAAGGGTAGA |
| | | GTCACTATCACCGCCGATAAGTCTACTAGCACCGTCTATAT |
| | | GGAACTGAGTTCCCTGAGGTCTGAGGACACCGCCGTCTAC |
| | | TACTGCGCTAGAGTGGGCGGAGCCTTCCCTATGGACTACT |
| | | GGGGTCAAGGCACTACCGTGACCGTGTCTAGCGCTAGCAC |
| | | TAAGGGCCCGTCCGTGTTCCCCCTGGCACCTTGTAGCCGG |
| | | AGCACTAGCGAATCCACCGCTGCCCTCGGCTGCCTGGTCA |
| | | AGGATTACTTCCCGGAGCCCGTGACCGTGTCCTGGAACAG |
| | | CGGAGCCCTGACCTCCGGAGTGCACACCTTCCCCGCTGTG |
| | | CTGCAGAGCTCCGGGCTGTACTCGCTGTCGTCGGTGGTCA |
| | | CGGTGCCTTCATCTAGCCTGGGTACCAAGACCTACACTTG |
| | | CAACGTGGACCACAAGCCTTCCAACACTAAGGTGGACAAG |
| | | CGCGTCGAATCGAAGTACGGCCCACCGTGCCCGCCTTGTC |
| | | CCGCGCCGGAGTTCCTCGGCGGTCCCTCGGTCTTTCTGTT |
| | | CCCACCGAAGCCCAAGGACACTTTGATGATTTCCCGCACC |
| | | CCTGAAGTGACATGCGTGGTCGTGGACGTGTCACAGGAAG |
| | | ATCCGGAGGTGCAGTTCAATTGGTACGTGGATGGCGTCGA |
| | | GGTGCACAACGCCAAAACCAAGCCGAGGGAGGAGCAGTT |
| | | CAACTCCACTTACCGCGTCGTGTCCGTGCTGACGGTGCTG |
| | | CATCAGGACTGGCTGAACGGGAAGGAGTACAAGTGCAAAG |
| | | TGTCCAACAAGGGACTTCCTAGCTCAATCGAAAAGACCATC |
| | | TCGAAAGCCAAGGGACAGCCCCGGGAACCCCAAGTGTATA |
| | | CCCTGCCACCGAGCCAGGAAGAAATGACTAAGAACCAAGT |
| | | CTCATTGACTTGCCTTGTGAAGGGCTTCTACCCATCGGATA |
| | | TCGCCGTGGAATGGGAGTCCAACGGCCAGCCGGAAAACA |
| | | ACTACAAGACCACCCCTCCGGTGCTGGACTCAGACGGATC |
| | | CTTCTTCCTCTACTCGCGGCTGACCGTGGATAAGAGCAGA |
| | | TGGCAGGAGGGAAATGTGTTCAGCTGTTCTGTGATGCATG |
| | | AAGCCCTGCACAACCACTACACTCAGAAGTCCCTGTCCCT |
| | | CTCCCTGGGA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 56 | VL | EIVLTQSPATLSLSPGERATLSCRASESVEYYGTSLMQWYQQ |
| | | KPGQAPRLLIYAASNVESGIPARFSGSGSGTDFTLTISSLEPED |
| | | IAVYFCQQSRKDPSTFGGGTKVEIK |
| SEQ ID NO: 5 | DNA VL | GAGATCGTCCTGACTCAGTCACCCGCTACCCTGAGCCTGA |
| | | GCCCTGGCGAGAGAGCTACACTGAGCTGTAGAGCTAGTGA |
| | | ATCAGTCGAGTACTACGGCACTAGCCTGATGCAGTGGTAT |
| | | CAGCAGAAGCCCGGTCAAGCCCCTAGACTGCTGATCTACG |
| | | CCGCCTCTAACGTGGAATCAGGGATCCCCGCTAGGTTTAG |
| | | CGGTAGCGGTAGTGGCACCGACTTCACCCTGACTATCTCT |
| | | AGCCTGGAACCCGAGGATATCGCCGTCTACTTCTGTCAGC |
| | | AGTCTAGGAAGGACCCTAGCACCTTCGGCGGAGGCACTAA |
| | | GGTCGAGATTAAG |
| SEQ ID NO: 58 | Light | EIVLTQSPATLSLSPGERATLSCRASESVEYYGTSLMQWYQQ |
| | Chain | KPGQAPRLLIYAASNVESGIPARFSGSGSGTDFTLTISSLEPED |
| | | IAVYFCQQSRKDPSTFGGGTKVEIKRTVAAPSVFIFPPSDEQL |
| | | KSGTASVVCLLNNFYPREAKVQWKVDNALQSGNSQESVTEQ |
| | | DSKDSTYSLSSTLTLSKADYEKHKVYACEVTHQGLSSPVTKSF |
| | | NRGEC |
| SEQ ID NO: 59 | DNA Light | GAGATCGTCCTGACTCAGTCACCCGCTACCCTGAGCCTGA |
| | Chain | GCCCTGGCGAGAGAGCTACACTGAGCTGTAGAGCTAGTGA |
| | | ATCAGTCGAGTACTACGGCACTAGCCTGATGCAGTGGTAT |
| | | CAGCAGAAGCCCGGTCAAGCCCCTAGACTGCTGATCTACG |
| | | CCGCCTCTAACGTGGAATCAGGGATCCCCGCTAGGTTTAG |
| | | CGGTAGCGGTAGTGGCACCGACTTCACCCTGACTATCTCT |
| | | AGCCTGGAACCCGAGGATATCGCCGTCTACTTCTGTCAGC |
| | | AGTCTAGGAAGGACCCTAGCACCTTCGGCGGAGGCACTAA |
| | | GGTCGAGATTAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum10 | | |
| SEQ ID NO: 3 | HCDR1 | SYNMH |
| (Kabat) | | |
| SEQ ID NO: 4 | HCDR2 | DIYPGNGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 10 | HCDR2 | YPGNGD |
| (Chothia) | | |
| SEQ ID NO: 5 | HCDR3 | VGGAFPMDY |
| (Chothia) | | |
| SEQ ID NO: 60 | VH | EVQLVQSGAEVKKPGESLKISCKGSGYTFTSYNMHWVRQMP |
| | | GKGLEWMGDIYPGNGDTSYNQKFKGQVTISADKSISTVYLQW |
| | | SSLKASDTAMYYCARVGGAFPMDYWGQGTTVTVSS |
| SEQ ID NO: 61 | DNA VH | GAAGTGCAGCTGGTGCAGTCAGGCGCCGAAGTGAAGAAG |
| | | CCCGGCGAGTCACTGAAGATTAGCTGTAAAGGTTCAGGCT |
| | | ACACCTTCACTAGCTATAATATGCACTGGGTCCGCCAGATG |
| | | CCCGGGAAAGGCCTCGAGTGGATGGGCGATATCTACCCC |
| | | GGGAACGGCGACACTAGTTATAATCAGAAGTTTAAGGGGC |
| | | AAGTCACAATTAGCGCCGATAAGTCTATTAGCACCGTCTAC |
| | | CTGCAGTGGTCTAGCCTGAAGGCTAGTGACACCGCTATGT |
| | | ACTACTGCGCTAGAGTGGGCGGAGCCTTCCCTATGGACTA |
| | | CTGGGGTCAAGGCACTACCGTGACCGTGTCTAGC |
| SEQ ID NO: 62 | Heavy | EVQLVQSGAEVKKPGESLKISCKGSGYTFTSYNMHWVRQMP |
| | Chain | GKGLEWMGDIYPGNGDTSYNQKFKGQVTISADKSISTVYLQW |
| | | SSLKASDTAMYYCARVGGAFPMDYWGQGTTVTVSSASTKGP |
| | | SVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQE |
| | | GNVFSCSVMHEALHNHYTQKSLSLSLG |
| SEQ ID NO: 63 | DNA | GAAGTGCAGCTGGTGCAGTCAGGCGCCGAAGTGAAGAAG |
| | Heavy | CCCGGCGAGTCACTGAAGATTAGCTGTAAAGGTTCAGGCT |
| | Chain | ACACCTTCACTAGCTATAATATGCACTGGGTCCGCCAGATG |
| | | CCCGGGAAAGGCCTCGAGTGGATGGGCGATATCTACCCC |
| | | GGGAACGGCGACACTAGTTATAATCAGAAGTTTAAGGGGC |
| | | AAGTCACAATTAGCGCCGATAAGTCTATTAGCACCGTCTAC |
| | | CTGCAGTGGTCTAGCCTGAAGGCTAGTGACACCGCTATGT |
| | | ACTACTGCGCTAGAGTGGGCGGAGCCTTCCCTATGGACTA |
| | | CTGGGGTCAAGGCACTACCGTGACCGTGTCTAGCGCTAGC |
| | | ACTAAGGGCCCGTCCGTGTTCCCCCTGGCACCTTGTAGCC |
| | | GGAGCACTAGCGAATCCACCGCTGCCCTCGGCTGCCTGGT |
| | | CAAGGATTACTTCCCGGAGCCCGTGACCGTGTCCTGGAAC |
| | | AGCGGAGCCCTGACCTCCGGAGTGCACACCTTCCCCGCT |
| | | GTGCTGCAGAGCTCCGGGCTGTACTCGCTGTCGTCGGTG |
| | | GTCACGGTGCCTTCATCTAGCCTGGGTACCAAGACCTACA |
| | | CTTGCAACGTGGACCACAAGCCTTCCAACACTAAGGTGGA |
| | | CAAGCGCGTCGAATCGAAGTACGGCCCACCGTGCCCGCC |
| | | TTGTCCCGCGCCGGAGTTCCTCGGCGGTCCCTCGGTCTTT |
| | | CTGTTCCCACCGAAGCCCAAGGACACTTTGATGATTTCCCG |
| | | CACCCCTGAAGTGACATGCGTGGTCGTGGACGTGTCACAG |
| | | GAAGATCCGGAGGTGCAGTTCAATTGGTACGTGGATGGCG |
| | | TCGAGGTGCACAACGCCAAAACCAAGCCGAGGGAGGAGC |
| | | AGTTCAACTCCACTTACCGCGTCGTGTCCGTGCTGACGGT |
| | | GCTGCATCAGGACTGGCTGAACGGGAAGGAGTACAAGTGC |
| | | AAAGTGTCCAACAAGGGACTTCCTAGCTCAATCGAAAAGAC |
| | | CATCTCGAAAGCCAAGGGACAGCCCCGGGAACCCCAAGT |
| | | GTATACCCTGCCACCGAGCCAGGAAGAAATGACTAAGAAC |
| | | CAAGTCTCATTGACTTGCCTTGTGAAGGGCTTCTACCCATC |
| | | GGATATCGCCGTGGAATGGGAGTCCAACGGCCAGCCGGA |
| | | AAACAACTACAAGACCACCCCTCCGGTGCTGGACTCAGAC |
| | | GGATCCTTCTTCCTCTACTCGCGGCTGACCGTGGATAAGA |
| | | GCAGATGGCAGGAGGGAAATGTGTTCAGCTGTTCTGTGAT |
| | | GCATGAAGCCCTGCACAACCACTACACTCAGAAGTCCCTG |
| | | TCCCTCTCCCTGGGA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 56 | VL | EIVLTQSPATLSLSPGERATLSCRASESVEYYGTSLMQWYQQ |
| | | KPGQAPRLLIYAASNVESGIPARFSGSGSGTDFTLTISSLEPED |
| | | IAVYFCQQSRKDPSTFGGGTKVEIK |
| SEQ ID NO: 57 | DNA VL | GAGATCGTCCTGACTCAGTCACCCGCTACCCTGAGCCTGA |
| | | GCCCTGGCGAGAGAGCTACACTGAGCTGTAGAGCTAGTGA |
| | | ATCAGTCGAGTACTACGGCACTAGCCTGATGCAGTGGTAT |
| | | CAGCAGAAGCCCGGTCAAGCCCCTAGACTGCTGATCTACG |
| | | CCGCCTCTAACGTGGAATCAGGGATCCCCGCTAGGTTTAG |
| | | CGGTAGCGGTAGTGGCACCGACTTCACCCTGACTATCTCT |
| | | AGCCTGGAACCCGAGGATATCGCCGTCTACTTCTGTCAGC |
| | | AGTCTAGGAAGGACCCTAGCACCTTCGGCGGAGGCACTAA |
| | | GGTCGAGATTAAG |
| SEQ ID NO: 58 | Light | EIVLTQSPATLSLSPGERATLSCRASESVEYYGTSLMQWYQQ |
| | Chain | KPGQAPRLLIYAASNVESGIPARFSGSGSGTDFTLTISSLEPED |
| | | IAVYFCQQSRKDPSTFGGGTKVEIKRTVAAPSVFIFPPSDEQL |
| | | KSGTASVVCLLNNFYPREAKVQWKVDNALQSGNSQESVTEQ |
| | | DSKDSTYSLSSTLTLSKADYEKHKVYACEVTHQGLSSPVTKSF |
| | | NRGEC |
| SEQ ID NO: 59 | DNA Light | GAGATCGTCCTGACTCAGTCACCCGCTACCCTGAGCCTGA |
| | Chain | GCCCTGGCGAGAGAGCTACACTGAGCTGTAGAGCTAGTGA |
| | | ATCAGTCGAGTACTACGGCACTAGCCTGATGCAGTGGTAT |
| | | CAGCAGAAGCCCGGTCAAGCCCCTAGACTGCTGATCTACG |
| | | CCGCCTCTAACGTGGAATCAGGGATCCCCGCTAGGTTTAG |
| | | CGGTAGCGGTAGTGGCACCGACTTCACCCTGACTATCTCT |
| | | AGCCTGGAACCCGAGGATATCGCCGTCTACTTCTGTCAGC |
| | | AGTCTAGGAAGGACCCTAGCACCTTCGGCGGAGGCACTAA |
| | | GGTCGAGATTAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum11 | | |
| SEQ ID NO: 3 | HCDR1 | SYNMH |
| (Kabat) | | |
| SEQ ID NO: 4 | HCDR2 | DIYPGNGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 10 | HCDR2 | YPGNGD |
| (Chothia) | | |
| SEQ ID NO: 5 | HCDR3 | VGGAFPMDY |
| (Chothia) | | |
| SEQ ID NO: 52 | VH | QVQLVQSGAEVKKPGSSVKVSCKASGYTFTSYNMHWVRQA |
| | | PGQGLEWMGDIYPGNGDTSYNQKFKGRVTITADKSTSTVYM |
| | | ELSSLRSEDTAVYYCARVGGAFPMDYWGQGTTVTVSS |
| SEQ ID NO: 53 | DNA VH | CAGGTGCAGCTGGTGCAGTCAGGCGCCGAAGTGAAGAAA |
| | | CCCGGCTCTAGCGTGAAAGTTTCTTGTAAAGCTAGTGGCTA |
| | | CACCTTCACTAGCTATAATATGCACTGGGTTCGCCAGGCCC |
| | | CAGGGCAAGGCCTCGAGTGGATGGGCGATATCTACCCCG |
| | | GGAACGGCGACACTAGTTATAATCAGAAGTTTAAGGGTAGA |
| | | GTCACTATCACCGCCGATAAGTCTACTAGCACCGTCTATAT |
| | | GGAACTGAGTTCCCTGAGGTCTGAGGACACCGCCGTCTAC |
| | | TACTGCGCTAGAGTGGGCGGAGCCTTCCCTATGGACTACT |
| | | GGGGTCAAGGCACTACCGTGACCGTGTCTAGC |
| SEQ ID NO: 54 | Heavy | QVQLVQSGAEVKKPGSSVKVSCKASGYTFTSYNMHWVRQA |
| | Chain | PGQGLEWMGDIYPGNGDTSYNQKFKGRVTITADKSTSTVYM |
| | | ELSSLRSEDTAVYYCARVGGAFPMDYWGQGTTVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPS |
| | | DIAVEWESNGQPENNYKTTPPVLDSDGSFFLYSRLTVDKSRW |
| | | QEGNVFSCSVMHEALHNHYTQKSLSLSLG |
| SEQ ID NO: 55 | DNA | CAGGTGCAGCTGGTGCAGTCAGGCGCCGAAGTGAAGAAA |
| | Heavy | CCCGGCTCTAGCGTGAAAGTTTCTTGTAAAGCTAGTGGCTA |
| | Chain | CACCTTCACTAGCTATAATATGCACTGGGTTCGCCAGGCCC |
| | | CAGGGCAAGGCCTCGAGTGGATGGGCGATATCTACCCCG |
| | | GGAACGGCGACACTAGTTATAATCAGAAGTTTAAGGGTAGA |
| | | GTCACTATCACCGCCGATAAGTCTACTAGCACCGTCTATAT |
| | | GGAACTGAGTTCCCTGAGGTCTGAGGACACCGCCGTCTAC |
| | | TACTGCGCTAGAGTGGGCGGAGCCTTCCCTATGGACTACT |
| | | GGGGTCAAGGCACTACCGTGACCGTGTCTAGCGCTAGCAC |
| | | TAAGGGCCCGTCCGTGTTCCCCCTGGCACCTTGTAGCCGG |
| | | AGCACTAGCGAATCCACCGCTGCCCTCGGCTGCCTGGTCA |
| | | AGGATTACTTCCCGGAGCCCGTGACCGTGTCCTGGAACAG |
| | | CGGAGCCCTGACCTCCGGAGTGCACACCTTCCCCGCTGTG |
| | | CTGCAGAGCTCCGGGCTGTACTCGCTGTCGTCGGTGGTCA |
| | | CGGTGCCTTCATCTAGCCTGGGTACCAAGACCTACACTTG |
| | | CAACGTGGACCACAAGCCTTCCAACACTAAGGTGGACAAG |
| | | CGCGTCGAATCGAAGTACGGCCCACCGTGCCCGCCTTGTC |
| | | CCGCGCCGGAGTTCCTCGGCGGTCCCTCGGTCTTTCTGTT |
| | | CCCACCGAAGCCCAAGGACACTTTGATGATTTCCCGCACC |
| | | CCTGAAGTGACATGCGTGGTCGTGGACGTGTCACAGGAAG |
| | | ATCCGGAGGTGCAGTTCAATTGGTACGTGGATGGCGTCGA |
| | | GGTGCACAACGCCAAAACCAAGCCGAGGGAGGAGCAGTT |
| | | CAACTCCACTTACCGCGTCGTGTCCGTGCTGACGGTGCTG |
| | | CATCAGGACTGGCTGAACGGGAAGGAGTACAAGTGCAAAG |
| | | TGTCCAACAAGGGACTTCCTAGCTCAATCGAAAAGACCATC |
| | | TCGAAAGCCAAGGGACAGCCCCGGGAACCCCAAGTGTATA |
| | | CCCTGCCACCGAGCCAGGAAGAAATGACTAAGAACCAAGT |
| | | CTCATTGACTTGCCTTGTGAAGGGCTTCTACCCATCGGATA |
| | | TCGCCGTGGAATGGGAGTCCAACGGCCAGCCGGAAAACA |
| | | ACTACAAGACCACCCCTCCGGTGCTGGACTCAGACGGATC |
| | | CTTCTTCCTCTACTCGCGGCTGACCGTGGATAAGAGCAGA |
| | | TGGCAGGAGGGAAATGTGTTCAGCTGTTCTGTGATGCATG |
| | | AAGCCCTGCACAACCACTACACTCAGAAGTCCCTGTCCCT |
| | | CTCCCTGGGA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 64 | VL | AIQLTQSPSSLSASVGDRVTITCRASESVEYYGTSLMQWYQQ |
| | | KPGKAPKLLIYAASNVESGVPSRFSGSGSGTDFTLTISSLQPE |
| | | DFATYFCQQSRKDPSTFGGGTKVEIK |
| SEQ ID NO: 65 | DNA VL | GCTATTCAGCTGACTCAGTCACCTAGTAGCCTGAGCGCTA |
| | | GTGTGGGCGATAGAGTGACTATCACCTGTAGAGCTAGTGA |
| | | ATCAGTCGAGTACTACGGCACTAGCCTGATGCAGTGGTAT |
| | | CAGCAGAAGCCCGGGAAAGCCCCTAAGCTGCTGATCTACG |
| | | CCGCCTCTAACGTGGAATCAGGCGTGCCCTCTAGGTTTAG |
| | | CGGTAGCGGTAGTGGCACCGACTTCACCCTGACTATCTCT |
| | | AGCCTGCAGCCCGAGGACTTCGCTACCTACTTCTGTCAGC |
| | | AGTCTAGGAAGGACCCTAGCACCTTCGGCGGAGGCACTAA |
| | | GGTCGAGATTAAG |
| SEQ ID NO: 66 | Light | AIQLTQSPSSLSASVGDRVTITCRASESVEYYGTSLMQWYQQ |
| | Chain | KPGKAPKLLIYAASNVESGVPSRFSGSGSGTDFTLTISSLQPE |
| | | DFATYFCQQSRKDPSTFGGGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: 67 | DNA Light | GCTATTCAGCTGACTCAGTCACCTAGTAGCCTGAGCGCTA |
| | Chain | GTGTGGGCGATAGAGTGACTATCACCTGTAGAGCTAGTGA |
| | | ATCAGTCGAGTACTACGGCACTAGCCTGATGCAGTGGTAT |
| | | CAGCAGAAGCCCGGGAAAGCCCCTAAGCTGCTGATCTACG |
| | | CCGCCTCTAACGTGGAATCAGGCGTGCCCTCTAGGTTTAG |
| | | CGGTAGCGGTAGTGGCACCGACTTCACCCTGACTATCTCT |
| | | AGCCTGCAGCCCGAGGACTTCGCTACCTACTTCTGTCAGC |
| | | AGTCTAGGAAGGACCCTAGCACCTTCGGCGGAGGCACTAA |
| | | GGTCGAGATTAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum12 | | |
| SEQ ID NO: 3 | HCDR1 | SYNMH |
| (Kabat) | | |
| SEQ ID NO: 4 | HCDR2 | DIYPGNGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 10 | HCDR2 | YPGNGD |
| (Chothia) | | |
| SEQ ID NO: 5 | HCDR3 | VGGAFPMDY |
| (Chothia) | | |
| SEQ ID NO: 60 | VH | EVQLVQSGAEVKKPGESLKISCKGSGYTFTSYNMHWVRQMP |
| | | GKGLEWMGDIYPGNGDTSYNQKFKGQVTISADKSISTVYLQW |
| | | SSLKASDTAMYYCARVGGAFPMDYWGQGTTVTVSS |
| SEQ ID NO: 61 | DNA VH | GAAGTGCAGCTGGTGCAGTCAGGCGCCGAAGTGAAGAAG |
| | | CCCGGCGAGTCACTGAAGATTAGCTGTAAAGGTTCAGGCT |
| | | ACACCTTCACTAGCTATAATATGCACTGGGTCCGCCAGATG |
| | | CCCGGGAAAGGCCTCGAGTGGATGGGCGATATCTACCCC |
| | | GGGAACGGCGACACTAGTTATAATCAGAAGTTTAAGGGGC |
| | | AAGTCACAATTAGCGCCGATAAGTCTATTAGCACCGTCTAC |
| | | CTGCAGTGGTCTAGCCTGAAGGCTAGTGACACCGCTATGT |
| | | ACTACTGCGCTAGAGTGGGCGGAGCCTTCCCTATGGACTA |
| | | CTGGGGTCAAGGCACTACCGTGACCGTGTCTAGC |
| SEQ ID NO: 62 | Heavy | EVQLVQSGAEVKKPGESLKISCKGSGYTFTSYNMHWVRQMP |
| | Chain | GKGLEWMGDIYPGNGDTSYNQKFKGQVTISADKSISTVYLQW |
| | | SSLKASDTAMYYCARVGGAFPMDYWGQGTTVTVSSASTKGP |
| | | SVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQE |
| | | GNVFSCSVMHEALHNHYTQKSLSLSLG |
| SEQ ID NO: 63 | DNA | GAAGTGCAGCTGGTGCAGTCAGGCGCCGAAGTGAAGAAG |
| | Heavy | CCCGGCGAGTCACTGAAGATTAGCTGTAAAGGTTCAGGCT |
| | Chain | ACACCTTCACTAGCTATAATATGCACTGGGTCCGCCAGATG |
| | | CCCGGGAAAGGCCTCGAGTGGATGGGCGATATCTACCCC |
| | | GGGAACGGCGACACTAGTTATAATCAGAAGTTTAAGGGGC |
| | | AAGTCACAATTAGCGCCGATAAGTCTATTAGCACCGTCTAC |
| | | CTGCAGTGGTCTAGCCTGAAGGCTAGTGACACCGCTATGT |
| | | ACTACTGCGCTAGAGTGGGCGGAGCCTTCCCTATGGACTA |
| | | CTGGGGTCAAGGCACTACCGTGACCGTGTCTAGCGCTAGC |
| | | ACTAAGGGCCCGTCCGTGTTCCCCCTGGCACCTTGTAGCC |
| | | GGAGCACTAGCGAATCCACCGCTGCCCTCGGCTGCCTGGT |
| | | CAAGGATTACTTCCCGGAGCCCGTGACCGTGTCCTGGAAC |
| | | AGCGGAGCCCTGACCTCCGGAGTGCACACCTTCCCCGCT |
| | | GTGCTGCAGAGCTCCGGGCTGTACTCGCTGTCGTCGGTG |
| | | GTCACGGTGCCTTCATCTAGCCTGGGTACCAAGACCTACA |
| | | CTTGCAACGTGGACCACAAGCCTTCCAACACTAAGGTGGA |
| | | CAAGCGCGTCGAATCGAAGTACGGCCCACCGTGCCCGCC |
| | | TTGTCCCGCGCCGGAGTTCCTCGGCGGTCCCTCGGTCTTT |
| | | CTGTTCCCACCGAAGCCCAAGGACACTTTGATGATTTCCCG |
| | | CACCCCTGAAGTGACATGCGTGGTCGTGGACGTGTCACAG |
| | | GAAGATCCGGAGGTGCAGTTCAATTGGTACGTGGATGGCG |
| | | TCGAGGTGCACAACGCCAAAACCAAGCCGAGGGAGGAGC |
| | | AGTTCAACTCCACTTACCGCGTCGTGTCCGTGCTGACGGT |
| | | GCTGCATCAGGACTGGCTGAACGGGAAGGAGTACAAGTGC |
| | | AAAGTGTCCAACAAGGGACTTCCTAGCTCAATCGAAAAGAC |
| | | CATCTCGAAAGCCAAGGGACAGCCCCGGGAACCCCAAGT |
| | | GTATACCCTGCCACCGAGCCAGGAAGAAATGACTAAGAAC |
| | | CAAGTCTCATTGACTTGCCTTGTGAAGGGCTTCTACCCATC |
| | | GGATATCGCCGTGGAATGGGAGTCCAACGGCCAGCCGGA |
| | | AAACAACTACAAGACCACCCCTCCGGTGCTGGACTCAGAC |
| | | GGATCCTTCTTCCTCTACTCGCGGCTGACCGTGGATAAGA |
| | | GCAGATGGCAGGAGGGAAATGTGTTCAGCTGTTCTGTGAT |
| | | GCATGAAGCCCTGCACAACCACTACACTCAGAAGTCCCTG |
| | | TCCCTCTCCCTGGGA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 64 | VL | AIQLTQSPSSLSASBGDRVTITCRASESVEYYGTSLMQWYQQ |
| | | KPGKAPKLLIYAASNVESGVPSRFSGSGSGTDFTLTISSLQPE |
| | | DFATYFCQQSRKDPSTFGGGTKVEIK |
| SEQ ID NO: 65 | DNA VL | GCTATTCAGCTGACTCAGTCACCTAGTAGCCTGAGCGCTA |
| | | GTGTGGGCGATAGAGTGACTATCACCTGTAGAGCTAGTGA |
| | | ATCAGTCGAGTACTACGGCACTAGCCTGATGCAGTGGTAT |
| | | CAGCAGAAGCCCGGGAAAGCCCCTAAGCTGCTGATCTACG |
| | | CCGCCTCTAACGTGGAATCAGGCGTGCCCTCTAGGTTTAG |
| | | CGGTAGCGGTAGTGGCACCGACTTCACCCTGACTATCTCT |
| | | AGCCTGCAGCCCGAGGACTTCGCTACCTACTTCTGTCAGC |
| | | AGTCTAGGAAGGACCCTAGCACCTTCGGCGGAGGCACTAA |
| | | GGTCGAGATTAAG |
| SEQ ID NO: 66 | Light | AIQLTQSPSSLSASVGDRVTITCRASESVEYYGTSLMQWYQQ |
| | Chain | KPGKAPKLLIYAASNVESGVPSRFSGSGSGTDFTLTISSLQPE |
| | | DFATYFCQQSRKDPSTFGGGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: 67 | DNA Light | GCTATTCAGCTGACTCAGTCACCTAGTAGCCTGAGCGCTA |
| | Chain | GTGTGGGCGATAGAGTGACTATCACCTGTAGAGCTAGTGA |
| | | ATCAGTCGAGTACTACGGCACTAGCCTGATGCAGTGGTAT |
| | | CAGCAGAAGCCCGGGAAAGCCCCTAAGCTGCTGATCTACG |
| | | CCGCCTCTAACGTGGAATCAGGCGTGCCCTCTAGGTTTAG |
| | | CGGTAGCGGTAGTGGCACCGACTTCACCCTGACTATCTCT |
| | | AGCCTGCAGCCCGAGGACTTCGCTACCTACTTCTGTCAGC |
| | | AGTCTAGGAAGGACCCTAGCACCTTCGGCGGAGGCACTAA |
| | | GGTCGAGATTAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum13 | | |
| SEQ ID NO: 3 | HCDR1 | SYNMH |
| (Kabat) | | |
| SEQ ID NO: 24 | HCDR2 | DIYPGSGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 25 | HCDR2 | YPGSGD |
| (Chothia) | | |
| SEQ ID NO: 5 | HCDR3 | VGGAFPMDY |
| (Chothia) | | |
| SEQ ID NO: 68 | VH | QVQLVQSGAEVKKPGSSVKVSCKASGYTFTSYNMHWVRQA |
| | | PGQGLEWMGDIYPGSGDTSYNQKFKGRVTITADKSTSTVYM |
| | | ELSSLRSEDTAVYYCARVGGAFPMDYWGQGTTVTVSS |
| SEQ ID NO: 69 | DNA VH | CAGGTGCAATTGGTTCAGTCAGGAGCAGAAGTTAAGAAGC |
| | | CAGGATCATCCGTCAAGGTGTCCTGCAAAGCATCTGGCTA |
| | | CACCTTCACCAGCTACAATATGCACTGGGTCCGACAAGCC |
| | | CCTGGGCAGGGCTTGGAGTGGATGGGAGACATTTACCCC |
| | | GGCAGTGGTGACACTTCCTATAACCAGAAGTTCAAGGGCC |
| | | GAGTCACTATTACCGCTGACAAGTCCACCTCCACAGTCTAC |
| | | ATGGAACTCTCTTCTCTGAGATCCGAGGACACTGCCGTCTA |
| | | TTACTGCGCTCGCGTGGGCGGTGCTTTCCCAATGGACTAT |
| | | TGGGGACAGGGCACAACCGTGACCGTCAGCTCA |
| SEQ ID NO: 70 | Heavy | QVQLVQSGAEVKKPGSSVKVSCKASGYTFTSYNMHWVRQA |
| | Chain | PGQGLEWMGDIYPGSGDTSYNQKFKGRVTITADKSTSTVYM |
| | | ELSSLRSEDTAVYYCARVGGAFPMDYWGQGTTVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPS |
| | | DIAVEWESNGQPENNYKTTPPVLDSDGSFFLYSRLTVDKSRW |
| | | QEGNVFSCSVMHEALHNHYTQKSLSLSLGK |
| SEQ ID NO: 71 | DNA | CAGGTGCAATTGGTTCAGTCAGGAGCAGAAGTTAAGAAGC |
| | Heavy | CAGGATCATCCGTCAAGGTGTCCTGCAAAGCATCTGGCTA |
| | Chain | CACCTTCACCAGCTACAATATGCACTGGGTCCGACAAGCC |
| | | CCTGGGCAGGGCTTGGAGTGGATGGGAGACATTTACCCC |
| | | GGCAGTGGTGACACTTCCTATAACCAGAAGTTCAAGGGCC |
| | | GAGTCACTATTACCGCTGACAAGTCCACCTCCACAGTCTAC |
| | | ATGGAACTCTCTTCTCTGAGATCCGAGGACACTGCCGTCTA |
| | | TTACTGCGCTCGCGTGGGCGGTGCTTTCCCAATGGACTAT |
| | | TGGGGACAGGGCACAACCGTGACCGTCAGCTCAGCCTCTA |
| | | CAAAGGGCCCCTCCGTCTTTCCACTCGCGCCGTGCTCTCG |
| | | CTCCACCTCAGAGTCAACTGCCGCTCTGGGTTGCCTGGTC |
| | | AAGGACTACTTCCCAGAGCCGGTGACAGTGAGCTGGAACA |
| | | GTGGGGCCCTGACATCCGGCGTTCATACCTTCCCCGCAGT |
| | | CCTCCAGTCCTCAGGCCTGTATTCCCTGAGCAGCGTTGTC |
| | | ACAGTGCCCTCCAGCTCTCTTGGCACGAAAACCTACACAT |
| | | GCAACGTTGATCATAAGCCGTCTAATACCAAGGTGGATAAA |
| | | AGAGTGGAGAGCAAGTACGGCCCACCCTGCCCGCCTTGC |
| | | CCAGCTCCGGAGTTCCTGGGCGGACCATCCGTTTTCTTGT |
| | | TTCCACCCAAACCTAAAGACACTCTGATGATTTCCCGAACC |
| | | CCTGAAGTGACTTGCGTTGTGGTGGACGTCTCCCAGGAGG |
| | | ACCCAGAAGTGCAATTCAACTGGTACGTGGACGGGGTGGA |
| | | GGTGCACAATGCAAAAACCAAACCAAGGGAGGAACAGTTT |
| | | AATTCAACATATAGGGTTGTGTCTGTGCTGACGGTTCTGCA |
| | | TCAGGACTGGCTGAACGGAAAGGAATACAAGTGCAAGGTG |
| | | TCCAACAAAGGACTGCCAAGCTCTATCGAGAAAACAATCTC |
| | | TAAGGCCAAGGGACAACCTAGAGAGCCCCAAGTTTACACC |
| | | CTGCCACCATCACAGGAAGAGATGACCAAAAATCAGGTGA |
| | | GCTTGACATGCCTGGTGAAGGGCTTCTACCCTAGCGATATT |
| | | GCGGTTGAGTGGGAGTCAAATGGCCAGCCTGAGAACAACT |
| | | ATAAGACTACTCCTCCCGTGCTGGACTCCGACGGGAGCTT |
| | | TTTCCTGTATTCCAGGCTTACAGTCGATAAGAGCAGATGGC |
| | | AAGAGGGGAATGTGTTTTCCTGCTCCGTGATGCACGAGGC |
| | | TCTCCATAACCATTATACTCAGAAAAGTCTCTCTCTGTCACT |
| | | GGGCAAA |
| SEQ ID NO: 6 | LDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 64 | VL | AIQLTQSPSSLSASVGDRVTITCRASESVEYYGTSLMQWYQQ |
| | | KPGKAPKLLIYAASNVESGVPSRFSGSGSGTDFTLTISSLQPE |
| | | DFATYFCQQSRKDPSTFGGGTKVEIK |
| SEQ ID NO: | DNA VL | GCAATACAGTTGACACAGAGTCCTTCAAGTTTGTCCGCTTC |
| 125 | | CGTTGGCGACCGAGTGACAATCACCTGTAGAGCATCCGAG |
| | | TCAGTGGAGTATTATGGCACTAGCCTGATGCAGTGGTATCA |
| | | GCAAAAGCCAGGGAAAGCCCCAAAGCTGCTGATATATGCC |
| | | GCGAGTAACGTCGAGTCAGGGGTGCCATCAAGATTCTCCG |
| | | GTTCCGGGTCCGGAACCGACTTCACACTGACCATCTCTTC |
| | | CCTTCAGCCAGAGGACTTCGCTACGTACTTTTGCCAGCAGT |
| | | CACGGAAAGATCCCTCTACTTTCGGAGGTGGGACAAAAGT |
| | | CGAAATTAAA |
| SEQ ID NO: 66 | Light | AIQLTQSPSSLSASVGDRVTITCRASESVEYYGTSLMQWYQQ |
| | Chain | KPGKAPKLLIYAASNVESGVPSRFSGSGSGTDFTLTISSLQPE |
| | | DFATYFCQQSRKDPSTFGGGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: | DNA Light | GCAATACAGTTGACACAGAGTCCTTCAAGTTTGTCCGCTTC |
| 126 | Chain | CGTTGGCGACCGAGTGACAATCACCTGTAGAGCATCCGAG |
| | | TCAGTGGAGTATTATGGCACTAGCCTGATGCAGTGGTATCA |
| | | GCAAAAGCCAGGGAAAGCCCCAAAGCTGCTGATATATGCC |
| | | GCGAGTAACGTCGAGTCAGGGGTGCCATCAAGATTCTCCG |
| | | GTTCCGGGTCCGGAACCGACTTCACACTGACCATCTCTTC |
| | | CCTTCAGCCAGAGGACTTCGCTACGTACTTTTGCCAGCAGT |
| | | CACGGAAAGATCCCTCTACTTTCGGAGGTGGGACAAAAGT |
| | | CGAAATTAAACGTACGGTGGCAGCTCCGTCTGTTTTCATCT |
| | | TTCCACCTAGCGACGAGCAACTCAAAAGTGGTACAGCATC |
| | | CGTGGTTTGTCTGCTGAACAATTTTTACCCCAGGGAGGCTA |
| | | AGGTCCAGTGGAAAGTCGATAACGCTCTTCAGTCTGGCAA |
| | | CAGTCAGGAGAGCGTCACAGAGCAGGACTCTAAGGATAGC |
| | | ACTTATAGTCTGTCCTCCACGCTGACACTGTCTAAAGCGGA |
| | | TTATGAGAAGCACAAGGTTTACGCCTGTGAGGTAACGCAC |
| | | CAAGGACTCTCCTCCCCAGTTACCAAATCTTTCAACAGAGG |
| | | AGAATGT |
| ABTIM3- | | |
| hum14 | | |
| SEQ ID NO: 3 | HCDR1 | SYNMH |
| (Kabat) | | |
| SEQ ID NO: 30 | HCDR2 | DIYPGQGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 31 | HCDR2 | YPGQGD |
| (Chothia) | | |
| SEQ ID NO: 5 | HCDR3 | VGGAFPMDY |
| (Chothia) | | |
| SEQ ID NO: 72 | VH | QVQLVQSGAEVKKPGSSVKVSCKASGYTFTSYNMHWVRQA |
| | | PGQGLEWMGDIYPGQGDTSYNQKFKGRVTITADKSTSTVYM |
| | | ELSSLRSEDTAVYYCARVGGAFPMDYWGQGTTVTVSS |
| SEQ ID NO: 73 | DNA VH | CAGGTGCAATTGGTTCAGTCAGGAGCAGAAGTTAAGAAGC |
| | | CAGGATCATCCGTCAAGGTGTCCTGCAAAGCATCTGGCTA |
| | | CACCTTCACCAGCTACAATATGCACTGGGTCCGACAAGCC |
| | | CCTGGGCAGGGCTTGGAGTGGATGGGAGACATTTACCCC |
| | | GGCCAGGGTGACACTTCCTATAACCAGAAGTTCAAGGGCC |
| | | GAGTCACTATTACCGCTGACAAGTCCACCTCCACAGTCTAC |
| | | ATGGAACTCTCTTCTCTGAGATCCGAGGACACTGCCGTCTA |
| | | TTACTGCGCTCGCGTGGGCGGTGCTTTCCCAATGGACTAT |
| | | TGGGGACAGGGCACAACCGTGACCGTCAGCTCA |
| SEQ ID NO: 74 | Heavy | QVQLVQSGAEVKKPGSSVKVSCKASGYTFTSYNMHWVRQA |
| | Chain | PGQGLEWMGDIYPGQGDTSYNQKFKGRVTITADKSTSTVYM |
| | | ELSSLRSEDTAVYYCARVGGAFPMDYWGQGTTVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPS |
| | | DIAVEWESNGQPENNYKTTPPVLDSDGSFFLYSRLTVDKSRW |
| | | QEGNVFSCSVMHEALHNHYTQKSLSLSLGK |
| SEQ ID NO: 75 | DNA | CAGGTGCAATTGGTTCAGTCAGGAGCAGAAGTTAAGAAGC |
| | Heavy | CAGGATCATCCGTCAAGGTGTCCTGCAAAGCATCTGGCTA |
| | Chain | CACCTTCACCAGCTACAATATGCACTGGGTCCGACAAGCC |
| | | CCTGGGCAGGGCTTGGAGTGGATGGGAGACATTTACCCC |
| | | GGCCAGGGTGACACTTCCTATAACCAGAAGTTCAAGGGCC |
| | | GAGTCACTATTACCGCTGACAAGTCCACCTCCACAGTCTAC |
| | | ATGGAACTCTCTTCTCTGAGATCCGAGGACACTGCCGTCTA |
| | | TTACTGCGCTCGCGTGGGCGGTGCTTTCCCAATGGACTAT |
| | | TGGGGACAGGGCACAACCGTGACCGTCAGCTCAGCCTCTA |
| | | CAAAGGGCCCCTCCGTCTTTCCACTCGCGCCGTGCTCTCG |
| | | CTCCACCTCAGAGTCAACTGCCGCTCTGGGTTGCCTGGTC |
| | | AAGGACTACTTCCCAGAGCCGGTGACAGTGAGCTGGAACA |
| | | GTGGGGCCCTGACATCCGGCGTTCATACCTTCCCCGCAGT |
| | | CCTCCAGTCCTCAGGCCTGTATTCCCTGAGCAGCGTTGTC |
| | | ACAGTGCCCTCCAGCTCTCTTGGCACGAAAACCTACACAT |
| | | GCAACGTTGATCATAAGCCGTCTAATACCAAGGTGGATAAA |
| | | AGAGTGGAGAGCAAGTACGGCCCACCCTGCCCGCCTTGC |
| | | CCAGCTCCGGAGTTCCTGGGCGGACCATCCGTTTTCTTGT |
| | | TTCCACCCAAACCTAAAGACACTCTGATGATTTCCCGAACC |
| | | CCTGAAGTGACTTGCGTTGTGGTGGACGTCTCCCAGGAGG |
| | | ACCCAGAAGTGCAATTCAACTGGTACGTGGACGGGGTGGA |
| | | GGTGCACAATGCAAAAACCAAACCAAGGGAGGAACAGTTT |
| | | AATTCAACATATAGGGTTGTGTCTGTGCTGACGGTTCTGCA |
| | | TCAGGACTGGCTGAACGGAAAGGAATACAAGTGCAAGGTG |
| | | TCCAACAAAGGACTGCCAAGCTCTATCGAGAAAACAATCTC |
| | | TAAGGCCAAGGGACAACCTAGAGAGCCCCAAGTTTACACC |
| | | CTGCCACCATCACAGGAAGAGATGACCAAAAATCAGGTGA |
| | | GCTTGACATGCCTGGTGAAGGGCTTCTACCCTAGCGATATT |
| | | GCGGTTGAGTGGGAGTCAAATGGCCAGCCTGAGAACAACT |
| | | ATAAGACTACTCCTCCCGTGCTGGACTCCGACGGGAGCTT |
| | | TTTCCTGTATTCCAGGCTTACAGTCGATAAGAGCAGATGGC |
| | | AAGAGGGGAATGTGTTTTCCTGCTCCGTGATGCACGAGGC |
| | | TCTCCATAACCATTATACTCAGAAAAGTCTCTCTCTGTCACT |
| | | GGGCAAA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTS; |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 64 | VL | AIQLTQSPSSLSASVGDRVTITCRASESVEYYGTSLMQWYQQ |
| | | KPGKAPKLLIYAASNVESGVPSRFSGSGSGTDFTLTISSLQPE |
| | | DFATYFCQQSRKDPSTFGGGTKVEIK |
| SEQID NO: | DNA VL | GCAATACAGTTGACACAGAGTCCTTCAAGTTTGTCCGCTTC |
| 125 | | CGTTGGCGACCGAGTGACAATCACCTGTAGAGCATCCGAG |
| | | TCAGTGGAGTATTATGGCACTAGCCTGATGCAGTGGTATCA |
| | | GCAAAAGCCAGGGAAAGCCCCAAAGCTGCTGATATATGCC |
| | | GCGAGTAACGTCGAGTCAGGGGTGCCATCAAGATTCTCCG |
| | | GTTCCGGGTCCGGAACCGACTTCACACTGACCATCTCTTC |
| | | CCTTCAGCCAGAGGACTTCGCTACGTACTTTTGCCAGCAGT |
| | | CACGGAAAGATCCCTCTACTTTCGGAGGTGGGACAAAAGT |
| | | CGAAATTAAA |
| SEQ ID NO: 66 | Light | AIQLTQSPSSLSASBGDRVTITCRASESVEYYGTSLMQWYQQ |
| | Chain | KPGKAPKLLIYAASNVESGVPSRFSGSGSGTDFTLTISSLQPE |
| | | DFATYFCQQSRKDPSTFGGGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: | DNA Light | GCAATACAGTTGACACAGAGTCCTTCAAGTTTGTCCGCTTC |
| 126 | Chain | CGTTGGCGACCGAGTGACAATCACCTGTAGAGCATCCGAG |
| | | TCAGTGGAGTATTATGGCACTAGCCTGATGCAGTGGTATCA |
| | | GCAAAAGCCAGGGAAAGCCCCAAAGCTGCTGATATATGCC |
| | | GCGAGTAACGTCGAGTCAGGGGTGCCATCAAGATTCTCCG |
| | | GTTCCGGGTCCGGAACCGACTTCACACTGACCATCTCTTC |
| | | CCTTCAGCCAGAGGACTTCGCTACGTACTTTTGCCAGCAGT |
| | | CACGGAAAGATCCCTCTACTTTCGGAGGTGGGACAAAAGT |
| | | CGAAATTAAACGTACGGTGGCAGCTCCGTCTGTTTTCATCT |
| | | TTCCACCTAGCGACGAGCAACTCAAAAGTGGTACAGCATC |
| | | CGTGGTTTGTCTGCTGAACAATTTTTACCCCAGGGAGGCTA |
| | | AGGTCCAGTGGAAAGTCGATAACGCTCTTCAGTCTGGCAA |
| | | CAGTCAGGAGAGCGTCACAGAGCAGGACTCTAAGGATAGC |
| | | ACTTATAGTCTGTCCTCCACGCTGACACTGTCTAAAGCGGA |
| | | TTATGAGAAGCACAAGGTTTACGCCTGTGAGGTAACGCAC |
| | | CAAGGACTCTCCTCCCCAGTTACCAAATCTTTCAACAGAGG |
| | | AGAATGT |
| ABTIM3- | | |
| hum15 | | |
| SEQ ID NO: 3 | HCDR1 | SYNMH |
| (Kabat) | | |
| SEQ ID NO: 24 | HCDR2 | DIYPGSGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 25 | HCDR2 | YPGSGD |
| (Chothia) | | |
| SEQ ID NO: 5 | HCDR3 | VGGAFPMDY |
| (Chothia) | | |
| SEQ ID NO: 76 | VH | EVQLVQSGAEVKKPGESLKISCKGSGYTFTSYNMHWVRQMP |
| | | GKGLEWMGDIYPGSGDTSYNQKFKGQVTISADKSISTVYLQW |
| | | SSLKASDTAMYYCARVGGAFPMDYWGQGTTVTVSS |
| SEQ ID NO: 77 | DNA VH | GAAGTTCAATTGGTACAGTCTGGCGCAGAAGTAAAGAAAC |
| | | CAGGAGAGAGTTTGAAAATTTCCTGCAAGGGCAGTGGGTA |
| | | CACATTCACGTCCTACAATATGCACTGGGTGAGACAGATGC |
| | | CAGGCAAGGGCCTGGAGTGGATGGGAGACATATACCCAG |
| | | GCAGTGGAGACACAAGCTATAATCAGAAATTCAAAGGACA |
| | | GGTGACGATCTCCGCAGACAAATCCATATCTACGGTCTACC |
| | | TCCAGTGGTCCTCACTTAAAGCCTCCGACACCGCCATGTA |
| | | CTATTGCGCTCGGGTAGGTGGCGCGTTTCCAATGGACTAT |
| | | TGGGGCCAAGGGACCACAGTAACCGTCAGCTCA |
| SEQ ID NO: 78 | Heavy | EVQLVQSGAEVKKPGESLKISCKGSGYTFTSYNMHWVRQMP |
| | Chain | GKGLEWMGDIYPGSGDTSYNQKFKGQVTISADKSISTVYLQW |
| | | SSLKASDTAMYYCARVGGAFPMDYWGQGTTVTVSSASTKGP |
| | | SVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQE |
| | | GNVFSCSVMHEALHNHYTQKSLSLSLGK |
| SEQ ID NO: 79 | DNA | GAAGTTCAATTGGTACAGTCTGGCGCAGAAGTAAAGAAAC |
| | Heavy | CAGGAGAGAGTTTGAAAATTTCCTGCAAGGGCAGTGGGTA |
| | Chain | CACATTCACGTCCTACAATATGCACTGGGTGAGACAGATGC |
| | | CAGGCAAGGGCCTGGAGTGGATGGGAGACATATACCCAG |
| | | GCAGTGGAGACACAAGCTATAATCAGAAATTCAAAGGACA |
| | | GGTGACGATCTCCGCAGACAAATCCATATCTACGGTCTACC |
| | | TCCAGTGGTCCTCACTTAAAGCCTCCGACACCGCCATGTA |
| | | CTATTGCGCTCGGGTAGGTGGCGCGTTTCCAATGGACTAT |
| | | TGGGGCCAAGGGACCACAGTAACCGTCAGCTCAGCCTCTA |
| | | CAAAGGGCCCCTCCGTCTTTCCACTCGCGCCGTGCTCTCG |
| | | CTCCACCTCAGAGTCAACTGCCGCTCTGGGTTGCCTGGTC |
| | | AAGGACTACTTCCCAGAGCCGGTGACAGTGAGCTGGAACA |
| | | GTGGGGCCCTGACATCCGGCGTTCATACCTTCCCCGCAGT |
| | | CCTCCAGTCCTCAGGCCTGTATTCCCTGAGCAGCGTTGTC |
| | | ACAGTGCCCTCCAGCTCTCTTGGCACGAAAACCTACACAT |
| | | GCAACGTTGATCATAAGCCGTCTAATACCAAGGTGGATAAA |
| | | AGAGTGGAGAGCAAGTACGGCCCACCCTGCCCGCCTTGC |
| | | CCAGCTCCGGAGTTCCTGGGCGGACCATCCGTTTTCTTGT |
| | | TTCCACCCAAACCTAAAGACACTCTGATGATTTCCCGAACC |
| | | CCTGAAGTGACTTGCGTTGTGGTGGACGTCTCCCAGGAGG |
| | | ACCCAGAAGTGCAATTCAACTGGTACGTGGACGGGGTGGA |
| | | GGTGCACAATGCAAAAACCAAACCAAGGGAGGAACAGTTT |
| | | AATTCAACATATAGGGTTGTGTCTGTGCTGACGGTTCTGCA |
| | | TCAGGACTGGCTGAACGGAAAGGAATACAAGTGCAAGGTG |
| | | TCCAACAAAGGACTGCCAAGCTCTATCGAGAAAACAATCTC |
| | | TAAGGCCAAGGGACAACCTAGAGAGCCCCAAGTTTACACC |
| | | CTGCCACCATCACAGGAAGAGATGACCAAAAATCAGGTGA |
| | | GCTTGACATGCCTGGTGAAGGGCTTCTACCCTAGCGATATT |
| | | GCGGTTGAGTGGGAGTCAAATGGCCAGCCTGAGAACAACT |
| | | ATAAGACTACTCCTCCCGTGCTGGACTCCGACGGGAGCTT |
| | | TTTCCTGTATTCCAGGCTTACAGTCGATAAGAGCAGATGGC |
| | | AAGAGGGGAATGTGTTTTCCTGCTCCGTGATGCACGAGGC |
| | | TCTCCATAACCATTATACTCAGAAAAGTCTCTCTCTGTCACT |
| | | GGGCAAA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 56 | VL | EIVLTQSPATLSLSPGERATLSCRASESVEYYGTSLMQWYQQ |
| | | KPGQAPRLLIYAASNVESGIPARFSGSGSGTDFTLTISSLEPED |
| | | IAVYFCQQSRKDPSTFGGGTKVEIK |
| SEQ ID NO: | DNA VL | GAGATTGTTCTTACGCAAAGTCCCGCCACACTTAGTTTGTC |
| 127 | | ACCAGGAGAGCGCGCCACCCTGAGCTGCAGAGCTTCAGA |
| | | GAGTGTGGAATACTACGGCACATCCCTGATGCAGTGGTAT |
| | | CAGCAGAAACCAGGACAGGCTCCTCGGCTGCTGATCTACG |
| | | CAGCCAGCAACGTCGAGTCCGGCATTCCAGCCAGATTTTC |
| | | TGGGTCAGGATCTGGAACTGACTTTACACTGACAATCTCCA |
| | | GCCTGGAACCCGAGGACATTGCTGTGTATTTTTGTCAACAG |
| | | TCCCGGAAGGACCCCAGTACCTTTGGAGGTGGAACCAAGG |
| | | TAGAGATAAAG |
| SEQ ID NO: 58 | Light | EIVLTQSPATLSLSPGERATLSCRASESVEYYGTSLMQWYQQ |
| | Chain | KPGQAPRLLIYAASNVESGIPARFSGSGSGTDFTLTISSLEPED |
| | | IAVYFCQQSRKDPSTFGGGTKVEIKRTVAAPSVFIFPPSDEQL |
| | | KSGTASVVCLLNNFYPREAKVQWKVDNALQSGNSQESVTEQ |
| | | DSKDSTYSLSSTLTLSKADYEKHKVYACEVTHQGLSSPVTKSF |
| | | NRGEC |
| SEQ ID NO: | DNA Light | GAGATTGTTCTTACGCAAAGTCCCGCCACACTTAGTTTGTC |
| 128 | Chain | ACCAGGAGAGCGCGCCACCCTGAGCTGCAGAGCTTCAGA |
| | | GAGTGTGGAATACTACGGCACATCCCTGATGCAGTGGTAT |
| | | CAGCAGAAACCAGGACAGGCTCCTCGGCTGCTGATCTACG |
| | | CAGCCAGCAACGTCGAGTCCGGCATTCCAGCCAGATTTTC |
| | | TGGGTCAGGATCTGGAACTGACTTTACACTGACAATCTCCA |
| | | GCCTGGAACCCGAGGACATTGCTGTGTATTTTTGTCAACAG |
| | | TCCCGGAAGGACCCCAGTACCTTTGGAGGTGGAACCAAGG |
| | | TAGAGATAAAGCGTACGGTGGCAGCTCCGTCTGTTTTCATC |
| | | TTTCCACCTAGCGACGAGCAACTCAAAAGTGGTACAGCATC |
| | | CGTGGTTTGTCTGCTGAACAATTTTTACCCCAGGGAGGCTA |
| | | AGGTCCAGTGGAAAGTCGATAACGCTCTTCAGTCTGGCAA |
| | | CAGTCAGGAGAGCGTCACAGAGCAGGACTCTAAGGATAGC |
| | | ACTTATAGTCTGTCCTCCACGCTGACACTGTCTAAAGCGGA |
| | | TTATGAGAAGCACAAGGTTTACGCCTGTGAGGTAACGCAC |
| | | CAAGGACTCTCCTCCCCAGTTACCAAATCTTTCAACAGAGG |
| | | AGAATGT |
| ABTIM3- | | |
| hum16 | | |
| SEQ ID NO: 3 | HCDR1 | SYNMH |
| (Kabat) | | |
| SEQ ID NO: 30 | HCDR2 | DIYPGQGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 31 | HCDR2 | YPGQGD |
| (Chothia) | | |
| SEQ ID NO: 5 | HCDR3 | VGGAFPMDY |
| (Chothia) | | |
| SEQ ID NO: 80 | VH | EVQLVQSGAEVKKPGESLKISCKGSGYTFTSYNMHWVRQMP |
| | | GKGLEWMGDIYPGQGDTSYNQKFKGQVTISADKSISTVYLQW |
| | | SSLKASDTAMYYCARVGGAFPMDYWGQGTTVTVSS |
| SEQ ID NO: 81 | DNA VH | GAAGTTCAATTGGTACAGTCTGGCGCAGAAGTAAAGAAAC |
| | | CAGGAGAGAGTTTGAAAATTTCCTGCAAGGGCAGTGGGTA |
| | | CACATTCACGTCCTACAATATGCACTGGGTGAGACAGATGC |
| | | CAGGCAAGGGCCTGGAGTGGATGGGAGACATATACCCAG |
| | | GCCAGGGAGACACAAGCTATAATCAGAAATTCAAAGGACA |
| | | GGTGACGATCTCCGCAGACAAATCCATATCTACGGTCTACC |
| | | TCCAGTGGTCCTCACTTAAAGCCTCCGACACCGCCATGTA |
| | | CTATTGCGCTCGGGTAGGTGGCGCGTTTCCAATGGACTAT |
| | | TGGGGCCAAGGGACCACAGTAACCGTCAGCTCA |
| SEQ ID NO: 82 | Heavy | EVQLVQSGAEVKKPGESLKISCKGSGYTFTSYNMHWVRQMP |
| | Chain | GKGLEWMGDIYPGQGDTSYNQKFKGQVTISADKSISTVYLQW |
| | | SSLKASDTAMYYCARVGGAFPMDYWGQGTTVTVSSASTKGP |
| | | SVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQE |
| | | GNVFSCSVMHEALHNHYTQKSLSLSLGK |
| SEQ ID NO: 83 | DNA | GAAGTTCAATTGGTACAGTCTGGCGCAGAAGTAAAGAAAC |
| | Heavy | CAGGAGAGAGTTTGAAAATTTCCTGCAAGGGCAGTGGGTA |
| | Chain | CACATTCACGTCCTACAATATGCACTGGGTGAGACAGATGC |
| | | CAGGCAAGGGCCTGGAGTGGATGGGAGACATATACCCAG |
| | | GCCAGGGAGACACAAGCTATAATCAGAAATTCAAAGGACA |
| | | GGTGACGATCTCCGCAGACAAATCCATATCTACGGTCTACC |
| | | TCCAGTGGTCCTCACTTAAAGCCTCCGACACCGCCATGTA |
| | | CTATTGCGCTCGGGTAGGTGGCGCGTTTCCAATGGACTAT |
| | | TGGGGCCAAGGGACCACAGTAACCGTCAGCTCAGCCTCTA |
| | | CAAAGGGCCCCTCCGTCTTTCCACTCGCGCCGTGCTCTCG |
| | | CTCCACCTCAGAGTCAACTGCCGCTCTGGGTTGCCTGGTC |
| | | AAGGACTACTTCCCAGAGCCGGTGACAGTGAGCTGGAACA |
| | | GTGGGGCCCTGACATCCGGCGTTCATACCTTCCCCGCAGT |
| | | CCTCCAGTCCTCAGGCCTGTATTCCCTGAGCAGCGTTGTC |
| | | ACAGTGCCCTCCAGCTCTCTTGGCACGAAAACCTACACAT |
| | | GCAACGTTGATCATAAGCCGTCTAATACCAAGGTGGATAAA |
| | | AGAGTGGAGAGCAAGTACGGCCCACCCTGCCCGCCTTGC |
| | | CCAGCTCCGGAGTTCCTGGGCGGACCATCCGTTTTCTTGT |
| | | TTCCACCCAAACCTAAAGACACTCTGATGATTTCCCGAACC |
| | | CCTGAAGTGACTTGCGTTGTGGTGGACGTCTCCCAGGAGG |
| | | ACCCAGAAGTGCAATTCAACTGGTACGTGGACGGGGTGGA |
| | | GGTGCACAATGCAAAAACCAAACCAAGGGAGGAACAGTTT |
| | | AATTCAACATATAGGGTTGTGTCTGTGCTGACGGTTCTGCA |
| | | TCAGGACTGGCTGAACGGAAAGGAATACAAGTGCAAGGTG |
| | | TCCAACAAAGGACTGCCAAGCTCTATCGAGAAAACAATCTC |
| | | TAAGGCCAAGGGACAACCTAGAGAGCCCCAAGTTTACACC |
| | | CTGCCACCATCACAGGAAGAGATGACCAAAAATCAGGTGA |
| | | GCTTGACATGCCTGGTGAAGGGCTTCTACCCTAGCGATATT |
| | | GCGGTTGAGTGGGAGTCAAATGGCCAGCCTGAGAACAACT |
| | | ATAAGACTACTCCTCCCGTGCTGGACTCCGACGGGAGCTT |
| | | TTTCCTGTATTCCAGGCTTACAGTCGATAAGAGCAGATGGC |
| | | AAGAGGGGAATGTGTTTTCCTGCTCCGTGATGCACGAGGC |
| | | TCTCCATAACCATTATACTCAGAAAAGTCTCTCTCTGTCACT |
| | | GGGCAAA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 56 | VL | EIVLTQSPATLSLSPGERATLSCRASESVEYYGTSLMQWYQQ |
| | | KPGQAPRLLIYAASNVESGIPARFSGSGSGTDFTLTISSLEPED |
| | | IAVYFCQQSRKDPSTFGGGTKVEIK |
| SEQ ID NO: | DNA VL | GAGATTGTTCTTACGCAAAGTCCCGCCACACTTAGTTTGTC |
| 127 | | ACCAGGAGAGCGCGCCACCCTGAGCTGCAGAGCTTCAGA |
| | | GAGTGTGGAATACTACGGCACATCCCTGATGCAGTGGTAT |
| | | CAGCAGAAACCAGGACAGGCTCCTCGGCTGCTGATCTACG |
| | | CAGCCAGCAACGTCGAGTCCGGCATTCCAGCCAGATTTTC |
| | | TGGGTCAGGATCTGGAACTGACTTTACACTGACAATCTCCA |
| | | GCCTGGAACCCGAGGACATTGCTGTGTATTTTTGTCAACAG |
| | | TCCCGGAAGGACCCCAGTACCTTTGGAGGTGGAACCAAGG |
| | | TAGAGATAAAG |
| SEQ ID NO: 58 | Light | EIVLTQSPATLSLSPGERATLSCRASESVEYYGTSLMQWYQQ |
| | Chain | KPGQAPRLLIYAASNVESGIPARFSGSGSGTDFTLTISSLEPED |
| | | IAVYFCQQSRKDPSTFGGGTKVEIKRTVAAPSVFIFPPSDEQL |
| | | KSGTASVVCLLNNFYPREAKVQWKVDNALQSGNSQESVTEQ |
| | | DSKDSTYSLSSTLTLSKADYEKHKVYACEVTHQGLSSPVTKSF |
| | | NRGEC |
| SEQ ID NO: | DNA Light | GAGATTGTTCTTACGCAAAGTCCCGCCACACTTAGTTTGTC |
| 128 | Chain | ACCAGGAGAGCGCGCCACCCTGAGCTGCAGAGCTTCAGA |
| | | GAGTGTGGAATACTACGGCACATCCCTGATGCAGTGGTAT |
| | | CAGCAGAAACCAGGACAGGCTCCTCGGCTGCTGATCTACG |
| | | CAGCCAGCAACGTCGAGTCCGGCATTCCAGCCAGATTTTC |
| | | TGGGTCAGGATCTGGAACTGACTTTACACTGACAATCTCCA |
| | | GCCTGGAACCCGAGGACATTGCTGTGTATTTTTGTCAACAG |
| | | TCCCGGAAGGACCCCAGTACCTTTGGAGGTGGAACCAAGG |
| | | TAGAGATAAAGCGTACGGTGGCAGCTCCGTCTGTTTTCATC |
| | | TTTCCACCTAGCGACGAGCAACTCAAAAGTGGTACAGCATC |
| | | CGTGGTTTGTCTGCTGAACAATTTTTACCCCAGGGAGGCTA |
| | | AGGTCCAGTGGAAAGTCGATAACGCTCTTCAGTCTGGCAA |
| | | CAGTCAGGAGAGCGTCACAGAGCAGGACTCTAAGGATAGC |
| | | ACTTATAGTCTGTCCTCCACGCTGACACTGTCTAAAGCGGA |
| | | TTATGAGAAGCACAAGGTTTACGCCTGTGAGGTAACGCAC |
| | | CAAGGACTCTCCTCCCCAGTTACCAAATCTTTCAACAGAGG |
| | | AGAATGT |
| ABTIM3- | | |
| hum17 | | |
| SEQ ID NO: 3 | HCDR1 | SYNMH |
| (Kabat) | | |
| SEQ ID NO: 24 | HCDR2 | DIYPGSGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 25 | HCDR2 | YPGSGD |
| (Chothia) | | |
| SEQ ID NO: 5 | HCDR3 | VGGAFPMDY |
| (Chothia) | | |
| SEQ ID NO: 68 | VH | QVQLVQSGAEVKKPGSSVKVSCKASGYTFTSYNMHWVRQA |
| | | PGQGLEWMGDIYPGSGDTSYNQKFKGRVTITADKSTSTVYM |
| | | ELSSLRSEDTAVYYCARVGGAFPMDYWGQGTTVTVSS |
| SEQ ID NO: 69 | DNA VH | CAGGTGCAATTGGTTCAGTCAGGAGCAGAAGTTAAGAAGC |
| | | CAGGATCATCCGTCAAGGTGTCCTGCAAAGCATCTGGCTA |
| | | CACCTTCACCAGCTACAATATGCACTGGGTCCGACAAGCC |
| | | CCTGGGCAGGGCTTGGAGTGGATGGGAGACATTTACCCC |
| | | GGCAGTGGTGACACTTCCTATAACCAGAAGTTCAAGGGCC |
| | | GAGTCACTATTACCGCTGACAAGTCCACCTCCACAGTCTAC |
| | | ATGGAACTCTCTTCTCTGAGATCCGAGGACACTGCCGTCTA |
| | | TTACTGCGCTCGCGTGGGCGGTGCTTTCCCAATGGACTAT |
| | | TGGGGACAGGGCACAACCGTGACCGTCAGCTCA |
| SEQ ID NO: 70 | Heavy | QVQLVQSGAEVKKPGSSVKVSCKASGYTFTSYNMHWVRQA |
| | Chain | PGQGLEWMGDIYPGSGDTSYNQKFKGRVTITADKSTSTVYM |
| | | ELSSLRSEDTAVYYCARVGGAFPMDYWGQGTTVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPS |
| | | DIAVEWESNGQPENNYKTTPPVLDSDGSFFLYSRLTVDKSRW |
| | | QEGNVFSCSVMHEALHNHYTQKSLSLSLGK |
| SEQ ID NO: 71 | DNA | CAGGTGCAATTGGTTCAGTCAGGAGCAGAAGTTAAGAAGC |
| | Heavy | CAGGATCATCCGTCAAGGTGTCCTGCAAAGCATCTGGCTA |
| | Chain | CACCTTCACCAGCTACAATATGCACTGGGTCCGACAAGCC |
| | | CCTGGGCAGGGCTTGGAGTGGATGGGAGACATTTACCCC |
| | | GGCAGTGGTGACACTTCCTATAACCAGAAGTTCAAGGGCC |
| | | GAGTCACTATTACCGCTGACAAGTCCACCTCCACAGTCTAC |
| | | ATGGAACTCTCTTCTCTGAGATCCGAGGACACTGCCGTCTA |
| | | TTACTGCGCTCGCGTGGGCGGTGCTTTCCCAATGGACTAT |
| | | TGGGGACAGGGCACAACCGTGACCGTCAGCTCAGCCTCTA |
| | | CAAAGGGCCCCTCCGTCTTTCCACTCGCGCCGTGCTCTCG |
| | | CTCCACCTCAGAGTCAACTGCCGCTCTGGGTTGCCTGGTC |
| | | AAGGACTACTTCCCAGAGCCGGTGACAGTGAGCTGGAACA |
| | | GTGGGGCCCTGACATCCGGCGTTCATACCTTCCCCGCAGT |
| | | CCTCCAGTCCTCAGGCCTGTATTCCCTGAGCAGCGTTGTC |
| | | ACAGTGCCCTCCAGCTCTCTTGGCACGAAAACCTACACAT |
| | | GCAACGTTGATCATAAGCCGTCTAATACCAAGGTGGATAAA |
| | | AGAGTGGAGAGCAAGTACGGCCCACCCTGCCCGCCTTGC |
| | | CCAGCTCCGGAGTTCCTGGGCGGACCATCCGTTTTCTTGT |
| | | TTCCACCCAAACCTAAAGACACTCTGATGATTTCCCGAACC |
| | | CCTGAAGTGACTTGCGTTGTGGTGGACGTCTCCCAGGAGG |
| | | ACCCAGAAGTGCAATTCAACTGGTACGTGGACGGGGTGGA |
| | | GGTGCACAATGCAAAAACCAAACCAAGGGAGGAACAGTTT |
| | | AATTCAACATATAGGGTTGTGTCTGTGCTGACGGTTCTGCA |
| | | TCAGGACTGGCTGAACGGAAAGGAATACAAGTGCAAGGTG |
| | | TCCAACAAAGGACTGCCAAGCTCTATCGAGAAAACAATCTC |
| | | TAAGGCCAAGGGACAACCTAGAGAGCCCCAAGTTTACACC |
| | | CTGCCACCATCACAGGAAGAGATGACCAAAAATCAGGTGA |
| | | GCTTGACATGCCTGGTGAAGGGCTTCTACCCTAGCGATATT |
| | | GCGGTTGAGTGGGAGTCAAATGGCCAGCCTGAGAACAACT |
| | | ATAAGACTACTCCTCCCGTGCTGGACTCCGACGGGAGCTT |
| | | TTTCCTGTATTCCAGGCTTACAGTCGATAAGAGCAGATGGC |
| | | AAGAGGGGAATGTGTTTTCCTGCTCCGTGATGCACGAGGC |
| | | TCTCCATAACCATTATACTCAGAAAAGTCTCTCTCTGTCACT |
| | | GGGCAAA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 56 | VL | EIVLTQSPATLSLSPGERATLSCRASESVEYYGTSLMQWYQQ |
| | | KPGQAPRLLIYAASNVESGIPARFSGSGSGTDFTLTISSLEPED |
| | | IAVYFCQQSRKDPSTFGGGTKVEIK |
| SEQ ID NO: | DNA VL | GAGATTGTTCTTACGCAAAGTCCCGCCACACTTAGTTTGTC |
| 127 | | ACCAGGAGAGCGCGCCACCCTGAGCTGCAGAGCTTCAGA |
| | | GAGTGTGGAATACTACGGCACATCCCTGATGCAGTGGTAT |
| | | CAGCAGAAACCAGGACAGGCTCCTCGGCTGCTGATCTACG |
| | | CAGCCAGCAACGTCGAGTCCGGCATTCCAGCCAGATTTTC |
| | | TGGGTCAGGATCTGGAACTGACTTTACACTGACAATCTCCA |
| | | GCCTGGAACCCGAGGACATTGCTGTGTATTTTTGTCAACAG |
| | | TCCCGGAAGGACCCCAGTACCTTTGGAGGTGGAACCAAGG |
| | | TAGAGATAAAG |
| SEQ ID NO: 58 | Light | EIVLTQSPATLSLSPGERATLSCRASESVEYYGTSLMQWYQQ |
| | Chain | KPGQAPRLLIYAASNVESGIPARFSGSGSGTDFTLTISSLEPED |
| | | IAVYFCQQSRKDPSTFGGGTKVEIKRTVAAPSVFIFPPSDEQL |
| | | KSGTASVVCLLNNFYPREAKVQWKVDNALQSGNSQESVTEQ |
| | | DSKDSTYSLSSTLTLSKADYEKHKVYACEVTHQGLSSPVTKSF |
| | | NRGEC |
| SEQ ID NO: | DNA Light | GAGATTGTTCTTACGCAAAGTCCCGCCACACTTAGTTTGTC |
| 128 | Chain | ACCAGGAGAGCGCGCCACCCTGAGCTGCAGAGCTTCAGA |
| | | GAGTGTGGAATACTACGGCACATCCCTGATGCAGTGGTAT |
| | | CAGCAGAAACCAGGACAGGCTCCTCGGCTGCTGATCTACG |
| | | CAGCCAGCAACGTCGAGTCCGGCATTCCAGCCAGATTTTC |
| | | TGGGTCAGGATCTGGAACTGACTTTACACTGACAATCTCCA |
| | | GCCTGGAACCCGAGGACATTGCTGTGTATTTTTGTCAACAG |
| | | TCCCGGAAGGACCCCAGTACCTTTGGAGGTGGAACCAAGG |
| | | TAGAGATAAAGCGTACGGTGGCAGCTCCGTCTGTTTTCATC |
| | | TTTCCACCTAGCGACGAGCAACTCAAAAGTGGTACAGCATC |
| | | CGTGGTTTGTCTGCTGAACAATTTTTACCCCAGGGAGGCTA |
| | | AGGTCCAGTGGAAAGTCGATAACGCTCTTCAGTCTGGCAA |
| | | CAGTCAGGAGAGCGTCACAGAGCAGGACTCTAAGGATAGC |
| | | ACTTATAGTCTGTCCTCCACGCTGACACTGTCTAAAGCGGA |
| | | TTATGAGAAGCACAAGGTTTACGCCTGTGAGGTAACGCAC |
| | | CAAGGACTCTCCTCCCCAGTTACCAAATCTTTCAACAGAGG |
| | | AGAATGT |
| ABTIM3- | | |
| hum18 | | |
| SEQ ID NO: 3 | HCDR1 | SYNMH |
| (Kabat) | | |
| SEQ ID NO: 30 | HCDR2 | DIYPGQGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 31 | HCDR2 | YPGQGD |
| (Chothia) | | |
| SEQ ID NO: 35 | HCDR3 | VGGAFPMDY |
| (Chothia) | | |
| SEQ ID NO: 72 | VH | QVQLVQSGAEVKKPGSSVKVSCKASGYTFTSYNMHWVRQA |
| | | PGQGLEWMGDIYPGQGDTSYNQKFKGRVTITADKSTSTVYM |
| | | ELSSLRSEDTAVYYCARVGGAFPMDYWGQGTTVTVSS |
| SEQ ID NO: 73 | DNA VH | CAGGTGCAATTGGTTCAGTCAGGAGCAGAAGTTAAGAAGC |
| | | CAGGATCATCCGTCAAGGTGTCCTGCAAAGCATCTGGCTA |
| | | CACCTTCACCAGCTACAATATGCACTGGGTCCGACAAGCC |
| | | CCTGGGCAGGGCTTGGAGTGGATGGGAGACATTTACCCC |
| | | GGCCAGGGTGACACTTCCTATAACCAGAAGTTCAAGGGCC |
| | | GAGTCACTATTACCGCTGACAAGTCCACCTCCACAGTCTAC |
| | | ATGGAACTCTCTTCTCTGAGATCCGAGGACACTGCCGTCTA |
| | | TTACTGCGCTCGCGTGGGCGGTGCTTTCCCAATGGACTAT |
| | | TGGGGACAGGGCACAACCGTGACCGTCAGCTCA |
| SEQ ID NO: 74 | Heavy | QVQLVQSGAEVKKPGSSVKVSCKASGYTFTSYNMHWVRQA |
| | Chain | PGQGLEWMGDIYPGQGDTSYNQKFKGRVTITADKSTSTVYM |
| | | ELSSLRSEDTAVYYCARVGGAFPMDYWGQGTTVTVSSASTK |
| | | GPSVFPLAPCSRSTSESTAALGCLVKDYFPEPVTVSWNSGAL |
| | | TSGVHTFPAVLQSSGLYSLSSVVTVPSSSLGTKTYTCNVDHK |
| | | PSNTKVDKRVESKYGPPCPPCPAPEFLGGPSVFLFPPKPKDT |
| | | LMISRTPEVTCVVVDVSQEDPEVQFNWYVDGVEVHNAKTKP |
| | | REEQFNSTYRVVSVLTVLHQDWLNGKEYKCKVSNKGLPSSIE |
| | | KTISKAKGQPREPQVYTLPPSQEEMTKNQVSLTCLVKGFYPS |
| | | DIAVEWESNGQPENNYKTTPPVLDSDGSFFLYSRLTVDKSRW |
| | | QEGNVFSCSVMHEALHNHYTQKSLSLSLGK |
| SEQ ID NO: 75 | DNA | CAGGTGCAATTGGTTCAGTCAGGAGCAGAAGTTAAGAAGC |
| | Heavy | CAGGATCATCCGTCAAGGTGTCCTGCAAAGCATCTGGCTA |
| | Chain | CACCTTCACCAGCTACAATATGCACTGGGTCCGACAAGCC |
| | | CCTGGGCAGGGCTTGGAGTGGATGGGAGACATTTACCCC |
| | | GGCCAGGGTGACACTTCCTATAACCAGAAGTTCAAGGGCC |
| | | GAGTCACTATTACCGCTGACAAGTCCACCTCCACAGTCTAC |
| | | ATGGAACTCTCTTCTCTGAGATCCGAGGACACTGCCGTCTA |
| | | TTACTGCGCTCGCGTGGGCGGTGCTTTCCCAATGGACTAT |
| | | TGGGGACAGGGCACAACCGTGACCGTCAGCTCAGCCTCTA |
| | | CAAAGGGCCCCTCCGTCTTTCCACTCGCGCCGTGCTCTCG |
| | | CTCCACCTCAGAGTCAACTGCCGCTCTGGGTTGCCTGGTC |
| | | AAGGACTACTTCCCAGAGCCGGTGACAGTGAGCTGGAACA |
| | | GTGGGGCCCTGACATCCGGCGTTCATACCTTCCCCGCAGT |
| | | CCTCCAGTCCTCAGGCCTGTATTCCCTGAGCAGCGTTGTC |
| | | ACAGTGCCCTCCAGCTCTCTTGGCACGAAAACCTACACAT |
| | | GCAACGTTGATCATAAGCCGTCTAATACCAAGGTGGATAAA |
| | | AGAGTGGAGAGCAAGTACGGCCCACCCTGCCCGCCTTGC |
| | | CCAGCTCCGGAGTTCCTGGGCGGACCATCCGTTTTCTTGT |
| | | TTCCACCCAAACCTAAAGACACTCTGATGATTTCCCGAACC |
| | | CCTGAAGTGACTTGCGTTGTGGTGGACGTCTCCCAGGAGG |
| | | ACCCAGAAGTGCAATTCAACTGGTACGTGGACGGGGTGGA |
| | | GGTGCACAATGCAAAAACCAAACCAAGGGAGGAACAGTTT |
| | | AATTCAACATATAGGGTTGTGTCTGTGCTGACGGTTCTGCA |
| | | TCAGGACTGGCTGAACGGAAAGGAATACAAGTGCAAGGTG |
| | | TCCAACAAAGGACTGCCAAGCTCTATCGAGAAAACAATCTC |
| | | TAAGGCCAAGGGACAACCTAGAGAGCCCCAAGTTTACACC |
| | | CTGCCACCATCACAGGAAGAGATGACCAAAAATCAGGTGA |
| | | GCTTGACATGCCTGGTGAAGGGCTTCTACCCTAGCGATATT |
| | | GCGGTTGAGTGGGAGTCAAATGGCCAGCCTGAGAACAACT |
| | | ATAAGACTACTCCTCCCGTGCTGGACTCCGACGGGAGCTT |
| | | TTTCCTGTATTCCAGGCTTACAGTCGATAAGAGCAGATGGC |
| | | AAGAGGGGAATGTGTTTTCCTGCTCCGTGATGCACGAGGC |
| | | TCTCCATAACCATTATACTCAGAAAAGTCTCTCTCTGTCACT |
| | | GGGCAAA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 56 | VL | EIVLTQSPATLSLSPGERATLSCRASESVEYYGTSLMQWYQQ |
| | | KPGQAPRLLIYAASNVESGIPARFSGSGSGTDFTLTISSLEPED |
| | | IAVYFCQQSRKDPSTFGGGTKVEIK |
| SEQ ID NO: 1 | DNA VL | GAGATTGTTCTTACGCAAAGTCCCGCCACACTTAGTTTGTC |
| 127 | | ACCAGGAGAGCGCGCCACCCTGAGCTGCAGAGCTTCAGA |
| | | GAGTGTGGAATACTACGGCACATCCCTGATGCAGTGGTAT |
| | | CAGCAGAAACCAGGACAGGCTCCTCGGCTGCTGATCTACG |
| | | CAGCCAGCAACGTCGAGTCCGGCATTCCAGCCAGATTTTC |
| | | TGGGTCAGGATCTGGAACTGACTTTACACTGACAATCTCCA |
| | | GCCTGGAACCCGAGGACATTGCTGTGTATTTTTGTCAACAG |
| | | TCCCGGAAGGACCCCAGTACCTTTGGAGGTGGAACCAAGG |
| | | TAGAGATAAAG |
| SEQ ID NO: 58 | Light | EIVLTQSPATLSLSPGERATLSCRASESVEYYGTSLMQWYQQ |
| | Chain | KPGQAPRLLIYAASNVESGIPARFSGSGSGTDFTLTISSLEPED |
| | | IAVYFCQQSRKDPSTFGGGTKVEIKRTVAAPSVFIFPPSDEQL |
| | | KSGTASVVCLLNNFYPREAKVQWKVDNALQSGNSQESVTEQ |
| | | DSKDSTYSLSSTLTLSKADYEKHKVYACEVTHQGLSSPVTKSF |
| | | NRGEC |
| SEQ ID NO: | DNA Light | GAGATTGTTCTTACGCAAAGTCCCGCCACACTTAGTTTGTC |
| 128 | Chain | ACCAGGAGAGCGCGCCACCCTGAGCTGCAGAGCTTCAGA |
| | | GAGTGTGGAATACTACGGCACATCCCTGATGCAGTGGTAT |
| | | CAGCAGAAACCAGGACAGGCTCCTCGGCTGCTGATCTACG |
| | | CAGCCAGCAACGTCGAGTCCGGCATTCCAGCCAGATTTTC |
| | | TGGGTCAGGATCTGGAACTGACTTTACACTGACAATCTCCA |
| | | GCCTGGAACCCGAGGACATTGCTGTGTATTTTTGTCAACAG |
| | | TCCCGGAAGGACCCCAGTACCTTTGGAGGTGGAACCAAGG |
| | | TAGAGATAAAGCGTACGGTGGCAGCTCCGTCTGTTTTCATC |
| | | TTTCCACCTAGCGACGAGCAACTCAAAAGTGGTACAGCATC |
| | | CGTGGTTTGTCTGCTGAACAATTTTTACCCCAGGGAGGCTA |
| | | AGGTCCAGTGGAAAGTCGATAACGCTCTTCAGTCTGGCAA |
| | | CAGTCAGGAGAGCGTCACAGAGCAGGACTCTAAGGATAGC |
| | | ACTTATAGTCTGTCCTCCACGCTGACACTGTCTAAAGCGGA |
| | | TTATGAGAAGCACAAGGTTTACGCCTGTGAGGTAACGCAC |
| | | CAAGGACTCTCCTCCCCAGTTACCAAATCTTTCAACAGAGG |
| | | AGAATGT |
| ABTIM3- | | |
| hum19 | | |
| SEQ ID NO: 3 | HCDR1 | SYNMH |
| (Kabat) | | |
| SEQ ID NO: 24 | HCDR2 | DIYPGSGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 25 | HCDR2 | YPGSGD |
| (Chothia) | | |
| SEQ ID NO: 5 | HCDR3 | VGGAFPMDY |
| (Chothia) | | |
| SEQ ID NO: 76 | VH | EVQLVQSGAEVKKPGESLKISCKGSGYTFTSYNMHWVRQMP |
| | | GKGLEWMGDIYPGSGDTSYNQKFKGQVTISADKSISTVYLQW |
| | | SSLKASDTAMYYCARVGGAFPMDYWGQGTTVTVSS |
| SEQ ID NO: 77 | DNA VH | GAAGTTCAATTGGTACAGTCTGGCGCAGAAGTAAAGAAAC |
| | | CAGGAGAGAGTTTGAAAATTTCCTGCAAGGGCAGTGGGTA |
| | | CACATTCACGTCCTACAATATGCACTGGGTGAGACAGATGC |
| | | CAGGCAAGGGCCTGGAGTGGATGGGAGACATATACCCAG |
| | | GCAGTGGAGACACAAGCTATAATCAGAAATTCAAAGGACA |
| | | GGTGACGATCTCCGCAGACAAATCCATATCTACGGTCTACC |
| | | TCCAGTGGTCCTCACTTAAAGCCTCCGACACCGCCATGTA |
| | | CTATTGCGCTCGGGTAGGTGGCGCGTTTCCAATGGACTAT |
| | | TGGGGCCAAGGGACCACAGTAACCGTCAGCTCA |
| SEQ ID NO: 78 | Heavy | EVQLVQSGAEVKKPGESLKISCKGSGYTFTSYNMHWVRQMP |
| | Chain | GKGLEWMGDIYPGSGDTSYNQKFKGQVTISADKSISTVYLQW |
| | | SSLKASDTAMYYCARVGGAFPMDYWGQGTTVTVSSASTKGP |
| | | SVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQE |
| | | GNVFSCSVMHEALHNHYTQKSLSLSLGK |
| SEQ ID NO: 79 | DNA | GAAGTTCAATTGGTACAGTCTGGCGCAGAAGTAAAGAAAC |
| | Heavy | CAGGAGAGAGTTTGAAAATTTCCTGCAAGGGCAGTGGGTA |
| | Chain | CACATTCACGTCCTACAATATGCACTGGGTGAGACAGATGC |
| | | CAGGCAAGGGCCTGGAGTGGATGGGAGACATATACCCAG |
| | | GCAGTGGAGACACAAGCTATAATCAGAAATTCAAAGGACA |
| | | GGTGACGATCTCCGCAGACAAATCCATATCTACGGTCTACC |
| | | TCCAGTGGTCCTCACTTAAAGCCTCCGACACCGCCATGTA |
| | | CTATTGCGCTCGGGTAGGTGGCGCGTTTCCAATGGACTAT |
| | | TGGGGCCAAGGGACCACAGTAACCGTCAGCTCAGCCTCTA |
| | | CAAAGGGCCCCTCCGTCTTTCCACTCGCGCCGTGCTCTCG |
| | | CTCCACCTCAGAGTCAACTGCCGCTCTGGGTTGCCTGGTC |
| | | AAGGACTACTTCCCAGAGCCGGTGACAGTGAGCTGGAACA |
| | | GTGGGGCCCTGACATCCGGCGTTCATACCTTCCCCGCAGT |
| | | CCTCCAGTCCTCAGGCCTGTATTCCCTGAGCAGCGTTGTC |
| | | ACAGTGCCCTCCAGCTCTCTTGGCACGAAAACCTACACAT |
| | | GCAACGTTGATCATAAGCCGTCTAATACCAAGGTGGATAAA |
| | | AGAGTGGAGAGCAAGTACGGCCCACCCTGCCCGCCTTGC |
| | | CCAGCTCCGGAGTTCCTGGGCGGACCATCCGTTTTCTTGT |
| | | TTCCACCCAAACCTAAAGACACTCTGATGATTTCCCGAACC |
| | | CCTGAAGTGACTTGCGTTGTGGTGGACGTCTCCCAGGAGG |
| | | ACCCAGAAGTGCAATTCAACTGGTACGTGGACGGGGTGGA |
| | | GGTGCACAATGCAAAAACCAAACCAAGGGAGGAACAGTTT |
| | | AATTCAACATATAGGGTTGTGTCTGTGCTGACGGTTCTGCA |
| | | TCAGGACTGGCTGAACGGAAAGGAATACAAGTGCAAGGTG |
| | | TCCAACAAAGGACTGCCAAGCTCTATCGAGAAAACAATCTC |
| | | TAAGGCCAAGGGACAACCTAGAGAGCCCCAAGTTTACACC |
| | | CTGCCACCATCACAGGAAGAGATGACCAAAAATCAGGTGA |
| | | GCTTGACATGCCTGGTGAAGGGCTTCTACCCTAGCGATATT |
| | | GCGGTTGAGTGGGAGTCAAATGGCCAGCCTGAGAACAACT |
| | | ATAAGACTACTCCTCCCGTGCTGGACTCCGACGGGAGCTT |
| | | TTTCCTGTATTCCAGGCTTACAGTCGATAAGAGCAGATGGC |
| | | AAGAGGGGAATGTGTTTTCCTGCTCCGTGATGCACGAGGC |
| | | TCTCCATAACCATTATACTCAGAAAAGTCTCTCTCTGTCACT |
| | | GGGCAAA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 64 | VL | AIQLTQSPSSLSASVGDRVTITCRASESVEYYGTSLMQWYQQ |
| | | KPGKAPKLLIYAASNVESGVPSRFSGSGSGTDFTLTISSLQPE |
| | | DFATYFCQQSRKDPSTFGGGTKVEIK |
| SEQ ID NO: | DNA VL | GCAATACAGTTGACACAGAGTCCTTCAAGTTTGTCCGCTTC |
| 125 | | CGTTGGCGACCGAGTGACAATCACCTGTAGAGCATCCGAG |
| | | TCAGTGGAGTATTATGGCACTAGCCTGATGCAGTGGTATCA |
| | | GCAAAAGCCAGGGAAAGCCCCAAAGCTGCTGATATATGCC |
| | | GCGAGTAACGTCGAGTCAGGGGTGCCATCAAGATTCTCCG |
| | | GTTCCGGGTCCGGAACCGACTTCACACTGACCATCTCTTC |
| | | CCTTCAGCCAGAGGACTTCGCTACGTACTTTTGCCAGCAGT |
| | | CACGGAAAGATCCCTCTACTTTCGGAGGTGGGACAAAAGT |
| | | CGAAATTAAA |
| SEQ ID NO: 66 | Light | AIQLTQSPSSLSASVGDRVTITCRASESVEYYGTSLMQWYQQ |
| | Chain | KPGKAPKLLIYAASNVESGVPSRFSGSGSGTDFTLTISSLQPE |
| | | DFATYFCQQSRKDPSTFGGGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: | DNA Light | GCAATACAGTTGACACAGAGTCCTTCAAGTTTGTCCGCTTC |
| 126 | Chain | CGTTGGCGACCGAGTGACAATCACCTGTAGAGCATCCGAG |
| | | TCAGTGGAGTATTATGGCACTAGCCTGATGCAGTGGTATCA |
| | | GCAAAAGCCAGGGAAAGCCCCAAAGCTGCTGATATATGCC |
| | | GCGAGTAACGTCGAGTCAGGGGTGCCATCAAGATTCTCCG |
| | | GTTCCGGGTCCGGAACCGACTTCACACTGACCATCTCTTC |
| | | CCTTCAGCCAGAGGACTTCGCTACGTACTTTTGCCAGCAGT |
| | | CACGGAAAGATCCCTCTACTTTCGGAGGTGGGACAAAAGT |
| | | CGAAATTAAACGTACGGTGGCAGCTCCGTCTGTTTTCATCT |
| | | TTCCACCTAGCGACGAGCAACTCAAAAGTGGTACAGCATC |
| | | CGTGGTTTGTCTGCTGAACAATTTTTACCCCAGGGAGGCTA |
| | | AGGTCCAGTGGAAAGTCGATAACGCTCTTCAGTCTGGCAA |
| | | CAGTCAGGAGAGCGTCACAGAGCAGGACTCTAAGGATAGC |
| | | ACTTATAGTCTGTCCTCCACGCTGACACTGTCTAAAGCGGA |
| | | TTATGAGAAGCACAAGGTTTACGCCTGTGAGGTAACGCAC |
| | | CAAGGACTCTCCTCCCCAGTTACCAAATCTTTCAACAGAGG |
| | | AGAATGT |
| ABTIM3- | | |
| hum20 | | |
| SEQ ID NO: 3 | HCDR1 | SYNMH |
| (Kabat) | | |
| SEQ ID NO: 30 | HCDR2 | DIYPGQGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 31 | HCDR2 | YPGQGD |
| (Chothia) | | |
| SEQ ID NO: 5 | HCDR3 | VGGAFPMDY |
| (Chothia) | | |
| SEQ ID NO: 80 | VH | EVQLVQSGAEVKKPGESLKISCKGSGYTFTSYNMHWVRQMP |
| | | GKGLEWMGDIYPGQGDTSYNQKFKGQVTISADKSISTVYLQW |
| | | SSLKASDTAMYYCARVGGAFPMDYWGQGTTVTVSS |
| SEQ ID NO: 81 | DNA VH | GAAGTTCAATTGGTACAGTCTGGCGCAGAAGTAAAGAAAC |
| | | CAGGAGAGAGTTTGAAAATTTCCTGCAAGGGCAGTGGGTA |
| | | CACATTCACGTCCTACAATATGCACTGGGTGAGACAGATGC |
| | | CAGGCAAGGGCCTGGAGTGGATGGGAGACATATACCCAG |
| | | GCCAGGGAGACACAAGCTATAATCAGAAATTCAAAGGACA |
| | | GGTGACGATCTCCGCAGACAAATCCATATCTACGGTCTACC |
| | | TCCAGTGGTCCTCACTTAAAGCCTCCGACACCGCCATGTA |
| | | CTATTGCGCTCGGGTAGGTGGCGCGTTTCCAATGGACTAT |
| | | TGGGGCCAAGGGACCACAGTAACCGTCAGCTCA |
| SEQ ID NO: 82 | Heavy | EVQLVQSGAEVKKPGESLKISCKGSGYTFTSYNMHWVRQMP |
| | Chain | GKGLEWMGDIYPGQGDTSYNQKFKGQVTISADKSISTVYLQW |
| | | SSLKASDTAMYYCARVGGAFPMDYWGQGTTVTVSSASTKGP |
| | | SVFPLAPCSRSTSESTAALGCLVKDYFPEPVTVSWNSGALTS |
| | | GVHTFPAVLQSSGLYSLSSVVTVPSSSLGTKTYTCNVDHKPS |
| | | NTKVDKRVESKYGPPCPPCPAPEFLGGPSVFLFPPKPKDTLM |
| | | ISRTPEVTCVVVDVSQEDPEVQFNWYVDGVEVHNAKTKPRE |
| | | EQFNSTYRVVSVLTVLHQDWLNGKEYKCKVSNKGLPSSIEKTI |
| | | SKAKGQPREPQVYTLPPSQEEMTKNQVSLTCLVKGFYPSDIA |
| | | VEWESNGQPENNYKTTPPVLDSDGSFFLYSRLTVDKSRWQE |
| | | GNVFSCSVMHEALHNHYTQKSLSLSLGK |
| SEQ ID NO: 83 | DNA | GAAGTTCAATTGGTACAGTCTGGCGCAGAAGTAAAGAAAC |
| | Heavy | CAGGAGAGAGTTTGAAAATTTCCTGCAAGGGCAGTGGGTA |
| | Chain | CACATTCACGTCCTACAATATGCACTGGGTGAGACAGATGC |
| | | CAGGCAAGGGCCTGGAGTGGATGGGAGACATATACCCAG |
| | | GCCAGGGAGACACAAGCTATAATCAGAAATTCAAAGGACA |
| | | GGTGACGATCTCCGCAGACAAATCCATATCTACGGTCTACC |
| | | TCCAGTGGTCCTCACTTAAAGCCTCCGACACCGCCATGTA |
| | | CTATTGCGCTCGGGTAGGTGGCGCGTTTCCAATGGACTAT |
| | | TGGGGCCAAGGGACCACAGTAACCGTCAGCTCAGCCTCTA |
| | | CAAAGGGCCCCTCCGTCTTTCCACTCGCGCCGTGCTCTCG |
| | | CTCCACCTCAGAGTCAACTGCCGCTCTGGGTTGCCTGGTC |
| | | AAGGACTACTTCCCAGAGCCGGTGACAGTGAGCTGGAACA |
| | | GTGGGGCCCTGACATCCGGCGTTCATACCTTCCCCGCAGT |
| | | CCTCCAGTCCTCAGGCCTGTATTCCCTGAGCAGCGTTGTC |
| | | ACAGTGCCCTCCAGCTCTCTTGGCACGAAAACCTACACAT |
| | | GCAACGTTGATCATAAGCCGTCTAATACCAAGGTGGATAAA |
| | | AGAGTGGAGAGCAAGTACGGCCCACCCTGCCCGCCTTGC |
| | | CCAGCTCCGGAGTTCCTGGGCGGACCATCCGTTTTCTTGT |
| | | TTCCACCCAAACCTAAAGACACTCTGATGATTTCCCGAACC |
| | | CCTGAAGTGACTTGCGTTGTGGTGGACGTCTCCCAGGAGG |
| | | ACCCAGAAGTGCAATTCAACTGGTACGTGGACGGGGTGGA |
| | | GGTGCACAATGCAAAAACCAAACCAAGGGAGGAACAGTTT |
| | | AATTCAACATATAGGGTTGTGTCTGTGCTGACGGTTCTGCA |
| | | TCAGGACTGGCTGAACGGAAAGGAATACAAGTGCAAGGTG |
| | | TCCAACAAAGGACTGCCAAGCTCTATCGAGAAAACAATCTC |
| | | TAAGGCCAAGGGACAACCTAGAGAGCCCCAAGTTTACACC |
| | | CTGCCACCATCACAGGAAGAGATGACCAAAAATCAGGTGA |
| | | GCTTGACATGCCTGGTGAAGGGCTTCTACCCTAGCGATATT |
| | | GCGGTTGAGTGGGAGTCAAATGGCCAGCCTGAGAACAACT |
| | | ATAAGACTACTCCTCCCGTGCTGGACTCCGACGGGAGCTT |
| | | TTTCCTGTATTCCAGGCTTACAGTCGATAAGAGCAGATGGC |
| | | AAGAGGGGAATGTGTTTTCCTGCTCCGTGATGCACGAGGC |
| | | TCTCCATAACCATTATACTCAGAAAAGTCTCTCTCTGTCACT |
| | | GGGCAAA |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 64 | VL | AIQLTQSPSSLSASVGDRVTITCRASESVEYYGTSLMQWYQQ |
| | | KPGKAPKLLIYAASNVESGVPSRFSGSGSGTDFTLTISSLQPE |
| | | DFATYFCQQSRKDPSTFGGGTKVEIK |
| SEQ ID NO: | DNA VL | GCAATACAGTTGACACAGAGTCCTTCAAGTTTGTCCGCTTC |
| 125 | | CGTTGGCGACCGAGTGACAATCACCTGTAGAGCATCCGAG |
| | | TCAGTGGAGTATTATGGCACTAGCCTGATGCAGTGGTATCA |
| | | GCAAAAGCCAGGGAAAGCCCCAAAGCTGCTGATATATGCC |
| | | GCGAGTAACGTCGAGTCAGGGGTGCCATCAAGATTCTCCG |
| | | GTTCCGGGTCCGGAACCGACTTCACACTGACCATCTCTTC |
| | | CCTTCAGCCAGAGGACTTCGCTACGTACTTTTGCCAGCAGT |
| | | CACGGAAAGATCCCTCTACTTTCGGAGGTGGGACAAAAGT |
| | | CGAAATTAAA |
| SEQ ID NO: 66 | Light | AIQLTQSPSSLSASVGDRVTITCRASESVEYYGTSLMQWYQQ |
| | Chain | KPGKAPKLLIYAASNVESGVPSRFSGSGSGTDFTLTISSLQPE |
| | | DFATYFCQQSRKDPSTFGGGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: | DNA Light | GCAATACAGTTGACACAGAGTCCTTCAAGTTTGTCCGCTTC |
| 126 | Chain | CGTTGGCGACCGAGTGACAATCACCTGTAGAGCATCCGAG |
| | | TCAGTGGAGTATTATGGCACTAGCCTGATGCAGTGGTATCA |
| | | GCAAAAGCCAGGGAAAGCCCCAAAGCTGCTGATATATGCC |
| | | GCGAGTAACGTCGAGTCAGGGGTGCCATCAAGATTCTCCG |
| | | GTTCCGGGTCCGGAACCGACTTCACACTGACCATCTCTTC |
| | | CCTTCAGCCAGAGGACTTCGCTACGTACTTTTGCCAGCAGT |
| | | CACGGAAAGATCCCTCTACTTTCGGAGGTGGGACAAAAGT |
| | | CGAAATTAAACGTACGGTGGCAGCTCCGTCTGTTTTCATCT |
| | | TTCCACCTAGCGACGAGCAACTCAAAAGTGGTACAGCATC |
| | | CGTGGTTTGTCTGCTGAACAATTTTTACCCCAGGGAGGCTA |
| | | AGGTCCAGTGGAAAGTCGATAACGCTCTTCAGTCTGGCAA |
| | | CAGTCAGGAGAGCGTCACAGAGCAGGACTCTAAGGATAGC |
| | | ACTTATAGTCTGTCCTCCACGCTGACACTGTCTAAAGCGGA |
| | | TTATGAGAAGCACAAGGTTTACGCCTGTGAGGTAACGCAC |
| | | CAAGGACTCTCCTCCCCAGTTACCAAATCTTTCAACAGAGG |
| | | AGAATGT |
| ABTIM3- | | |
| hum21 | | |
| SEQ ID NO: 3 | HCDR1 | SYNMH |
| (Kabat) | | |
| SEQ ID NO: 30 | HCDR2 | DIYPGQGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 31 | HCDR2 | YPGQGD |
| (Chothia) | | |
| SEQ ID NO: 5 | HCDR3 | VGGAFPMDY |
| (Chothia) | | |
| SEQ ID NO: 84 | VH | QVQLVQSGAEVKKPGASVKVSCKASGYTFTSYNMHWVRQA |
| | | PGQGLEWIGDIYPGQGDTSYNQKFKGRATMTADKSTSTVYM |
| | | ELSSLRSEDTAVYYCARVGGAFPMDYWGQGTLVTVSS |
| SEQ ID NO: 85 | DNA VH | CAGGTGCAATTGGTGCAGAGCGGAGCAGAGGTCAAAAAGC |
| | | CCGGAGCAAGCGTGAAGGTCTCATGCAAAGCAAGCGGATA |
| | | CACATTTACATCATACAACATGCACTGGGTCAGGCAGGCTC |
| | | CAGGACAGGGACTGGAGTGGATCGGGGACATCTACCCTG |
| | | GACAGGGCGATACTAGCTATAATCAGAAGTTCAAAGGCCG |
| | | GGCCACCATGACAGCTGACAAGTCTACTAGTACCGTGTATA |
| | | TGGAACTGAGCTCCCTGCGGTCTGAAGATACCGCAGTGTA |
| | | CTATTGCGCCAGAGTCGGGGGGGCATTTCCTATGGATTAT |
| | | TGGGGGCAGGGGACTCTGGTCACTGTCAGCTCA |
| SEQ ID NO: 86 | Heavy | QVQLVQSGAEVKKPGASKVKSCKASGYTFTSYNMHWVRQA |
| | Chain | PGQGLEWIGDIYPGQGDTSYNQKFKGRATMTADKSTSTVYM |
| | | ELSSLRSEDTAVYYCARVGGAFPMDYWGQGTLVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHKP |
| | | SNTKVDKRVEPKSCDKTHTCPPCPAPELLGGPSVFLFPPKPK |
| | | DTLMISRTPEVTCVVVAVSHEDPEVKFNWYVDGVEVHNAKTK |
| | | PREEQYNSTYRVVSVLTVLHQDWLNGKEYKCKVSNKALAAPI |
| | | EKTISKAKGQPREPQVYTLPPSREEMTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGK |
| SEQ ID NO: 87 | DNA | CAGGTGCAATTGGTGCAGAGCGGAGCAGAGGTCAAAAAGC |
| | Heavy | CCGGAGCAAGCGTGAAGGTCTCATGCAAAGCAAGCGGATA |
| | Chain | CACATTTACATCATACAACATGCACTGGGTCAGGCAGGCTC |
| | | CAGGACAGGGACTGGAGTGGATCGGGGACATCTACCCTG |
| | | GACAGGGCGATACTAGCTATAATCAGAAGTTCAAAGGCCG |
| | | GGCCACCATGACAGCTGACAAGTCTACTAGTACCGTGTATA |
| | | TGGAACTGAGCTCCCTGCGGTCTGAAGATACCGCAGTGTA |
| | | CTATTGCGCCAGAGTCGGGGGGGCATTTCCTATGGATTAT |
| | | TGGGGGCAGGGGACTCTGGTCACTGTCAGCTCAGCTAGCA |
| | | CCAAGGGCCCCAGCGTGTTCCCCCTGGCCCCCAGCAGCA |
| | | AGAGCACCAGCGGCGGCACAGCCGCCCTGGGCTGCCTGG |
| | | TGAAGGACTACTTCCCCGAGCCCGTGACCGTGTCCTGGAA |
| | | CAGCGGAGCCCTGACCTCCGGCGTGCACACCTTCCCCGC |
| | | CGTGCTGCAGAGCAGCGGCCTGTACAGCCTGTCCAGCGT |
| | | GGTGACAGTGCCCAGCAGCAGCCTGGGCACCCAGACCTA |
| | | CATCTGCAACGTGAACCACAAGCCCAGCAACACCAAGGTG |
| | | GACAAGAGAGTGGAGCCCAAGAGCTGCGACAAGACCCAC |
| | | ACCTGCCCCCCCTGCCCAGCCCCAGAGCTGCTGGGCGGA |
| | | CCCTCCGTGTTCCTGTTCCCCCCCAAGCCCAAGGACACCC |
| | | TGATGATCAGCAGGACCCCCGAGGTGACCTGCGTGGTGGT |
| | | GGCCGTGAGCCACGAGGACCCAGAGGTGAAGTTCAACTG |
| | | GTACGTGGACGGCGTGGAGGTGCACAACGCCAAGACCAA |
| | | GCCCAGAGAGGAGCAGTACAACAGCACCTACAGGGTGGT |
| | | GTCCGTGCTGACCGTGCTGCACCAGGACTGGCTGAACGG |
| | | CAAGGAATACAAGTGCAAGGTCTCCAACAAGGCCCTGGCA |
| | | GCCCCCATCGAAAAGACCATCAGCAAGGCCAAGGGCCAG |
| | | CCACGGGAGCCCCAGGTGTACACCCTGCCCCCCTCCCGG |
| | | GAGGAGATGACCAAGAACCAGGTGTCCCTGACCTGTCTGG |
| | | TGAAGGGCTTCTACCCCAGCGACATCGCCGTGGAGTGGGA |
| | | GAGCAACGGCCAGCCCGAGAACAACTACAAGACCACCCCC |
| | | CCAGTGCTGGACAGCGACGGCAGCTTCTTCCTGTACAGCA |
| | | AGCTGACCGTGGACAAGTCCAGGTGGCAGCAGGGCAACG |
| | | TGTTCAGCTGCAGCGTGATGCACGAGGCCCTGCACAACCA |
| | | CTACACCCAGAAGAGCCTGAGCCTGTCCCCCGGCAAG |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 88 | VL | DIVLTQSPDSLAVSLGERATINCRASESVEYYGTSLMQWYWW |
| | | KPGQPPKLLIYAASNVESGVPDRFSGSGSGTDFTLTISSLQAE |
| | | DVAVYYCQQSRKDPSTFGGGTKVEIK |
| SEQ ID NO: 89 | DNA VL | GACATCGTCCTGACACAGTCTCCTGACAGCCTGGCAGTGA |
| | | GCCTGGGCGAAAGGGCAACCATTAATTGTAGAGCTTCCGA |
| | | GTCCGTCGAGTACTATGGCACTAGTCTGATGCAGTGGTAC |
| | | CAGCAGAAGCCAGGGCAGCCCCCTAAACTGCTGATCTATG |
| | | CAGCTAGCAACGTGGAGTCCGGAGTCCCAGACCGGTTCTC |
| | | TGGAAGTGGGTCAGGAACCGATTTTACCCTGACAATTAGCT |
| | | CCCTGCAGGCAGAAGACGTGGCCGTCTACTATTGTCAGCA |
| | | GAGCCGCAAGGACCCAAGCACATTCGGAGGGGGGACCAA |
| | | AGTGGAAATCAAG |
| SEQ ID NO: 90 | Light | DIVLTQSPDSLAVSLGERATINCRASESVEYYGTSLMQWYQQ |
| | Chain | KPGQPPKLLIYAASNVESGVPDRFSGSGSGTDFTLTISSLQAE |
| | | DVAVYYCQQSRKDPSTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| SEQ ID NO: 91 | DNA Light | GACATCGTCCTGACACAGTCTCCTGACAGCCTGGCAGTGA |
| | Chain | GCCTGGGCGAAAGGGCAACCATTAATTGTAGAGCTTCCGA |
| | | GTCCGTCGAGTACTATGGCACTAGTCTGATGCAGTGGTAC |
| | | CAGCAGAAGCCAGGGCAGCCCCCTAAACTGCTGATCTATG |
| | | CAGCTAGCAACGTGGAGTCCGGAGTCCCAGACCGGTTCTC |
| | | TGGAAGTGGGTCAGGAACCGATTTTACCCTGACAATTAGCT |
| | | CCCTGCAGGCAGAAGACGTGGCCGTCTACTATTGTCAGCA |
| | | GAGCCGCAAGGACCCAAGCACATTCGGAGGGGGGACCAA |
| | | AGTGGAAATCAAGCGTACGGTGGCCGCTCCCAGCGTGTTC |
| | | ATCTTCCCCCCCAGCGACGAGCAGCTGAAGAGCGGCACC |
| | | GCCAGCGTGGTGTGCCTGCTGAACAACTTCTACCCCCGGG |
| | | AGGCCAAGGTGCAGTGGAAGGTGGACAACGCCCTGCAGA |
| | | GCGGCAACAGCCAGGAGAGCGTCACCGAGCAGGACAGCA |
| | | AGGACTCCACCTACAGCCTGAGCAGCACCCTGACCCTGAG |
| | | CAAGGCCGACTACGAGAAGCATAAGGTGTACGCCTGCGAG |
| | | GTGACCCACCAGGGCCTGTCCAGCCCCGTGACCAAGAGC |
| | | TTCAACAGGGGCGAGTGC |
| ABTIM3- | | |
| hum22 | | |
| SEQ ID NO: 3 | HCDR1 | SYNMH |
| (Kabat) | | |
| SEQ ID NO: 30 | HCDR2 | DIYPGQGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 31 | HCDR2 | YPGQGD |
| (Chothia) | | |
| SEQ ID NO: 5 | HCDR3 | VGGAFPMDY |
| (Chothia) | | |
| SEQ ID NO: 92 | VH | QVQLVQSGAEVKKPGASVKVSCKASGYTFTSYNMHWVRQA |
| | | PGQGLEWMGDIYPGQGDTSYNQKFKGRVTMTRDTSTSTVY |
| | | MELSSLRSEDTAVYYCARVGGAFPMDYWGQGTTVTVSS |
| SEQ ID NO: 93 | DNA VH | CAGGTGCAGCTGGTGCAGTCTGGCGCCGAAGTGAAGAAA |
| | | CCAGGCGCCAGCGTGAAGGTGTCCTGCAAGGCCAGCGGC |
| | | TACACCTTTACCAGCTACAACATGCACTGGGTGCGCCAGG |
| | | CCCCTGGACAGGGACTGGAATGGATGGGCGACATCTACCC |
| | | CGGCCAGGGCGACACCTCCTACAACCAGAAATTCAAGGGC |
| | | AGAGTGACCATGACCCGGGACACCAGCACCTCCACCGTGT |
| | | ACATGGAACTGAGCAGCCTGCGGAGCGAGGACACCGCCG |
| | | TGTACTACTGTGCTAGAGTGGGCGGAGCCTTCCCCATGGA |
| | | CTATTGGGGCCAGGGCACCACCGTGACCGTGAGCTCA |
| SEQ ID NO: 94 | Heavy | QVQLVQSGAEVKKPGASVKVSCKASGYTFTSYNMHWVRQA |
| | Chain | PGQGLEWMGDIYPGQGDTSYNQKFKGRVTMTRDTSTSTVY |
| | | MELSSLRSEDTAVYYCARVGGAFPMDYWGQGTTVTVSSAST |
| | | KGPSVFPLAPSSKSTSGGTAALGCLVKDYFPEPVTVSWNSGA |
| | | LTSGVHTFPAVLQSSGLYSLSSVVTVPSSSLGTQTYICNVNHK |
| | | PSNTKVDKRVEPKSCDKTHTCPPCPAPELLGGPSVFLFPPKP |
| | | KDTLMISRTPEVTCVVVAVSHEDPEVKFNWYVDGVEVHNAKT |
| | | KPREEQYNSTYRVVSVLTVLHQDWLNGKEYKCKVSNKALAA |
| | | PIEKTISKAKGQPREPQVYTLPPSREEMTKNQVSLTCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| SEQ ID NO: 95 | DNA | CAGGTGCAGCTGGTGCAGTCTGGCGCCGAAGTGAAGAAA |
| | Heavy | CCAGGCGCCAGCGTGAAGGTGTCCTGCAAGGCCAGCGGC |
| | Chain | TACACCTTTACCAGCTACAACATGCACTGGGTGCGCCAGG |
| | | CCCCTGGACAGGGACTGGAATGGATGGGCGACATCTACCC |
| | | CGGCCAGGGCGACACCTCCTACAACCAGAAATTCAAGGGC |
| | | AGAGTGACCATGACCCGGGACACCAGCACCTCCACCGTGT |
| | | ACATGGAACTGAGCAGCCTGCGGAGCGAGGACACCGCCG |
| | | TGTACTACTGTGCTAGAGTGGGCGGAGCCTTCCCCATGGA |
| | | CTATTGGGGCCAGGGCACCACCGTGACCGTGAGCTCAGCT |
| | | AGCACCAAGGGCCCCAGCGTGTTCCCCCTGGCCCCCAGC |
| | | AGCAAGAGCACCAGCGGCGGCACAGCCGCCCTGGGCTGC |
| | | CTGGTGAAGGACTACTTCCCCGAGCCCGTGACCGTGTCCT |
| | | GGAACAGCGGAGCCCTGACCTCCGGCGTGCACACCTTCC |
| | | CCGCCGTGCTGCAGAGCAGCGGCCTGTACAGCCTGTCCA |
| | | GCGTGGTGACAGTGCCCAGCAGCAGCCTGGGCACCCAGA |
| | | CCTACATCTGCAACGTGAACCACAAGCCCAGCAACACCAA |
| | | GGTGGACAAGAGAGTGGAGCCCAAGAGCTGCGACAAGAC |
| | | CCACACCTGCCCCCCCTGCCCAGCCCCAGAGCTGCTGGG |
| | | CGGACCCTCCGTGTTCCTGTTCCCCCCCAAGCCCAAGGAC |
| | | ACCCTGATGATCAGCAGGACCCCCGAGGTGACCTGCGTG |
| | | GTGGTGGCCGTGAGCCACGAGGACCCAGAGGTGAAGTTC |
| | | AACTGGTACGTGGACGGCGTGGAGGTGCACAACGCCAAG |
| | | ACCAAGCCCAGAGAGGAGCAGTACAACAGCACCTACAGGG |
| | | TGGTGTCCGTGCTGACCGTGCTGCACCAGGACTGGCTGAA |
| | | CGGCAAGGAATACAAGTGCAAGGTCTCCAACAAGGCCCTG |
| | | GCAGCCCCCATCGAAAAGACCATCAGCAAGGCCAAGGGC |
| | | CAGCCACGGGAGCCCCAGGTGTACACCCTGCCCCCCTCC |
| | | CGGGAGGAGATGACCAAGAACCAGGTGTCCCTGACCTGTC |
| | | TGGTGAAGGGCTTCTACCCCAGCGACATCGCCGTGGAGTG |
| | | GGAGAGCAACGGCCAGCCCGAGAACAACTACAAGACCAC |
| | | CCCCCCAGTGCTGGACAGCGACGGCAGCTTCTTCCTGTAC |
| | | AGCAAGCTGACCGTGGACAAGTCCAGGTGGCAGCAGGGC |
| | | AACGTGTTCAGCTGCAGCGTGATGCACGAGGCCCTGCACA |
| | | ACCACTACACCCAGAAGAGCCTGAGCCTGTCCCCCGGCAA |
| | | G |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: 96 | VL | AIRLTQSPSSFSASTGDRVTITCRASESVEYYGTSLMQWYQQ |
| | | KPGKAPKLLIYAASNVESGVPSRFSGSGSGTDFTLTISSLQSE |
| | | DFATYYCQQSRKDPSTFGGGTKVEIK |
| SEQ ID NO: 97 | DNA VL | GCCATCAGACTGACCCAGAGCCCCAGCTCCTTTAGCGCCA |
| | | GCACCGGCGACAGAGTGACCATCACCTGTAGAGCCAGCG |
| | | AGAGCGTGGAATATTACGGCACCAGCCTGATGCAGTGGTA |
| | | TCAGCAGAAGCCCGGCAAGGCCCCCAAGCTGCTGATCTAC |
| | | GCCGCCAGCAATGTGGAAAGCGGCGTGCCCAGCAGATTC |
| | | AGCGGCTCTGGCAGCGGCACCGACTTCACCCTGACAATCA |
| | | GCAGCCTGCAGAGCGAGGACTTCGCCACCTACTACTGCCA |
| | | GCAGAGCCGGAAGGACCCCAGCACATTTGGCGGAGGCAC |
| | | CAAGGTGGAAATCAAG |
| SEQ ID NO: 98 | Light | AIRLTQSPSSFSASTGDRVTITCRASESVEYYGTSLMQWYQQ |
| | Chain | KPGKAPKLLIYAASNVESGVPSRFSGSGSGTDFTLTISSLQSE |
| | | DFATYYCQQSRKDPSTFGGGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: 99 | DNA Light | GCCATCAGACTGACCCAGAGCCCCAGCTCCTTTAGCGCCA |
| | Chain | GCACCGGCGACAGAGTGACCATCACCTGTAGAGCCAGCG |
| | | AGAGCGTGGAATATTACGGCACCAGCCTGATGCAGTGGTA |
| | | TCAGCAGAAGCCCGGCAAGGCCCCCAAGCTGCTGATCTAC |
| | | GCCGCCAGCAATGTGGAAAGCGGCGTGCCCAGCAGATTC |
| | | AGCGGCTCTGGCAGCGGCACCGACTTCACCCTGACAATCA |
| | | GCAGCCTGCAGAGCGAGGACTTCGCCACCTACTACTGCCA |
| | | GCAGAGCCGGAAGGACCCCAGCACATTTGGCGGAGGCAC |
| | | CAAGGTGGAAATCAAGCGTACGGTGGCCGCTCCCAGCGT |
| | | GTTCATCTTCCCCCCCAGCGACGAGCAGCTGAAGAGCGGC |
| | | ACCGCCAGCGTGGTGTGCCTGCTGAACAACTTCTACCCCC |
| | | GGGAGGCCAAGGTGCAGTGGAAGGTGGACAACGCCCTGC |
| | | AGAGCGGCAACAGCCAGGAGAGCGTCACCGAGCAGGACA |
| | | GCAAGGACTCCACCTACAGCCTGAGCAGCACCCTGACCCT |
| | | GAGCAAGGCCGACTACGAGAAGCATAAGGTGTACGCCTGC |
| | | GAGGTGACCCACCAGGGCCTGTCCAGCCCCGTGACCAAG |
| | | AGCTTCAACAGGGGCGAGTGC |
| ABTIM3- | | |
| hum23 | | |
| SEQ ID NO: 3 | HCDR1 | SYNMH |
| (Kabat) | | |
| SEQ ID NO: 4 | HCDR2 | DIYPGNGDTSYNQKFKG |
| (Kabat) | | |
| SEQ ID NO: 5 | HCDR3 | VGGAFPMDY |
| (Kabat) | | |
| SEQ ID NO: 9 | HCDR1 | GYTFTSY |
| (Chothia) | | |
| SEQ ID NO: 10 | HCDR2 | YPGNGD |
| (Chothia) | | |
| SEQ ID NO: 5 | HCDR3 | VGGAFPMDY |
| (Chothia) | | |
| SEQ ID NO: | VH | QVQLVQSGAEVKKPGESLKISCKGSGYTFTSYNMHWVRQMP |
| 100 | | GKGLEWMGDIYPGNGDTSYNQKFKGQVTISADKSISTVYLQW |
| | | SSLKASDTAMYYCARVGGAFPMDYWGQGTTVTVSS |
| SEQ ID NO: | DNA VH | CAGGTGCAATTGGTACAGTCTGGCGCAGAAGTAAAGAAAC |
| 101 | | CAGGAGAGAGTTTGAAAATTTCCTGCAAGGGCAGTGGGTA |
| | | CACATTCACGTCCTACAATATGCACTGGGTGAGACAGATGC |
| | | CAGGCAAGGGCCTGGAGTGGATGGGAGACATATACCCAG |
| | | GCAATGGAGACACAAGCTATAATCAGAAATTCAAAGGACAG |
| | | GTGACGATCTCCGCAGACAAATCCATATCTACGGTCTACCT |
| | | CCAGTGGTCCTCACTTAAAGCCTCCGACACCGCCATGTAC |
| | | TATTGCGCTCGGGTAGGTGGCGCGTTTCCAATGGACTATT |
| | | GGGGCCAAGGGACCACAGTAACCGTCAGCTCA |
| SEQ ID NO: | Heavy | QVQLVQSGAEVKKPGESLKISKGSGYTFTSYNMHWVRQMP |
| 102 | Chain | GKGLEWMGDIYPGNGDTSYNQKFKGQVTISADKSISTVYLQW |
| | | SSLKASDTAMYYCARVGGAFPMDYWGQGTTVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPSN |
| | | TKVDKRVEPKSCDKTHTCPPCPAPELLGGPSVFLFPPKPKDT |
| | | LMISRTPEVTCVVVAVSHEDPEVKFNWYVDGVEVHNAKTKPR |
| | | EEQYNSTYRVVSVLTVLHQDWLNGKEYKCKVSNKALAAPIEK |
| | | TISKAKGQPREPQVYTLPPSREEMTKNQVSLTCLVKGFYPSDI |
| | | AVEWESNGQPENNYKTTPPVLDSDGSFFLYSKLTVDKSRWQ |
| | | QGNVFSCSVMHEALHNHYTQKSLSLSPGK |
| SEQ ID NO: | DNA | CAGGTGCAATTGGTACAGTCTGGCGCAGAAGTAAAGAAAC |
| 103 | Heavy | CAGGAGAGAGTTTGAAAATTTCCTGCAAGGGCAGTGGGTA |
| | Chain | CACATTCACGTCCTACAATATGCACTGGGTGAGACAGATGC |
| | | CAGGCAAGGGCCTGGAGTGGATGGGAGACATATACCCAG |
| | | GCAATGGAGACACAAGCTATAATCAGAAATTCAAAGGACAG |
| | | GTGACGATCTCCGCAGACAAATCCATATCTACGGTCTACCT |
| | | CCAGTGGTCCTCACTTAAAGCCTCCGACACCGCCATGTAC |
| | | TATTGCGCTCGGGTAGGTGGCGCGTTTCCAATGGACTATT |
| | | GGGGCCAAGGGACCACAGTAACCGTCAGCTCAGCTAGCA |
| | | CCAAGGGCCCCAGCGTGTTCCCCCTGGCCCCCAGCAGCA |
| | | AGAGCACCAGCGGCGGCACAGCCGCCCTGGGCTGCCTGG |
| | | TGAAGGACTACTTCCCCGAGCCCGTGACCGTGTCCTGGAA |
| | | CAGCGGAGCCCTGACCTCCGGCGTGCACACCTTCCCCGC |
| | | CGTGCTGCAGAGCAGCGGCCTGTACAGCCTGTCCAGCGT |
| | | GGTGACAGTGCCCAGCAGCAGCCTGGGCACCCAGACCTA |
| | | CATCTGCAACGTGAACCACAAGCCCAGCAACACCAAGGTG |
| | | GACAAGAGAGTGGAGCCCAAGAGCTGCGACAAGACCCAC |
| | | ACCTGCCCCCCCTGCCCAGCCCCAGAGCTGCTGGGCGGA |
| | | CCCTCCGTGTTCCTGTTCCCCCCCAAGCCCAAGGACACCC |
| | | TGATGATCAGCAGGACCCCCGAGGTGACCTGCGTGGTGGT |
| | | GGCCGTGAGCCACGAGGACCCAGAGGTGAAGTTCAACTG |
| | | GTACGTGGACGGCGTGGAGGTGCACAACGCCAAGACCAA |
| | | GCCCAGAGAGGAGCAGTACAACAGCACCTACAGGGTGGT |
| | | GTCCGTGCTGACCGTGCTGCACCAGGACTGGCTGAACGG |
| | | CAAGGAATACAAGTGCAAGGTCTCCAACAAGGCCCTGGCA |
| | | GCCCCCATCGAAAAGACCATCAGCAAGGCCAAGGGCCAG |
| | | CCACGGGAGCCCCAGGTGTACACCCTGCCCCCCTCCCGG |
| | | GAGGAGATGACCAAGAACCAGGTGTCCCTGACCTGTCTGG |
| | | TGAAGGGCTTCTACCCCAGCGACATCGCCGTGGAGTGGGA |
| | | GAGCAACGGCCAGCCCGAGAACAACTACAAGACCACCCCC |
| | | CCAGTGCTGGACAGCGACGGCAGCTTCTTCCTGTACAGCA |
| | | AGCTGACCGTGGACAAGTCCAGGTGGCAGCAGGGCAACG |
| | | TGTTCAGCTGCAGCGTGATGCACGAGGCCCTGCACAACCA |
| | | CTACACCCAGAAGAGCCTGAGCCTGTCCCCCGGCAAG |
| SEQ ID NO: 6 | LCDR1 | RASESVEYYGTSLMQ |
| (Kabat) | | |
| SEQ ID NO: 7 | LCDR2 | AASNVES |
| (Kabat) | | |
| SEQ ID NO: 8 | LCDR3 | QQSRKDPST |
| (Kabat) | | |
| SEQ ID NO: 12 | LCDR1 | SESVEYYGTSL |
| (Chothia) | | |
| SEQ ID NO: 13 | LCDR2 | AAS |
| (Chothia) | | |
| SEQ ID NO: 14 | LCDR3 | SRKDPS |
| (Chothia) | | |
| SEQ ID NO: | VL | AIQLTQSPSSLSASVGDRVTITCRASESVEYYGTSLMQWYQQ |
| 104 | | KPGKAPKLLIYAASNVESGVPSRFSGSGSGTDFTLTISSLQPE |
| | | DFATYFCQQSRKDPSTFGGGTKVEIK |
| SEQ ID NO: | DNA VL | GCAATACAGTTGACACAGAGTCCTTCAAGTTTGTCCGCTTC |
| 105 | | CGTTGGCGACCGAGTGACAATCACCTGTAGAGCATCCGAG |
| | | TCAGTGGAGTATTATGGCACTAGCCTGATGCAGTGGTATCA |
| | | GCAAAAGCCAGGGAAAGCCCCAAAGCTGCTGATATATGCC |
| | | GCGAGTAACGTCGAGTCAGGGGTGCCATCAAGATTCTCCG |
| | | GTTCCGGGTCCGGAACCGACTTCACACTGACCATCTCTTC |
| | | CCTTCAGCCAGAGGACTTCGCTACGTACTTTTGCCAGCAGT |
| | | CACGGAAAGATCCCTCTACTTTCGGAGGTGGGACAAAAGT |
| | | CGAAATTAAA |
| SEQ ID NO: | Light | AIQLTQSPSSLSASVGDRVTITCRASESVEYYGTSLMQWYQQ |
| 106 | Chain | KPGKAPKLLIYAASNVESGVPSRFSGSGSGTDFTLTISSLQPE |
| | | DFATYFCQQSRKDPSTFGGGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| SEQ ID NO: | DNA Light | GCAATACAGTTGACACAGAGTCCTTCAAGTTTGTCCGCTTC |
| 107 | Chain | CGTTGGCGACCGAGTGACAATCACCTGTAGAGCATCCGAG |
| | | TCAGTGGAGTATTATGGCACTAGCCTGATGCAGTGGTATCA |
| | | GCAAAAGCCAGGGAAAGCCCCAAAGCTGCTGATATATGCC |
| | | GCGAGTAACGTCGAGTCAGGGGTGCCATCAAGATTCTCCG |
| | | GTTCCGGGTCCGGAACCGACTTCACACTGACCATCTCTTC |
| | | CCTTCAGCCAGAGGACTTCGCTACGTACTTTTGCCAGCAGT |
| | | CACGGAAAGATCCCTCTACTTTCGGAGGTGGGACAAAAGT |
| | | CGAAATTAAACGTACGGTGGCCGCTCCCAGCGTGTTCATC |
| | | TTCCCCCCCAGCGACGAGCAGCTGAAGAGCGGCACCGCC |
| | | AGCGTGGTGTGCCTGCTGAACAACTTCTACCCCCGGGAGG |
| | | CCAAGGTGCAGTGGAAGGTGGACAACGCCCTGCAGAGCG |
| | | GCAACAGCCAGGAGAGCGTCACCGAGCAGGACAGCAAGG |
| | | ACTCCACCTACAGCCTGAGCAGCACCCTGACCCTGAGCAA |
| | | GGCCGACTACGAGAAGCATAAGGTGTACGCCTGCGAGGT |
| | | GACCCACCAGGGCCTGTCCAGCCCCGTGACCAAGAGCTTC |
| | | AACAGGGGCGAGTGC |

TABLE 5
Constant region amino acid sequences of human IgG heavy chains and
human kappa light chain

| | |
|---|---|
| HC | IgG4 (S228P) mutant constant region amino acid sequence (EU Numbering) |
| | ASTKGPSVFP LAPCSRSTSE STAALGCLVK DYFPEPVTVS WNSGALTSGV |
| | HTFPAVLQSS GLYSLSSVVT VPSSSLGTKT YTCNVDHKPS NTKVDKRVES |
| | KYGPPCPPCP APEFLGGPSV FLFPPKPKDT LMISRTPEVT CVVVDVSQED |
| | PEVQFNWYVD GVEVHNAKTK PREEQFNSTY RVVSVLTVLH QDWLNGKEYK |
| | CKVSNKGLPS SIEKTISKAK GQPREPQVYT LPPSQEEMTK NQVSLTCLVK |
| | GFYPSDIAVE WESNGQPENN YKTTPPVLDS DGSFFLYSRL TVDKSRWQEG |
| | NVFSCSVMHE ALHNHYTQKS LSLSLGK (SEQ ID NO: 108) |
| LC | Human kappa constant region amino acid sequence |
| | RTVAAPSVFI FPPSDEQLKS GTASVVCLLN NFYPREAKVQ WKVDNALQSG |
| | NSQESVTEQD SKDSTYSLSS TLTLSKADYE KHKVYACEVT HQGLSSPVTK |
| | SFNRGEC (SEQ ID NO: 109) |
| HC | IgG4 (S228P) mutant constant region amino acid sequence lacing C-terminal lysine |
| | (K) (EU Numbering) |
| | ASTKGPSVFP LAPCSRSTSE STAALGCLVK DYFPEPVTVS WNSGALTSGV |
| | HTFPAVLQSS GLYSLSSVVT VPSSSLGTKT YTCNVDHKPS NTKVDKRVES |
| | KYGPPCPPCP APEFLGGPSV FLFPPKPKDT LMISRTPEVT CVVVDVSQED |
| | PEVQFNWYVD GVEVHNAKTK PREEQFNSTY RVVSVLTVLH QDWLNGKEYK |
| | CKVSNKGLPS SIEKTISKAK GQPREPQVYT LPPSQEEMTK NQVSLTCLVK |
| | GFYPSDIAVE WESNGQPENN YKTTPPVLDS DGSFFLYSRL TVDKSRWQEG |
| | NVFSCSVMHE ALHNHYTQKS LSLSLG (SEQ ID NO: 110) |
| HC | IgG1 wild type |
| | ASTKGPSVFP LAPSSKSTSG GTAALGCLVK DYFPEPVTVS WNSGALTSGV |
| | HTFPAVLQSS GLYSLSSVVT VPSSSLGTQT YICNVNHKPS NTKVDKRVEP |
| | KSCDKTHTCP PCPAPELLGG PSVFLFPPKP KDTLMISRTP EVTCVVVDVS |
| | HEDPEVKFNW YVDGVEVHNA KTKPREEQYN STYRVVSVLT VLHQDWLNGK |
| | EYKCKVSNKA LPAPIEKTIS KAKGQPREPQ VYTLPPSREE MTKNQVSLTC |
| | LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 111) |
| HC | IgG1 (N297A) mutant constant region amino acid sequence (EU Numbering) |
| | ASTKGPSVFP LAPSSKSTSG GTAALGCLVK DYFPEPVTVS WNSGALTSGV |
| | HTFPAVLQSS GLYSLSSVVT VPSSSLGTQT YICNVNHKPS NTKVDKRVEP |
| | KSCDKTHTCP PCPAPELLGG PSVFLFPPKP KDTLMISRTP EVTCVVVDVS |
| | HEDPEVKFNW YVDGVEVHNA KTKPREEQYA STYRVVSVLT VLHQDWLNGK |
| | EYKCKVSNKA LPAPIEKTIS KAKGQPREPQ VYTLPPSREE MTKNQVSLTC |
| | LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 112) |
| HC | IgG1 (D265A, P329A) mutant constant region amino acid sequence (EU Numbering) |
| | ASTKGPSVFP LAPSSKSTSG GTAALGCLVK DYFPEPVTVS WNSGALTSGV |
| | HTFPAVLQSS GLYSLSSVVT VPSSSLGTQT YICNVNHKPS NTKVDKRVEP |
| | KSCDKTHTCP PCPAPELLGG PSVFLFPPKP KDTLMISRTP EVTCVVVAVS |
| | HEDPEVKFNW YVDGVEVHNA KTKPREEQYN STYRVVSVLT VLHQDWLNGK |
| | EYKCKVSNKA LAAPIEKTIS KAKGQPREPQ VYTLPPSREE MTKNQVSLTC |
| | LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 113) |
| HC | IgG1 (L234A, L235A) mutant constant region amino acid sequence (EU Numbering) |
| | ASTKGPSVFP LAPSSKSTSG GTAALGCLVK DYFPEPVTVS WNSGALTSGV |
| | HTFPAVLQSS GLYSLSSVVT VPSSSLGTQT YICNVNHKPS NTKVDKRVEP |
| | KSCDKTHTCP PCPAPEAAGG PSVFLFPPKP KDTLMISRTP EVTCVVVDVS |
| | HEDPEVKFNW YVDGVEVHNA KTKPREEQYN STYRVVSVLT VLHQDWLNGK |
| | EYKCKVSNKA LPAPIEKTIS KAKGQPREPQ VYTLPPSREE MTKNQVSLTC |
| | LVKGFYPSDI AVEWESNGQP ENNYKTTPPV LDSDGSFFLY SKLTVDKSRW |
| | QQGNVFSCSV MHEALHNHYT QKSLSLSPGK (SEQ ID NO: 114) |

In some embodiments, the anti-TIM3 antibody is MBG453.

Other Exemplary TIM-3 Inhibitors

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of TSR-022. In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121, e.g., as disclosed in Table 6. APE5137, APE5121, and other anti-TIM-3 antibodies are disclosed in WO 2016/161270, incorporated by reference in its entirety.

In one embodiment, the anti-TIM-3 antibody molecule is the antibody clone F38-2E2. In one embodiment, the anti-TIM-3 antibody molecule comprises one or more of the CDR sequences (or collectively all of the CDR sequences), the heavy chain or light chain variable region sequence, or the heavy chain or light chain sequence of F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In one embodiment, the anti-TIM-3 antibody is an antibody that competes for binding with, and/or binds to the same epitope on TIM-3 as, one of the anti-TIM-3 antibodies described herein.

TABLE 6
Amino acid sequences of other exemplary anti-TIM-3 antibody molecules
APE5137

| | | |
|---|---|---|
| SEQ ID NO: 830 | VH | EVQLLESGGGLVQPGGSLRLSCAAASGFTFSSYDMSWVRQAPGK |
| | | GLDWVSTISGGGTYTYYQDSVKGRFTISRDNSKNTLYLQMNSLRAE |
| | | DTAVYYCASMDYWGQGTTVTVSSA |
| SEQ ID NO: 831 | VL | DIQMTQSPSSLSASVGDRVTITCRASQSIRRYLNWYHQKPGKAPKL |
| | | LIYGASTLQSGVPSRFSGSGSGTDFTLTISSLQPEDFAVYYCQQSH |
| | | SAPLTFGGGTKVEIKIR |

APE5121

| | | |
|---|---|---|
| SEQ ID NO: 832 | VH | EVQLVESGGGLVQPGGSLRLYCVASGFTFSGSYAMSWVRQAPGK |
| | | GLEWVSAISGSGGSTYYADSVKGRFTISRDNSKNTLYLQMNSLRAE |
| | | DTAVYYCAKKYYVGPADYWGQGTLVTVSSG |
| SEQ ID NO: 833 | VL | DIVMTQSPDSLAVSLGERATINCKSSQSVLYSSNNKNYLAWYQHKP |
| | | GQPPKLLIYWASTRESGVPDRFSGSGSGTDFTLTISSLQAEDVAVY |
| | | YCQQYYSSPLTFGGGTKIEVK |

As used herein, “ruxolitinib” is the JAK1/JAK2 inhibitor (R)-3-(4-(7H-pyrrolo[2,3-d]pyrimidin-4-yl)-1H-pyrazol-1-yl)-3-cyclopentylpropanenitrile, also named 3(R)-Cyclopentyl-3-[4-(7H-pyrrolo[2,3-d]pyrimidin-4-yl)-1H-pyrazol-1-yl]propanenitrile, of formula:

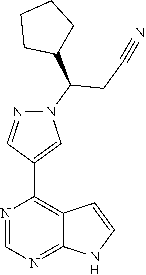

which can be prepared, for example, as described in WO2007/070514, which is incorporated herein by reference. As used herein, “ruxolitinib” refers to the free form, and any reference to “a pharmaceutically acceptable salt thereof” refers to “a pharmaceutically acceptable acid addition salt thereof”, in particular ruxolitinib phosphate, which can be prepared, for example, as described in WO2008/157208, which is incorporated herein by reference. Ruxolitinib is approved for the treatment of intermediate to high-risk myelofibrosis under the tradename Jakafi®/Jakavi®.

Ruxolitinib, or pharmaceutically acceptable salt thereof, in particular ruxolitinib phosphate, can be in a unit dosage form (e.g. tablet), which is administered orally.

In one embodiment, “ruxolitinib” is also intended to represent isotopically labeled forms. Isotopically labeled compounds have structures depicted by the formula above except that one or more atoms are replaced by an atom having a selected atomic mass or mass number. Isotopes that can be incorporated into ruxolitinib, for example, isotopes of hydrogen, namely the compound of formula:

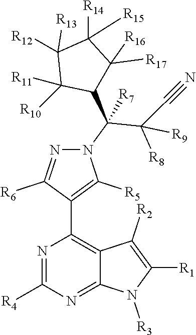

wherein each R₁, R₂, R₃, R₄, R₅, R₆, R₇, R₈, R₉, R₁₀, R₁₁, R₁₂, R₁₃, R₁₄, R₁₅, R₁₆ and R₁₇ is independently selected from H or deuterium; provided that there is at least one deuterium present in the compound. In other embodiments there are multiple deuterium atoms present in the compound. Suitable compounds are disclosed in U.S. Pat. No. 9,249,149 B2, which is hereby incorporated in its entirety.

In one preferred embodiment, a deuterated ruxolitinib is selected from the group consisting of

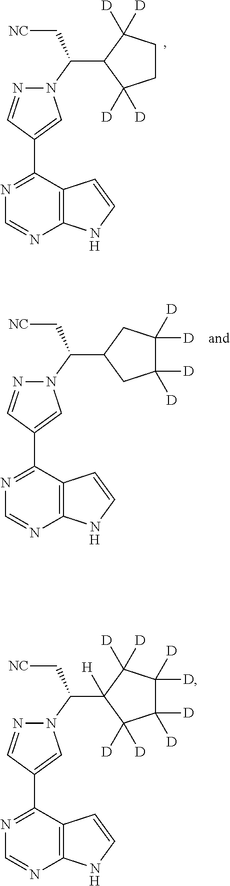

or a pharmaceutically acceptable salt of any of the foregoing.

In a preferred embodiment, a deuterated ruxolitinib is

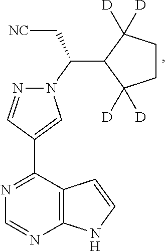

or a pharmaceutically acceptable salt thereof.

As used herein, “itacitinib” refers to the JAK1/JAK2 inhibitor 2-(3-(4-(7H-pyrrolo(2,3-d)pyrimidin-4-yl)-1H-pyrazol-1-yl)-1-(1-(3-fluoro-2-(trifluoromethyl)isonicotinoyl)piperidin-4-yl)azetidin-3-yl)acetonitrile, also named 2-[1-[1-[3-fluoro-2-(trifluoromethyl)pyridine-4-carbonyl]piperidin-4-yl]-3-[4-(7H-pyrrolo[2,3-d]pyrimidin-4-yl)pyrazol-1-yl]azetidin-3-yl]acetonitrile of formula

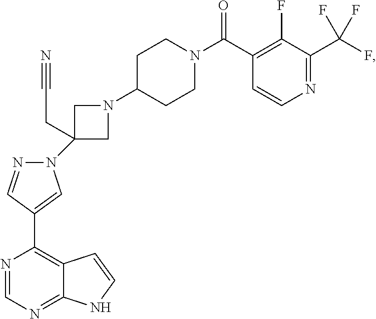 [SMILES: N#CCC1(n2cc(-c3ncnc4[nH]ccc34)cn2)CN(C2CCN(C(=O)c3ccnc(C(F)(F)F)c3F)CC2)C1]

which can be prepared, for example, as described in WO2011/112662, which is incorporated herein by reference. As used herein, “itacitinib” refers to the free form, and any reference to “a pharmaceutically acceptable salt thereof” refers to “a pharmaceutically acceptable acid addition salt thereof”, in particular itacitinib adipate.

### Treatment of Myelofibrosis

In one aspect the present invention provide an anti-TIM-3 antibody molecule, alone or in combination with a JAK inhibitor, (e.g., ruxolitinib) or a pharmaceutical acceptable salt thereof, for use in the treatment of Philadelphia-chromosome negative myeloproliferative neoplasms.

In one further aspect the present invention provides an anti-TIM-3 antibody molecule, for use in the treatment of myelofibrosis (MF) in a patient. Alternatively, in one aspect the present invention provides an an anti-TIM-3 antibody molecule for use in the manufacture of a medicament for the treatment of myelofibrosis (MF) in a patient. Alternatively, in one aspect the present invention provides a method of treating myelofibrosis (MF) in a patient comprising the step of administering therapeutically effective amount of an anti-TIM-3 antibody molecule, to said patient.

Myelofibrosis comprises primary myelofibrosis (PMF), post-essential thrombocythemia myelofibrosis (PET-MF) and post-polycythemia vera myelofibrosis (PPV-MF). Suitably, myelofibrosis is PMF.

The term “primary myelofibrosis” (PMF), as used herein, is defined with reference to “The 2016 revision to the World Health Organization (WHO) classification of myeloid neoplasms and acute leukemia”, as published in Blood, 2016, 127:2391-2405. Primary myelofibrosis encompasses prefibrotic/early primary myelofibrosis (prePMF) and overt primary myelofibrosis (overt PMF). Diagnosis of prePMF requires meeting the following 3 major criteria, and at least 1 minor criterion according to the 2016 WHO classification for prePMF in table A:

TABLE A
Criteria for diagnosis of prePMF

Major criteria (prePMF)
1. Megakaryocytic proliferation and atypia, without reticulin fibrosis > grade 1, accompanied by increased
age-adjusted BM cellularity, granulocytic proliferation, and often decreased erythropoiesis
2. Not meeting the WHO criteria for BCR-ABL1⁺CML, PV, ET, myelodysplastic syndromes, or other
myeloid neoplasms
3. Presence of JAK2, CALR, or MPL mutation or in the absence of these mutations, presence of another
clonal marker (e.g., ASXL1, EZH2, TET2, IDH1/IDH2, SRSF2, SF3B1) are of help in determining the
clonal nature of the disease or absence of minor reactive bone marrow (BM) reticulin fibrosis (Minor
(grade 1) reticulin fibrosis secondary to infection, autoimmune disorder or other chronic inflammatory
conditions, hairy cell leukemia or other lymphoid neoplasm, metastatic malignancy, or toxic (chronic)
myelopathies)
Minor criteria (prePMF)
Presence of at least 1 of the following, confirmed in 2 consecutive determinations:
a. Anemia not attributed to a comorbid condition
b. Leukocytosis ≥11*10⁹/L
c. Palpable splenomegaly
d. LDH increased to above upper normal limit of institutional reference range

Diagnosis of overt PMF requires meeting the following 3 major criteria, and at least 1 minor criterion according to the 2016 WHO classification for overt PMF in table B:

TABLE B
Criteria for diagnosis of overt PMF

Major criteria (overt PMF)
1. Presence of megakaryocytic proliferation and atypia, accompanied by either reticulin and/or collagen
fibrosis grades 2 or 3
2. Not meeting WHO criteria for ET, PV, BCR-ABL1⁺CML, myelodysplastic syndromes, or other myeloid
neoplasms
3. Presence of JAK2, CALR, or MPL mutation or in the absence of these mutations, presence of another
clonal marker (e.g., ASXL1, EZH2, TET2, IDH1/IDH2, SRSF2, SF3B1) or absence of reactive
myelofibrosis (BM fibrosis secondary to infection, autoimmune disorder, or other chronic inflammatory
conditions, hairy cell leukemia or other lymphoid neoplasm, metastatic malignancy, or toxic (chronic)
myelopathies)
Minor criteria (overt PMF)
Presence of at least 1 of the following, confirmed in 2 consecutive determinations:
a. Anemia not attributed to a comorbid condition
b. Leukocytosis ≥11*10⁹/L
c. Palpable splenomegaly
d. LDH increased to above upper normal limit of institutional reference range
e. Leukoerythroblastosis

The term “bone marrow fibrosis”, as used herein, refers to bone marrow fibrosis graded according to the 2005 European consensus grading system (Thiele et. al., Haematologica, 2005, 90(8), 1128-1132, in particular as defined in Table 3 and FIG. 1 of page 1130 therein), such as:
“fibrosis grade 0”: scattered linear reticulin with no intersections (cross-overs) corresponding to normal bone marrow;
“fibrosis grade 1”: loose network of reticulin with many intersections, especially in perivascular areas;
“fibrosis grade 2”: diffuse and dense increase in reticulin with extensive intersections, occasionally with only focal bundles of collagen and/or focal osteosclerosis;
“fibrosis grade 3”: diffuse and dense increase in reticulin with extensive intersections with coarse bundles of collagen, often associated with significant osteosclerosis;
wherein the grading (i.e. grading of fiber density and quality) is made on the basis of bone marrow biopsy specimen assessment.

The term “essential thrombocythemia” (ET), as used herein, is defined with reference to “The 2016 revision to the World Health Organization (WHO) classification of myeloid neoplasms and acute leukemia”, as published in Blood, 2016, 127:2391-2405. The term “post-essential thrombocythemia myelofibrosis” (PET-MF), as used herein, refers to MF secondary to ET (i.e. MF arising as a progression of ET), wherein ET is as defined herein above. According to the IWG-MRT criteria (Barosi G et al, Leukemia (2008) 22, 437-438), criteria for diagnosing post-essential thrombocythemia myelofibrosis are:

TABLE C
Criteria for diagnosis of post-essential thrombocythemia myelofibrosis

Required criteria:
1. Documentation of a previous diagnosis of essential thrombocythemia as defined by the WHO criteria
2. Bone marrow fibrosis grade 2-3
Additional criteria (two are required):
1. Anemia and a ≥2 mg/ml decrease from baseline hemoglobin level
2. A leukoerythroblastic peripheral blood picture
3. Increasing splenomegaly defined as either an increase in palpable splenomegaly of ≥5 cm (distance of
the tip of the spleen from the left costal margin) or the appearance of a newly palpable splenomegaly
4. Increased lactate dehydrogenase (LDH) (above reference level)
5. Development of ≥1 of three constitutional symptoms: >10% weight loss in 6 months, night sweats,
unexplained fever (>37.5° C.)

The term “polycythemia vera” (PV), as used herein, is defined with reference to “The 2016 revision to the World Health Organization (WHO) classification of myeloid neoplasms and acute leukemia”, as published in Blood, 2016, 127:2391-2405. The term “post-polycythemia myelofibrosis” (PPV-MF), as used herein, refers to MF secondary to PV (i.e. MF arising as a progression of PV). According to the IWG-MRT criteria (Barosi G et al, Leukemia (2008) 22, 437-438), criteria for diagnosing post-polycythemia myelofibrosis are:

TABLE D
Criteria for diagnosis of post-polycythemia myelofibrosis

Required criteria:
1. Documentation of a previous diagnosis of polycythemia vera as defined by the WHO criteria
2. Bone marrow fibrosis grade 2-3 (on 0-3 scale)
Additional criteria (two are required):
1. Anemia or sustained loss of requirement of either phlebotomy (in the absence of cytoreductive
therapy) or cytoreductive treatment for erythrocytosis
2. A leukoerythroblastic peripheral blood picture
3. Increasing splenomegaly defined as either an increase in palpable splenomegaly of ≥5 cm (distance of
the tip of the spleen from the left costal margin) or the appearance of a newly palpable splenomegaly
4. Development of ≥1 of three constitutional symptoms: >10% weight loss in 6 months, night sweats,
unexplained fever (>37.5° C.)

As used herein, the following response criteria as defined by the International Working Group-Myeloproliferative Neoplasms Research and Treatment (IWG-MRT) and the European Leukemia Net (ELN) response criteria for MF (Tefferi et al, Blood 2013 122:1395-1398, which is incorporated by reference in its entirety) are used herein:

TABLE E
International Working Group-Myeloproliferative Neoplasms Research and Treatment
(IWG-MRT) and the European Leukemia Net (ELN) response criteria for myelofibrosis

| Response | Required criteria (for all response categories, benefit must last for ≥12 weeks to |
|---|---|
| categories | qualify as a response) |
| Complete | Bone marrow:* Age-adjusted normocellularity; <5% blasts; ≤grade 1 MF^{†} and |
| response | Peripheral blood: Hemoglobin ≥100 g/L and <UNL; neutrophil count ≥1 × 10⁹/L |
| (CR) | and <UNL; |
| | Platelet count ≥100 × 10⁹/L and <UNL; <2% immature myeloid cells^{‡} and |
| | Clinical: Resolution of disease symptoms; spleen and liver not palpable; no evidence |
| | of EMH |
| Partial | Peripheral blood: Hemoglobin ≥100 g/L and <UNL; neutrophil count ≥1 × 10⁹/L |
| response | and <UNL; platelet count ≥100 × 10⁹/L and <UNL; <2% immature myeloid cells^{‡} and |
| (PR) | Clinical: Resolution of disease symptoms; spleen and liver not palpable; no evidence |
| | of EMH or |
| | Bone marrow:* Age-adjusted normocellularity; <5% blasts; ≤grade 1 MF^{†}, and |
| | peripheral blood: Hemoglobin ≥85 but <100 g/L and <UNL; neutrophil count ≥1 × |
| | 10⁹/L and <UNL; platelet count ≥50, but <100 × 10⁹/L and <UNL; <2% immature |
| | myeloid cells^{‡} and |
| | Clinical: Resolution of disease symptoms; spleen and liver not palpable; no evidence |
| | of EMH |
| Clinical | The achievement of anemia, spleen or symptoms response without progressive |
| improvement (CI) | disease or increase in severity of anemia, thrombocytopenia, or neutropenia^{§} |
| Anemia | Transfusion-independent patients: a ≥20 g/L increase in hemoglobin level^{∥} |
| response | Transfusion-dependent patients: becoming transfusion-independent^{¶} |
| Spleen | A baseline splenomegaly that is palpable at 5-10 cm, below the LCM, becomes not |
| response^{#} | palpable** or |
| | A baseline splenomegaly that is palpable at >10 cm, below the LCM, decreases by ≥50%** |
| | A baseline splenomegaly that is palpable at <5 cm, below the LCM, is not eligible for |
| | spleen response |
| | A spleen response requires confirmation by MRI or computed tomography showing ≥35% |
| | spleen volume reduction |
| Symptoms | A ≥50% reduction in the MPN-SAF TSS^{††} |
| response | |

EMH, extramedullary hematopoiesis (no evidence of EMH implies the absence of pathology- or imaging study-proven nonhepatosplenic EMH); LCM, left costal margin; UNL, upper normal limit.
*Baseline and posttreatment bone marrow slides are to be interpreted at one sitting by a central review process.
^{†}Grading of MF is according to the European classification: Thiele et al. European consensus on grading bone marrow fibrosis and assessment of cellularity. Haematologica. 2005; 90: 1128.
^{‡}Immature myeloid cells constitute blasts + promyelocytes + myelocytes + metamyelocytes + nucleated red blood cells. In splenectomized patients, <5% immature myeloid cells is allowed.
^{§}Increase in severity of anemia constitutes the occurrence of new transfusion dependency or a ≥20 g/L decrease in hemoglobin level from pretreatment baseline that lasts for at least 12 weeks. Increase in severity of thrombocytopenia or neutropenia is defines as a 2-grade decline, from pretreatment baseline, in platelet count or absolute neutrophil count, according to the Common Terminology Criteria for Adverse Events (CTCAE) version 4.0. In addition, assignment to CI requires a minimum platelet count of ≥25 000 × 10(9)/L and absolute neutrophil count of ≥0.5 × 10(9)/L.
^{∥}Applicable only to patients with baseline hemoglobin of <100 g/L. In patients not meeting the strict criteria for transfusison dependency at the time of treatment initiation, but have received transfusions within the previous month, the pre-transfusion hemoglobin level should be used as the baseline.
^{¶}Transfusion dependency is defines as transfusions of at least 6 units of packed red blood cells (PRBC), in the 12 weeks prior to start of treatment initiation, for a hemoglobin level of <85 g/L, in the absence of bleeding or treatment-induced anemia. In addition, the most recent transfusion episode must have occurred in the 28 days prior to start of treatment initiation. Response in transfusion-dependent patients requires absence of any PRBC transfusions during any consecutive “rolling” 12-week interval during the treatment phase, capped by a hemoglobin level of ≥85 g/L.
^{#}In splenectomized patients, palpable hepatomegaly is substituted with the same measurement strategy.
**Spleen or liver responses must be confirmed by imaging studies where a ≥35% reduction in spleen volume, as assessed by MRI or CT, is required. Furthermore, a ≥35% volume reduction in the spleen or liver, by MRI or CT, constitutes a response regardless of what is reported with physical examination.
^{††}Symptoms are evaluated by the MPN-SAF TSS. The MPN-SAF TSS is assessed by the patients themselves and this includes fatigue, concentration, early satiety, inactivity, night sweats, itching, bone pain, abdominal discomfort, weight loss, and fevers. Scoring is from 0 (absent/as good as it can be) to 10 (worst imaginable/as bad as it can be) for each item. The MPN-SAF TSS is the summation of all the individual scores (0-100 scale). Symptoms response requires ≥50% reduction in the MPN-SAF TSS.

In one embodiment the present invention provides an anti-TIM-3 antibody molecule, alone or in combination with a JAK inhibitor, suitably ruxolitinib or a pharmaceutically acceptable salt thereof, for use in the treatment of myelofibrosis, especially primary MF, wherein the patient achieves complete response to the treatment according to the criteria in Table 5.

In one embodiment the present invention provides an anti-TIM-3 antibody molecule, alone or in combination with a JAK inhibitor, suitably ruxolitinib or a pharmaceutically acceptable salt thereof, for use in the treatment of myelofibrosis, especially primary MF, wherein the patient achieves partial response to the treatment according to the criteria in Table 5.

Among patients, myelofibrosis frequently causes shortened survival due to disease transformation to acute leukemia, progression without acute transformation, cardiovascular complications or thrombosis, infection or portal hypertension. It is one of the aims of the present invention to improve the median survival of myelofibrosis patients.

As used herein, the term “median survival time” refers to the time of diagnosis or from the time of initiation of treatment according to the present invention that half of the patients in a group of patients diagnosed with the disease are still alive compared to patients receiving best available treatment or compared to patients receiving placebo and wherein patients belong to the same risk group of myelofibrosis, for example as described by Gangat et al (J Clin Oncol. 2011 Feb. 1; 29(4):392-397), which is hereby incorporated by reference in its entirety.

Accordingly, in one embodiment the present invention provides an anti-TIM-3 antibody molecule, alone or in combination with a JAK inhibitor, suitably ruxolitinib or a pharmaceutically acceptable salt thereof, for use in the treatment of myelofibrosis, especially primary MF, wherein median survival time is increased by at least 3 months in the group of high risk MF patients or by at least six months, preferably by at least 12 months in the group of medium risk MF patients.

As used herein, the term “subject” refers to a human being.

The term “treat”, “treating”, “treatment” or “therapy”, as used herein, means obtaining beneficial or desired results, for example, clinical results. Beneficial or desired results can include, but are not limited to, alleviation of one or more symptoms, as defined herein. One aspect of the treatment is, for example, that said treatment should have a minimal adverse effect on the patient, e.g. the agent used should have a high level of safety, for example without producing the side effects of a previously known therapy. The term “alleviation”, for example in reference to a symptom of a condition, as used herein, refers to reducing at least one of the frequency and amplitude of a symptom of a condition in a patient.

As used herein, the term “newly diagnosed” refers to diagnosis of the disorder, e.g. myelofibrosis and said patient has not received any treatment. In one embodiment the present invention provides an anti-TIM-3 antibody molecule, alone or in combination with a JAK inhibitor, suitably ruxolitinib or a pharmaceutically acceptable salt thereof, for use in the treatment of a newly diagnosed myelofibrosis patient

The term “triple-negative myelofibrosis patient”, as used herein, refers to a patient who lacks JAK2, CALR and MPL mutations. In one embodiment the present invention provides an anti-TIM-3 antibody molecule, alone or in combination with a JAK inhibitor, suitably ruxolitinib or a pharmaceutically acceptable salt thereof, for use in the treatment of triple-negative myelofibrosis patient.

The term “best available therapy”, as used herein, refers to any commercially available agent approved prior to March 2018 for the treatment of PMF, PET-MF or PPV-MF, as monotherapy, or in combination. Exemplary agents include, but are not limited to ruxolitinib or a pharmaceutically acceptable salt thereof, antineoplastic agents (e.g., hydroxyurea, anagrelide), glucocorticoids (e.g., prednisone/prednisolone, methylprednisolone), antianemia preparations (e.g., epoetin-alpha), immunomodulatory agents (e.g., thalidomide, lenalidomide), purine analogs (e.g., mercaptopurine, thioguanine), antigonadotropins (e.g., danazol), interferons (e.g., PEG-interferon-alpha 2a, interferon-alpha), nitrogen mustard analogs (e.g. melphalan), pyrimidine analogs (e.g., cytarabine).

The term “splenomegaly”, as used herein, refers to a palpably enlarged spleen (e.g. a spleen is palpable at ≥5 cm below the left coastal margin) or to an enlarged spleen as detected by an imaging test (e.g. a computed tomography (CT) scan, MRI, X-rays or ultrasound), wherein the term “enlarged spleen” refers to a spleen greater in size than normal (e.g., median normal spleen volume of 200 cm³).

The term “treatment of splenomegaly”, as used herein, refers to “improvement of splenomegaly”, which means a decrease in splenomegaly, for example a reduction in spleen volume, as defined by the International Working Group-Myeloproliferative Neoplasms Research and Treatment (IWG-MRT) and the European Leukemia Net (ELN) response criteria for MF in Table 5. In one embodiment, the invention may provide the use of an anti-TIM-3 antibody molecule or a pharmaceutical acceptable salt thereof, alone or in combination with ruxolitinib or a pharmaceutically acceptable salt thereof for treatment of myelofibrosis, particularly for the treatment of splenomegaly associated with myelofibrosis, resulting in, for example, ≥20%, ≥25%, ≥30% or ≥35% reduction in spleen volume as measured by magnetic resonance imaging (MRI) or computed tomography (CT) from pre-treatment baseline to, for example, week 24 or week 48.

The term “hepatomegaly”, as used herein, refers to a palpably enlarged liver or to an enlarged liver as detected by an imaging test (e.g. a computed tomography (CT) scan), wherein the term “enlarged liver” refers to a liver greater in size than normal (e.g., median normal liver volume of approximately 1500 cm³).

The term “treatment of hepatomegaly”, as used herein, refers to “improvement of hepatomegaly”, which means a decrease in hepatomegaly, for example a reduction in hepatomegaly, as defined according to the International Working Group-Myeloproliferative Neoplasms Research and Treatment (IWG-MRT) and the European Leukemia Net (ELN) response criteria for MF in the preceding table. Accordingly, in one embodiment the present invention provides the use of an anti-TIM-3 antibody molecule, alone or in combination with ruxolitinib or a pharmaceutically acceptable salt thereof for treatment of myelofibrosis, particularly for the treatment of hepatomegaly associated with myelofibrosis, resulting in, for example, ≥20%, ≥25%, ≥30% or ≥35% reduction in liver volume as measured by magnetic resonance imaging (MRI) or computed tomography (CT) from pre-treatment baseline to, for example, week 24 or week 48.

The term “thrombocytopenia”, as used herein, refers to a platelet count, in blood specimen laboratory test, lower than normal. The term “severity of thrombocytopenia”, as used herein, refers, for example, to specific grade 1-4 of thrombocytopenia according to CTCAE (version 4.03).

The term “treatment of thrombocytopenia”, as used herein, refers to “stabilizing thrombocytopenia” or “improving thrombocytopenia”, in comparison to the pre-treatment situation or in comparison to best available therapy or to placebo control. The term “stabilizing thrombocytopenia” refers, for example, to prevent an increase in the severity of thrombocytopenia, namely the platelet count remains stable. The term “improving thrombocytopenia” refers to alleviation of the severity of thrombocytopenia, namely increasing blood platelet count. In one embodiment, the invention provides an anti-TIM-3 antibody molecule, alone or in combination with ruxolitinib or a pharmaceutically acceptable salt thereof, for use in the treatment of myelofibrosis, particularly for the treatment of thrombocytopenia associated with myelofibrosis, resulting in stabilizing thrombocytopenia or improving thrombocytopenia from pre-treatment baseline to, for example, week 24 or week 48 of treatment.

The term “neutropenia”, as used herein, refers to an absolute neutrophil count (ANC), in blood specimen laboratory test, lower than normal value. The term “severity of neutropenia”, as used herein, refers, for example, to specific grade 1-4 of neutropenia according to CTCAE (version 4.03).

The term “treatment of neutropenia”, as used herein, refers to “stabilizing neutropenia” or “improving neutropenia”, for example, in comparison to the pre-treatment situation or in comparison to best available therapy or to placebo control. The term “stabilizing neutropenia” refers, for example, to prevent an increase in the severity of neutropenia. The term “improving neutropenia” refers, for example, to a decrease in the severity of neutropenia. In one embodiment, the invention provides an anti-TIM-3 antibody molecule, with ruxolitinib or a pharmaceutically acceptable salt thereof, for use in the treatment of myelofibrosis, particularly for the treatment of neutropenia associated with myelofibrosis, resulting in stabilizing neutropenia or improving neutropenia from pre-treatment baseline to, for example, week 24 or week 48 of treatment.

The term “anemia”, as used herein, refers to hemoglobin level, in blood specimen laboratory test, of less than 13.5 gram/100 ml in men and hemoglobin level of less than 12.0 gram/100 ml in women. The term “severity of anemia”, as used herein, refers, for example, to specific grade 1-4 of anemia according to CTCAE (version 4.03)].

The term “treatment of anemia”, as used herein, refers to “stabilizing anemia” or “improving anemia”, for example, in comparison to the pre-treatment situation or in comparison to best available therapy or to placebo control. The term “stabilizing anemia” refers, for example, to prevent an increase in the severity of anemia (e.g. preventing that a “transfusion-independent” patient becomes a “transfusion-dependent” patient or preventing anemia grade 2 becomes anemia grade 3). The term “improving anemia” refers to a decrease in the severity of anemia or an improvement in hemoglobin level. In one embodiment, the invention may provide the use of an anti-TIM-3 antibody molecule, alone or in combination with ruxolitinib or a pharmaceutically acceptable salt thereof, for treatment of myelofibrosis, particularly for the treatment of anemia associated with myelofibrosis, resulting in stabilizing anemia or improving anemia from pre-treatment baseline to, for example, week 24 or week 48 of treatment.

The term “treatment of bone marrow fibrosis associated with MF”, as used herein, means “stabilizing bone marrow fibrosis” or “improving bone marrow fibrosis”, for example, in comparison to the pre-treatment situation or in comparison to best available therapy or to placebo control. The term “stabilizing bone marrow fibrosis” refers, for example, to prevent increase in severity of bone marrow fibrosis. The term “improving bone marrow fibrosis” refers to a decrease in severity of bone marrow fibrosis, for example, from pre-treatment baseline, according to the 2005 European consensus grading system. In one embodiment, the invention may provide the use of an anti-TIM-3 antibody molecule, alone or in combination with ruxolitinib or a pharmaceutically acceptable salt thereof, for treatment of myelofibrosis, particularly for the treatment of bone marrow fibrosis associated with MF, resulting in stabilizing bone marrow fibrosis or improving bone marrow fibrosis from pre-treatment baseline to, for example, week 24 or week 48 of treatment.

The term “constitutional symptoms associated with myelofibrosis”, as used herein, refers to common debilitating chronic myelofibrosis symptoms, such as fever, pruritus (i.e. itching), abdominal pain/discomfort, weight loss, fatigue, inactivity, early satiety, night sweats or bone pain; for example, as described by Mughal et al (Int J Gen Med. 2014 Jan. 29; 7:89-101).

The term “treatment of constitutional symptoms associated with myelofibrosis”, as used herein, refers to “improvement of constitutional symptoms associated with myelofibrosis”, for example, in comparison to the pre-treatment situation or in comparison to best available therapy or to placebo control, for example, a reduction in total symptom score as measured by the modified myelofibrosis symptom assessment form version 2.0 diary (modified MFSAF v2.0) (Cancer 2011; 117:4869-77; N Engl J Med 2012; 366:799-807, the entire contents of which are incorporated herein by reference). In one embodiment, the invention may provide the use of an anti-TIM-3 antibody molecule, alone or in combination with ruxolitinib or a pharmaceutically acceptable salt thereof, for treatment of myelofibrosis, particularly for the treatment of constitutional symptoms associated with myelofibrosis, resulting in improvement of constitutional symptoms associated with myelofibrosis from pre-treatment baseline to, for example, week 24 or week 48 of treatment.

In another embodiment of any use of the invention, one or more of the constitutional symptoms associated with MF are alleviated (e.g. by eliminating or by reducing intensity, duration or frequency). In one embodiment, the reduction of constitutional symptoms is at least ≥20%, at least ≥30%, at least ≥40%, or at least ≥50% as assessed by the modified MFSAF v2.0 from pre-treatment baseline to, for example, week 24 or week 48.

In one embodiment of any use of the invention, the anti-TIM-3 antibody molecule, is administered subsequently or prior to splenectomy or radiotherapy, such as splenic irradiation.

### Combination Therapy

In one aspect the present invention provides an anti-TIM-3 antibody molecule, for use in the treatment of MF, wherein the anti-TIM-3 antibody molecule is administered in combination with at least one further active agent.

In one embodiment the at least one agent is an inhibitor of a non-receptor tyrosine kinases, the Janus kinases (JAK). A considerable number of cytokine and growth factor receptors utilize non-receptor tyrosine kinases, the Janus kinases (JAK), to transmit extracellular ligand binding into an intracellular response. For example, erythropoietin, thrombopoietin and granulocyte monocyte colony stimulating factor are all known to signal through receptors that utilize JAK2. JAK activate a number of downstream pathways implicated in proliferation and survival, including the STATs (signal transducers and activators of transcription), a family of important latent transcription factors.

Accordingly, the present invention relates to the combination use of anti-TIM-3 antibody molecule, with at least one JAK inhibitor, suitably ruxolitinib or a pharmaceutically acceptable salt thereof.

In one embodiment the at least one further active agent is a JAK1/JAK2 inhibitor, suitably ruxolitinib or a pharmaceutically acceptable salt thereof or momelotinib or a pharmaceutically acceptable salt thereof, more suitably ruxolitinib or a pharmaceutically acceptable salt, more suitably ruxolitinib phosphate.

Ruxolitinib represents a novel, potent, and selective inhibitor of JAK1 and JAK2. Ruxolitinib potently inhibits JAK1 and JAK2 [half maximal inhibitory concentration (IC50) 0.4 to 1.7 nM], yet it does not significantly inhibit (<30% inhibition) a broad panel of 26 kinases when tested at 200 nM (approximately 100× the average IC50 value for JAK enzyme inhibition) and does not inhibit JAK3 at clinically relevant concentrations.

In one embodiment the at least one further active agent is a JAK2/FLT3 inhibitor, suitably pacritinib or a pharmaceutically acceptable salt thereof or fedratinib or a pharmaceutically acceptable salt thereof.

In one embodiment the at least one further active agent is a JAK2^{V617F} inhibitor, suitably gandotinib or a pharmaceutically acceptable salt thereof.

In one embodiment the at least one further active agent is a JAK2 inhibitor, suitably BMS-911543 or a pharmaceutically acceptable salt thereof.

In one embodiment the at least one further active agent is a JAK1 inhibitor, suitably itacitinib or a pharmaceutically acceptable salt thereof, in particular itacitinib adipate.

In one embodiment the at least one further active agent is a JAK2/Src inhibitor, suitably NS-018 or a pharmaceutically acceptable salt thereof.

In one aspect the present invention provides a pharmaceutical combination, separate, comprising, consisting essentially of or consisting of an anti-TIM-3 antibody molecule or a pharmaceutical acceptable salt thereof, and b) a JAK1/2 inhibitor, suitably ruxolitinib or a pharmaceutically acceptable salt thereof. Suitably the pharmaceutical combination is for use in the treatment of myelofibrosis.

In one aspect the present invention provides an anti-TIM-3 antibody molecule for use in the treatment of myelofibrosis, wherein or a pharmaceutical acceptable salt thereof, is administered in combination with ruxolitinib or a pharmaceutically acceptable salt thereof, and wherein the anti-TIM-3 antibody molecule and ruxolitinib or a pharmaceutically acceptable salt thereof, are administered in jointly therapeutically effective amounts.

In one aspect the present invention provides ruxolitinib or a pharmaceutically acceptable salt thereof, for use in the treatment of myelofibrosis, wherein ruxolitinib or a pharmaceutically acceptable salt thereof, is administered in combination with an anti-TIM-3 antibody molecule, and wherein ruxolitinib or a pharmaceutically acceptable salt thereof, and an anti-TIM-3 antibody molecule, are administered in jointly therapeutically effective amounts.

The term “combination” or “pharmaceutical combination” used herein, refers to a non-fixed combination where an active agent and at least one further active agent may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time.

The term “non-fixed combination” means that the active ingredients, e.g. one active agent and at least one further active agent, are both administered to a patient as separate entities either simultaneously or sequentially with no specific time limits, wherein such administration provides therapeutically effective levels of the two compounds in the body of the patient. In particular, reference to an anti-TIM-3 antibody molecule in combination with ruxolitinib or a pharmaceutically acceptable salt thereof as used herein (e.g. in any of the embodiments or in any of the claims herein), refers to a “non-fixed combination”; and reference to ruxolitinib or a pharmaceutically acceptable salt thereof as used herein (e.g. in any of the embodiments or in any of the claims herein), in combination with at least one further active agent (an anti-TIM-3 antibody molecule being excluded) refers to either a fixed combination in one unit dosage form (e.g., capsule, tablet, caplets or particulates), a non-fixed combination, or a kit-of-parts for the combined administration wherein ruxolitinib or a pharmaceutically acceptable salt thereof and one or more combination partner (e.g. another drug as specified herein, also referred to as further “pharmaceutical active ingredient”, “therapeutic agent” or “co-agent”) may be administered independently at the same time or separately within time intervals.

The term “therapeutically effective amount” refers to an amount of a drug or a therapeutic agent that will elicit the desired biological and/or medical response of a tissue, system or an animal (including man) that is being sought by a researcher or clinician.

### Administration and Treatment Regimen

In one aspect the present invention provides that the anti-TIM-3 antibody molecule is administered or used at a flat or fixed dose.

In one aspect, the disclosure features a method includes administering to the subject an anti-TIM-3 antibody molecule, e.g., an anti-TIM-3 antibody molecule described herein, at a dose of about 10 mg to about 50 mg, about 50 mg to about 100 mg, about 200 mg to about 300 mg, about 500 mg to about 1000 mg, or about 1000 mg to about 1500 mg, once every two or every four weeks.

In certain embodiments, the anti-TIM-3 antibody molecule is administered at a dose of about 10 mg to about 50 mg once every two or once every four weeks. In other embodiments, the anti-TIM-3 antibody molecule is administered at a dose of about 50 mg to about 100 mg once every two or four weeks. In other embodiments, the anti-TIM-3 antibody molecule is administered at a dose of about 200 mg to about 300 mg once every two or every four weeks. In other embodiments, the anti-TIM-3 antibody molecule is administered at a dose of about 500 mg to about 1000 mg once every two or four weeks. In other embodiments, the anti-TIM-3 antibody molecule is administered at a dose of about 1000 mg to about 1500 mg once every two or every four weeks.

In some embodiments, the anti-TIM-3 antibody molecule is administered at a dose of about 5 mg to about 50 mg, e.g., about 8 mg to about 40 mg, about 10 mg to about 30 mg, about 15 mg to about 35 mg, about 15 mg to about 25 mg, about 5 mg to about 25 mg, about 25 mg to about 50 mg, e.g., about 5 mg, about 10 mg, about 15 mg, about 20 mg, about 25 mg, about 30 mg, about 35 mg, or about 40 mg, once every two weeks. In certain embodiments, the anti-TIM-3 antibody molecule is administered at a dose of about 10 mg to about 30 mg, e.g., about 20 mg, once every two weeks.

In some embodiments, the anti-TIM-3 antibody molecule is administered at a dose of about 50 mg to about 100 mg, e.g., about 60 mg to about 100 mg, about 70 mg to about 90 mg, about 75 mg to about 85 mg, about 50 mg to about 60 mg, about 50 mg to about 80 mg, about 80 mg to about 100 mg, about 60 mg to about 100 mg, e.g., about 50 mg, about 60 mg, about 70 mg, about 80 mg, about 90 mg, or about 100 mg, once every two weeks. In certain embodiments, the anti-TIM-3 antibody molecule is administered at a dose of about 60 mg to about 100 mg, e.g., about 80 mg, once every two weeks.

In other embodiments, the anti-TIM-3 antibody molecule is administered at a dose of about 50 mg to about 100 mg, e.g., about 60 mg to about 100 mg, about 70 mg to about 90 mg, about 75 mg to about 85 mg, about 50 mg to about 60 mg, about 50 mg to about 80 mg, about 80 mg to about 100 mg, about 60 mg to about 100 mg, e.g., about 50 mg, about 60 mg, about 70 mg, about 80 mg, about 90 mg, or about 100 mg, once every four weeks. In certain embodiments, the anti-TIM-3 antibody molecule is administered at a dose of about 60 mg to about 100 mg, e.g., about 80 mg, once every four weeks.

In some embodiments, the anti-TIM-3 antibody molecule is administered at a dose of about 200 mg to about 300 mg, e.g., about 200 mg to about 280 mg, about 200 mg to about 250 mg, about 210 mg to about 270 mg, about 220 mg to about 260 mg, about 230 mg to about 250 mg, about 200 mg to about 220 mg, about 200 mg to about 240 mg, about 200 mg to about 260 mg, about 200 mg to about 280 mg, about 280 to about 300 mg, about 260 to about 300 mg, about 240 to about 300 mg, about 220 to about 300 mg, e.g., about 200 mg, about 240 mg, about 260 mg, about 280 mg, or about 300 mg, once every two weeks. In certain embodiments, the anti-TIM-3 antibody molecule is administered at a dose of about 220 mg to about 260 mg, e.g., about 240 mg, once every two weeks.

In some embodiments, the anti-TIM-3 antibody molecule is administered at a dose of about 200 mg to about 300 mg, e.g., about 200 mg to about 280 mg, about 200 mg to about 250 mg, about 210 mg to about 270 mg, about 220 mg to about 260 mg, about 230 mg to about 250 mg, about 200 mg to about 220 mg, about 200 mg to about 240 mg, about 200 mg to about 260 mg, about 200 mg to about 280 mg, about 280 to about 300 mg, about 260 to about 300 mg, about 240 to about 300 mg, about 220 to about 300 mg, e.g., about 200 mg, about 240 mg, about 260 mg, about 280 mg, or about 300 mg, once every four weeks. In certain embodiments, the anti-TIM-3 antibody molecule is administered at a dose of about 220 mg to about 260 mg, e.g., about 240 mg, once every four weeks.

In some embodiments, the anti-TIM-3 antibody molecule is administered at a dose of about 500 mg to about 1000 mg, e.g., about 600 mg to about 1000 mg, about 700 mg to about 900 mg, about 750 mg to about 850 mg, about 500 mg to about 600 mg, about 500 mg to about 800 mg, about 800 mg to about 1000 mg, about 600 mg to about 1000 mg, e.g., about 500 mg, about 600 mg, about 700 mg, about 800 mg, about 900 mg, or about 1000 mg, once every two weeks. In certain embodiments, the anti-TIM-3 antibody molecule is administered at a dose of about 600 mg to about 1000 mg, e.g., about 800 mg, once every two weeks.

In some embodiments, the anti-TIM-3 antibody molecule is administered at a dose of about 500 mg to about 1000 mg, e.g., about 600 mg to about 1000 mg, about 700 mg to about 900 mg, about 750 mg to about 850 mg, about 500 mg to about 600 mg, about 500 mg to about 800 mg, about 800 mg to about 1000 mg, about 600 mg to about 1000 mg, e.g., about 500 mg, about 600 mg, about 700 mg, about 800 mg, about 900 mg, or about 1000 mg, once every four weeks. In certain embodiments, the anti-TIM-3 antibody molecule is administered at a dose of about 600 mg to about 1000 mg, e.g., about 800 mg, once every four weeks.

In some embodiments, the anti-TIM-3 antibody molecule is administered at a dose of about 1000 mg to about 1500 mg, e.g., about 1000 mg to about 1400 mg, about 1100 mg to about 1300 mg, about 1000 mg to about 1200 mg, about 1000 mg to about 1400 mg, about 1300 mg to about 1500 mg, about 1100 mg to about 1500 mg, about 1200 mg to about 1400 mg, about 1000 mg to about 1300 mg, about 1100 mg to about 1400 mg, about 1200 mg to about 1500 mg, e.g., about 1000 mg, about 1100 mg, about 1200 mg, about 1300 mg, about 1400 mg, or about 1500 mg, once every two weeks. In certain embodiments, the anti-TIM-3 antibody molecule is administered at a dose of about 1100 mg to about 1300 mg, e.g., about 1200 mg, once every two weeks.

In some embodiments, the anti-TIM-3 antibody molecule is administered at a dose of about 1000 mg to about 1500 mg, e.g., about 1000 mg to about 1400 mg, about 1100 mg to about 1300 mg, about 1000 mg to about 1200 mg, about 1000 mg to about 1400 mg, about 1300 mg to about 1500 mg, about 1100 mg to about 1500 mg, about 1200 mg to about 1400 mg, about 1000 mg to about 1300 mg, about 1100 mg to about 1400 mg, about 1200 mg to about 1500 mg, e.g., about 1000 mg, about 1100 mg, about 1200 mg, about 1300 mg, about 1400 mg, or about 1500 mg, once every four weeks. In certain embodiments, the anti-TIM-3 antibody molecule is administered at a dose of about 1100 mg to about 1300 mg, e.g., about 1200 mg, once every four weeks.

Suitably the anti-TIM-3 antibody molecule, is provided to the subject intravenously.

In one embodiment the present invention provides the anti-TIM-3 antibody molecule, for use in the treatment of myelofibrosis, wherein said anti-TIM-3 antibody molecule, is administered in combination with ruxolitinib, or a pharmaceutically acceptable salt thereof. Suitably ruxolitinib is administered in an amount of from 5 mg twice daily to 25 mg twice daily, such as 5 mg twice daily, 10 mg twice daily, 15 mg twice daily, 20 mg twice daily or 25 mg twice daily, depending on the patient's blood count according to the prescribing information for Jakavi®/Jakafi® and the judgment of the treating physician.

All publications and patent applications mentioned in the specification are indicative of the level of skill of those skilled in the art to which the presently disclosed inventive concepts pertain. All publications and patent applications are herein incorporated by reference to the same extent as if each individual publication or patent application was specifically and individually indicated to be incorporated by reference.

### LIST OF ABBREVIATIONS

AE adverse event
AML Acute myeloid leukemia
ANC Absolute neutrophil count
ASCT allogeneic hematopoietic stem cell transplantation
AUC Area under curve
BID twice a day
BM bone marrow
C1D1 Cycle 1 Day 1 (and sequentially for other cycles and days, eg C1D2, C2D1 etc.)
CT computed tomography
CTCAE Common Terminology Criteria for Adverse Events
CYP cytochrome P-450
DDI Drug-drug interaction
DLT dose-limiting toxicity
ECG Electrocardiogram
EORTC European Organization for Research and Treatment of Cancer
ET essential thrombocythemia
Hb hemoglobin
IV Intravenous
IWG-MRT International Working Group-Myeloproliferative Neoplasms Research and Treatment
JAK Janus kinase
LCM left costal margin
MF myelofibrosis
MPN myeloproliferative neoplasm
MRI magnetic resonance imaging
PD pharmacodynamic(s)
PFS progression free survival
PK pharmacokinetic(s)
PLT platelets
PMF primary myelofibrosis
PRBC packed red blood cells
PV polycythemia vera
QD once a day
QLQ-C30 Quality of Life Questionnaire-Core 30
QoL quality of life
RBC red blood cell(s)
RP2D recommended phase 2 dose
RR Response rate
SAF symptom assessment form
STAT signal transducer and activator of transcription
TIM-3 T Cell Immunoglobulin Mucin 3
TLS Tumor lysis syndrome
TSS total symptom score
WHO World Health Organization

The Examples below are set forth to aid in the understanding of the inventions but are not intended to, and should not be construed to, limit its scope in any way.

### EXPERIMENTAL

### A Randomized, Open-Label, Phase I/II Open Platform Study Evaluating Safety and Efficacy of Novel Ruxolitinib Combination in Myelofibrosis Patients

Rationale for Dose/Regimen and Duration of Treatment for an Anti-TIM-3 Antibody Molecule in Combination with Ruxolitinib

This is the first trial that will evaluate the combination of anti-TIM-3 antibody molecule with ruxolitinib.

In this study, the selection of the MBG453 dose and regimen is based on the currently available clinical safety, efficacy, PK and PK/PD modeling information from the CMBG453X2101 in advanced solid tumors and CPDR001X2105 in AML and MDS trials for MBG453. For solid tumor subjects, both 800 mg IV Q4W and 400 mg IV every 2 weeks (Q2W) are predicted to give sustained target (TIM-3) occupancy of 90% in tumor in >90% of subjects. No significant safety signal has been detected at any dose of MBG453 up to 1200 mg IV Q2W or Q4W in the CMBG453X2101 study. MBG453 single agent is also being evaluated in AML/MDS subjects in the CPDR001X2105 study with Q4W and Q2W regimens. The recommended dose in AML/MDS has not yet been determined, however it is not expected to be different from solid tumors, based on preliminary PK and safety data. MBG453 at the dose levels of 400 mg IV Q2W and 800 mg IV Q4W has been well tolerated in AMUMDS. In this study, 800 mg IV Q4W has been selected as the MBG453 dose regimen for combination with ruxolitinib.

Ruxolitinib is associated with partial transient hematoxicity. As MBG453 has not been found to have a negative impact on hematopoiesis in its phase I/II development, there are no overlapping hematoxicities expected for the ruxolitinib+MBG4563 combination treatment.

As a monoclonal antibody, MBG453 is eliminated through protein catabolism and targetmediated disposition. However, immunomodulators such as MBG453 may induce systemic cytokines that alter CYP-mediated metabolism and affect the clearance of small molecules (Girish et al 2011, Lee et al 2010, Huang et al 2010). Therefore, the risk of PK DDI between MBG453 and ruxolitinib cannot be totally excluded, although it is anticipated to be low.

The purpose of this study is to investigate the safety, pharmacokinetics and preliminary efficacy of combination treatment of ruxolitinib with MBG-453 in MF subjects. The study consists of three parts:

Part 1: Dose escalation and safety run-in (recommended Phase II dose confirmation)

Part 2: Selection

Part 3: Expansion

Purpose of Rationale:

Myelofibrosis (MF) is defined by progressive bone marrow (BM) fibrosis and a consecutive reduction of blood cells. The disruption of the medullary erythropoietic niche is the primary mechanism governing the bone marrow failure and anemia, which typify MF. Nearly 40% of MF patients have hemoglobin (Hb) levels <10 g/dL at diagnosis. Furthermore, anemia is the disease feature most consistently associated with poor prognosis in MF.

Ruxolitinib demonstrates improvements in splenomegaly and constitutional symptoms, however, does not improve anemia.

The purpose of this study is to investigate the safety, pharmacokinetics (PK) and preliminary efficacy of combinations treatment of ruxolitinib with novel anti-TIM-3 antibody MGB-453 in MF subjects. This combination therapy may deliver transformational clinical benefits such as improvement of progression free survival (PFS) as a consequence of superior disease control or reduction of the malignant clone, associated with an improvement of cytopenia and in particular anemia, as well as improvement in quality of life (QoL) as captured by relevant patient reported outcomes measurements (PROs).

Key Inclusion Criteria:

Subjects have diagnosis of primary myelofibrosis (PMF) according to the 2016 World Health Organization (WHO) criteria, or diagnosis of postessential thrombocythemia (ET) (PET-MF) or post-polycythemia vera (PV) myelofibrosis (PPV-MF) according to the International Working Group for Myelofibrosis Research and Treatment (IWG-MRT) 2007 criteria;

Palpable spleen of at least 5 cm from the left costal margin (LCM) to the point of greatest splenic protrusion or enlarged spleen volume of at least 450 cm3 per MRI or CT scan at baseline (a MRI/CT scan up to 8 weeks prior to first dose of study treatment can be accepted).

Have been treated with ruxolitinib for at least 24 weeks prior to first dose of study treatment.

Are stable (no dose adjustments) on the prescribed ruxolitinib dose (between 5 and 25 mg twice a day (BID)) for ≥8 weeks prior to first dose of study treatment.

Hemoglobin <10 g/dL

Part 1: Platelet counts ≥75 000/μL

Part 2 and Part 3: Platelet counts ≥50 000/μL.

Key Exclusion Criteria

Not able to understand and to comply with study instructions and requirements.

Received any investigational agent for the treatment of MF (except ruxolitinib) within 30 days of first dose of study treatment or within 5 halflives of the study treatment, whichever is greater.

Peripheral blood blasts count of >10%.

Received a monoclonal antibody (Ab) or immunoglobulin-based agent within 1 year of screening, or has documented severe hypersensitivity reactions/immunogenicity (IG) to a prior biologic.

Splenic irradiation within 6 months prior to the first dose of study drug.

Received blood platelet transfusion within 28 days prior to first dose of study treatment.

Subjects with known TP53 mutation or deletion of TP53.

Primary Objectives:

To evaluate the preliminary efficacy of each novel ruxolitinib combination treatment arm (Parts 2 & 3)

To characterize the safety, tolerability, and recommended phase 2 dose (RP2D) of combination partner used with ruxolitinib (Part 1)

Primary Endponts:

Response rate (RR) for the composite endpoint (anemia improvement of ≥1.5 g/dL and no spleen volume progression and no symptom worsening) at the end of Cycle 6.

Incidence and severity of dose limiting toxicity (DLTs) within the first 2 treatment cycles in Part 1 of the study

Secondary Objectives:

To assess the proportion of subjects in each treatment arm who achieved an Hb improvement of ≥2.0 g/dL or ≥1.5 g/dL (Parts 2 & 3).

To evaluate changes in symptoms of myelofibrosis in each treatment arm using MFSAF v4.0 and EORTC QLQ-C30 patient reported outcomes (PROs) (Parts 2 & 3).

To characterize the pharmacokinetic profile of ruxolitinib administered in combination with anti-TIM-3 antibody MGB-453 (Parts 1, 2 & 3).

To assess emergence of anti-MBG453 antibodies following one or more IV infusions (Parts 1, 2 & 3).

To evaluate the changes in spleen size in each treatment arm (Parts 2 & 3).

To evaluate the effect of ruxolitinib combination treatment in delaying progression of MF and estimate time to progression free survival (PFS) event (Parts 2 & 3).

To evaluate the effect on bone marrow fibrosis in each treatment arm (Parts 2 & 3).

To evaluate long-term safety and tolerability of ruxolitinib combination treatments (Parts 1, 2 & 3).

Secondary Endppoints:

Change in MFSAF v4.0 and EORTC QLQ-C30 from Baseline.

PK parameters (e.g., AUC, Cmax, Tmax) and concentration vs. time profiles of each investigational drug within combination regimens.

Presence and/or concentration of anti-MBG453 antibodies.

Change in spleen length (by palpation) from baseline.

Change in spleen volume (by MRI/CT) from baseline.

Estimate of progression free survival (PFS) where events are defined as follows:

Progressive splenomegaly as assessed by increasing spleen volume (by MRI/CT) of ≥25% from baseline. The progression date will be the date of MRI/CT assessment confirming spleen volume increase of ≥25% from baseline;

Accelerated phase defined by a circulating peripheral blood blast content of >10% but <20% confirmed after 2 weeks. The progression date will be the date of first increase in peripheral blood blast content of >10%;

Deteriorating cytopenia (dCP) independent from treatment defined for all patients by platelet count <35×10\hat{ }9/L or neutrophil count <0.75×10\hat{ }9/L that lasts for at least 4 weeks. The progression date will be the date of first decrease of platelets <35×10\hat{ }9/L or neutrophils <0.75×10\hat{ }9/L confirmed after 4 weeks;

Leukemic transformation defined by a peripheral blood blast content of ≥20% associated with an absolute blast count of ≥1×10\hat{ }9/L that lasts for at least 2 weeks or a bone marrow blast count of ≥20%. The progression date will be the date of first increase in peripheral blood blast content of ≥20% associated with an absolute blast count of ≥1×10\hat{ }9/L OR the date of the bone marrow blast count of ≥20%;

Death from any cause.

Proportion of subjects achieving improvement in bone marrow fibrosis of ≥1 grade from baseline Frequency, duration and severity of adverse events, abnormalities in vital signs and laboratory test values, including ECG data

1. An anti-TIM-3 antibody molecule for use in the treatment of myelofibrosis (MF) in a patient.

2. The anti-TIM-3 antibody molecule for use according to claim 1, wherein myelofibrosis comprises primary myelofibrosis (PMF), post-essential thrombocythemia myelofibrosis (PET-MF) and post-polycythemia vera myelofibrosis (PPV-MF).

3. The anti-TIM-3 antibody molecule for use use according to claim 1 wherein the anti-TIM-3 antibody molecule comprises:
(a) a heavy chain variable region (VH) comprising a VHCDR1 amino acid sequence chosen from SEQ ID NO: 9; a VHCDR2 amino acid sequence of SEQ ID NO: 10; and a VHCDR3 amino acid sequence of SEQ ID NO: 5; and a light chain variable region (VL) comprising a VLCDR1 amino acid sequence of SEQ ID NO: 12, a VLCDR2 amino acid sequence of SEQ ID NO: 13, and a VLCDR3 amino acid sequence of SEQ ID NO: 14;
(b) a VH comprising a VHCDR1 amino acid sequence chosen from SEQ ID NO: 3; a VHCDR2 amino acid sequence of SEQ ID NO: 4; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8;
(c) a VH comprising a VHCDR1 amino acid sequence chosen from SEQ ID NO: 9; a VHCDR2 amino acid sequence of SEQ ID NO: 25; and a VHCDR3 amino acid sequence of SEQ ID NO: 5; and a VL comprising a VLCDR1 amino acid sequence of SEQ ID NO: 12, a VLCDR2 amino acid sequence of SEQ ID NO: 13, and a VLCDR3 amino acid sequence of SEQ ID NO: 14;
(d) a VH comprising a VHCDR1 amino acid sequence chosen from SEQ ID NO: 3; a VHCDR2 amino acid sequence of SEQ ID NO: 24; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8;
(e) a VH comprising a VHCDR1 amino acid sequence chosen from SEQ ID NO: 9; a VHCDR2 amino acid sequence of SEQ ID NO: 31; and a VHCDR3 amino acid sequence of SEQ ID NO:
5; and a VL comprising a VLCDR1 amino acid sequence of SEQ ID NO: 12, a VLCDR2 amino acid sequence of SEQ ID NO: 13, and a VLCDR3 amino acid sequence of SEQ ID NO: 14; or
(f) a VH comprising a VHCDR1 amino acid sequence chosen from SEQ ID NO: 3; a VHCDR2 amino acid sequence of SEQ ID NO: 30; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8.

4. The anti-TIM-3 antibody molecule for use use according to claim 1, wherein myelofibrosis is primary myelofibrosis (PMF).

5. The anti-TIM-3 antibody molecule for use according to claim 1, wherein median survival time increases by at least 3 months.

6. The anti-TIM-3 antibody molecule for use according to claim 1, wherein said patient completely responds to the treatment.

7. The anti-TIM-3 antibody molecule for use according to claim 1, wherein said MF is newly diagnosed MF.

8. The anti-TIM-3 antibody molecule for use according to claim 1, wherein said anti-TIM-3 antibody molecule is administered in combination with at least one further active agent.

9. The anti-TIM-3 antibody molecule for use according to claim 1, wherein the at least one further active agent is a JAK1/JAK2 inhibitor, a JAK2/FLT3 inhibitor, a JAK2^{V617F} inhibitor, a JAK2 inhibitor, JAK1 inhibitor or a JAK2/Src inhibitor, such as ruxolitinib, or a pharmaceutically acceptable salt thereof.

10. The anti-TIM-3 antibody molecule for use according to claim 9, wherein ruxolitinib or a pharmaceutically acceptable salt thereof, is administered in an amount of from 5 mg twice daily to 25 mg twice daily, such as 5 mg twice daily, 10 mg twice daily, 15 mg twice daily, 20 mg twice daily or 25 mg twice daily.

11. The anti-TIM-3 antibody molecule for use according to claim 1, wherein the anti-TIM-3 antibody is administered in an amount of about 10 mg to about 50 mg, about 50 mg to about 100 mg, about 200 mg to about 300 mg, about 500 mg to about 1000 mg, or about 1000 mg to about 1500 mg, once every two or every four weeks.

12. The anti-TIM-3 antibody molecule for use according to claim 1, wherein the anti-TIM-3 antibody is administered in an amount of about 400 mg every two weeks, 600 mg every three weeks, or about 800 mg every four weeks.

13. The anti-TIM-3 antibody molecule for use according to claim 1, wherein the anti-TIM-3 antibody is administered in an amount of 800 mg every four weeks.

14. The anti-TIM-3 antibody molecule for use according to claim 1, wherein the anti-TIM-3 antibody molecule comprises:
(a) a heavy chain variable domain comprising the amino acid sequence of SEQ ID NO: 26 and a light chain variable domain comprising the amino acid sequence of SEQ ID NO: 20;
(b) a heavy chain variable domain comprising the amino acid sequence of SEQ ID NO: 32 and a light chain variable domain comprising the amino acid sequence of SEQ ID NO: 20; or
(c) a heavy chain variable domain comprising the amino acid sequence of SEQ ID NO: 52 and a light chain variable domain comprising the amino acid sequence of SEQ ID NO: 64;

15. The anti-TIM-3 antibody molecule for use according to claim 1, wherein the anti-TIM-3 antibody molecule comprises:
a) a heavy chain comprising the amino acid sequence of SEQ ID NO: 28 and a light chain variable domain comprising the amino acid sequence of SEQ ID NO: 22;
b) a heavy chain comprising the amino acid sequence of SEQ ID NO: 34 and a light chain variable domain comprising the amino acid sequence of SEQ ID NO: 22; or
c) a heavy chain comprising the amino acid sequence of SEQ ID NO: 54 and a light chain variable domain comprising the amino acid sequence of SEQ ID NO: 66.

16. An anti-TIM-3 antibody molecule for use in the treatment of myelofibrosis (MF) in a patient,
wherein the anti-TIM-3 antibody molecule binds to the same epitope as, or an epitope that overlaps with, the epitope as a monoclonal antibody to human TIM-3, wherein the monoclonal antibody comprises:
(a) a heavy chain variable region (VH) comprising a VHCDR1 amino acid sequence chosen from SEQ ID NO: 9; a VHCDR2 amino acid sequence of SEQ ID NO: 10; and a VHCDR3 amino acid sequence of SEQ ID NO: 5; and a light chain variable region (VL) comprising a VLCDR1 amino acid sequence of SEQ ID NO: 12, a VLCDR2 amino acid sequence of SEQ ID NO: 13, and a VLCDR3 amino acid sequence of SEQ ID NO: 14;
(b) a VH comprising a VHCDR1 amino acid sequence chosen from SEQ ID NO: 3; a VHCDR2 amino acid sequence of SEQ ID NO: 4; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8;
(c) a VH comprising a VHCDR1 amino acid sequence chosen from SEQ ID NO: 9; a VHCDR2 amino acid sequence of SEQ ID NO: 25; and a VHCDR3 amino acid sequence of SEQ ID NO: 5; and a VL comprising a VLCDR1 amino acid sequence of SEQ ID NO: 12, a VLCDR2 amino acid sequence of SEQ ID NO: 13, and a VLCDR3 amino acid sequence of SEQ ID NO: 14;
(d) a VH comprising a VHCDR1 amino acid sequence chosen from SEQ ID NO: 3; a VHCDR2 amino acid sequence of SEQ ID NO: 24; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8;
(e) a VH comprising a VHCDR1 amino acid sequence chosen from SEQ ID NO: 9; a VHCDR2 amino acid sequence of SEQ ID NO: 31; and a VHCDR3 amino acid sequence of SEQ ID NO: 5; and a VL comprising a VLCDR1 amino acid sequence of SEQ ID NO: 12, a VLCDR2 amino acid sequence of SEQ ID NO: 13, and a VLCDR3 amino acid sequence of SEQ ID NO: 14; or
(f) a VH comprising a VHCDR1 amino acid sequence chosen from SEQ ID NO: 3; a VHCDR2 amino acid sequence of SEQ ID NO: 30; and a VHCDR3 amino acid sequence of SEQ ID NO: 5; and a VL comprising a VLCDR1 amino acid sequence of SEQ ID NO: 6, a VLCDR2 amino acid sequence of SEQ ID NO: 7, and a VLCDR3 amino acid sequence of SEQ ID NO: 8, wherein:
(1) the antibody molecule binds to one, two, three, or all of: the two residues adjacent to the N-terminus of the A strand (Val24 and Glu25 in human TIM-3), the BC loop, the CC′ loop, or the G strand of human TIM-3; and
(2) the antibody molecule has one, two, three, four, five, six, seven or all of the following properties:
(i) reduces PtdSer-dependent membrane penetration of TIM-3;
(ii) reduces binding of TIM-3 to one, two, or all of PtdSer, HMGB1, or CEACAM-1;
(iii) does not inhibit binding of TIM-3 to Galectin-9;
(iv) competes with CEACAM-1 for binding to one, two, or all of Cys58, Asn119 and Lys122 of TIM-3;
(v) reduces the formation of a hydrogen bond between Lys122 of TIM-3 and Asn42 of CEACAM-1;
(vi) competes with PtdSer for binding to the FG loop and the CC′ loop of TIM-3;
(vii) competes with HMGB1 for binding to Glu62 of TIM-3; or
(viii) does not compete with Galectin-9 for binding to TIM-3.
